(19) United States
(12) Patent Application Publication
(10) Pub. No.: US 2025/0154287 A1
(43) Pub. Date: May 15, 2025

(54) ANTI-MIGIS-alpha ANTIBODIES AND METHODS OF USE THEREOF

(71) Applicants: Pfizer Inc., New York, NY (US); Benaroya Research Institute at Virginia Mason, Seattle, WA (US)

(72) Inventors: Edward Michael FRANKLIN, Dublin (IE); Brian Joseph FENNELL, Dublin (IE); Edwina Catherine STACK, Dublin (IE); Lidia MOSYAK, Newton, MA (US); James Reasoner APGAR, Newton, MA (US); Jeffrey Raymond CHABOT, Medford, MA (US); Bernard KHOR, Seattle, WA (US); Thomas Jay MIKITA, Sausalito, CA (US); Javier Fernando CHAPARRO-RIGGERS, San Diego, CA (US)

(21) Appl. No.: 18/942,166

(22) Filed: Nov. 8, 2024

Related U.S. Application Data
(60) Provisional application No. 63/712,818, filed on Oct. 28, 2024.
(60) Provisional application No. 63/597,777, filed on Nov. 10, 2023.

(51) Int. Cl. C07K 16/42; A61P 37/06; C07K 16/28

(52) U.S. Cl. CPC C07K 16/4283; A61P 37/06; C07K 16/2809; C07K 2317/31; C07K 2317/565; C07K 2317/567; C07K 2317/732; C07K 2317/92

(57) ABSTRACT

Provided herein are antibodies (including antigen-binding fragments thereof) that specifically bind to MIGIS-α, including for example without limitation, bispecific MIGIS-α/CD3 antibodies, other related antibodies, related nucleic acids, uses, and associated methods thereof. The disclosure also provides processes for making, preparing, and producing antibodies disclosed herein, including antibodies that bind to one or both of MIGIS-α and CD3.

### RELATED APPLICATIONS

This application claims priority to and the benefit of U.S. Provisional Patent Applications 63/597,777, filed Nov. 10, 2023 and 63/712,818, filed Oct. 28, 2024. Each of the foregoing applications is incorporated herein by reference in its entirety.

### SEQUENCE LISTING

The instant application contains a Sequence Listing which has been submitted electronically in XML format and is hereby incorporated by reference in its entirety. Said XML copy, created on Nov. 8, 2024, is named PCFC-0080-101-SL.xml and is 94,091 bytes in size.

### FIELD

The present invention relates to antibodies that specifically bind to MIGIS-α. The present invention further relates to antibodies that bind to MIGIS-α, and CD3. The present invention also pertains to related molecules, e.g. nucleic acids which encode such antibodies, compositions, and related methods, e.g., methods for producing and purifying such antibodies, and their use in diagnostics and therapeutics.

### BACKGROUND

IgA nephropathy (IgAN), also called Berger's disease, is an autoimmune disease impairing kidney function, with an average worldwide incidence of 2.5 per 100,000 per year (Zaidi, O., et al., BMC Nephrol 25, 136, 2024). As the most common form of primary glomerulonephritis worldwide there is a significant unmet need; approximately 1 in 4 patients progress to end stage renal disease or experience a 50% decline in estimated glomerular filtration rate (eGFR) within 10 years (Coppo, R., et al., Kidney Int, 86(4): p. 828-36, 2014.

The pathogenesis of IgAN is widely accepted to follow a multi-hit process Novak, J., et al., Semin Immunopathol 34, 365-382, 2012). IgAN is driven by the pathogenic production of aberrantly glycosylated IgA (deficient in O-linked galactose), via an interplay of polygenic and environmental factors that are not well understood, leading to the aberrant production of serum IgA1. The other subclass of IgA, IgA2, is not O-glycosylated and is not involved in IgAN. The galactose-deficient IgA1 (Gd-IgA1) molecules in circulation are recognized as autoantigens that are subsequently targeted by IgG and IgA autoantibodies, leading to immune complex formation. IgA-containing immune complexes bind to and activate proliferation of mesangial cells and induce complement activation, inflammation and fibrosis. Diagnosis of IgAN necessitates a kidney biopsy and detection of these glomerular IgA-positive immune complex deposits.

Current treatments for IgAN are limited to broad based symptom management, including angiotensin-converting enzyme inhibitors and angiotensin receptor blockers to control high blood pressure and swelling (edema); corticosteroids and other immunosuppressive agents; cholesterol lowering agents; and fish oil and dietary management of fluids, salts, and protein intake. In the majority of cases, the prognosis is for the condition to progressively worsen, ultimately requiring treatment for chronic kidney disease and possibly dialysis and transplant.

More than 10 therapies are in clinical development for the treatment of IgAN (Noor, S. M., et al., Front Nephrol, 3: p. 1175088, 2023). However, there is a lack of IgA-specific therapies. Two therapies that lack IgA-specific mechanisms, budesonide and sparsentan, have recently been granted accelerated approval based on 30-50% reductions in proteinuria (a surrogate endpoint for IgAN trials). Their full approval is dependent on long term eGFR improvements, which sparsentan has apparently failed to meet. There are six therapies in phase 2 or 3 that inhibit the alternative or mannose-lectin complement pathway and may carry safety risks with long-term immunosuppression.

Anti-ARPIL therapies (zigakibart, sibeprenlimab) as well as a dual APRIL/BAFF inhibitor (atacicept) are among the most promising programs under clinical development, as these potentially target production of IgA. Atacicept has shown modest proteinuria reductions to date. Of the two anti-APRILs, zigakibart (BION-1301), a monoclonal antibody in phase 2/3 antagonizing the signaling of the plasma cell survival factor APRIL, lowers circulating IgA, IgM and IgG, and is showing the strongest proteinuria reductions to date (albeit the patient population studied had less severe disease). However, a drawback of anti-APRIL antibodies is the suppression of IgM and IgG levels in addition to IgA.

While anti-MIGIS-α antibody clones have been described: U.S. Pat. No. 9,688,776, (Yamasaki, K., et al., Monoclon Antib Immunodiagn Immunother, 41(3): p. 125-132, 2022, and Alfur Fu-Hsin Hung, et al., Molecular Immunology, Vol: 48, Issue: 15, Page: 1975-1982, 2011), they have failed to demonstrate cell binding and killing of primary cells with endogenous mIgA expression.

### SUMMARY OF THE INVENTION

The present invention related to antibodies that specifically bind membrane immunoglobulin isotype-specific alpha (MIGIS-α), and compositions, methods and uses thereof, including use of anti-MIGIS-α antibodies of the disclosure to treat IgA nephropathy, including treatment and prevention of inflammation of the glomeruli associated with IgA nephropathy. The present invention also pertains to related molecules, e.g. nucleic acids which encode such antibodies or bispecific antibodies, compositions, and related methods, e.g., methods for producing and purifying such antibodies and bispecific antibodies, and their use in diagnostics and therapeutics.

Provided herein are antibodies (including antigen-binding fragments thereof) that specifically bind to MIGIS-α, including for example without limitation, bispecific MIGIS-α/CD3 antibodies, other related antibodies, related nucleic acids, uses, and associated methods thereof. The disclosure also provides processes for making, preparing, and producing antibodies disclosed herein, including antibodies that bind to one or both of MIGIS-α and CD3. Antibodies of the disclosure are useful in one or more of diagnosis, prophylaxis, or treatment of disorders or conditions mediated by, or associated with one or more of MIGIS-α and MIGIS-α/CD3 activity, including, but not limited to in the manufacture of a medicament for treating a disease, disorder or condition mediated by MIGIS-α. In some embodiments, the disease, disorder or condition is at least one selected from the group consisting of IgA nephropathy (IgAN), Berger's disease, primary immunoglobulin A (IgA) nephropathy at risk of rapid disease progression, glomerular proteinuria, hematuria, inflammation of the glomeruli glomerulonephritis, chronic glomerulonephritis, acute glomerulonephritis, Celiac Disease, and Henoch-Schonlein purpura, multiple myeloma (IgA subtype), IgA plasma cell neoplasms, Barrett's Esophagus, rheumatoid arthritis, COPD, Kawasaki Disease, IgA vasculitis, and idiopathic pulmonary fibrosis.

The disclosure further encompasses expression of antibodies, and preparation and manufacture of compositions comprising antibodies of the disclosure, such as medicaments for the use of the antibodies.

Polynucleotides encoding antibodies that specifically bind to MIGIS-α, as well as bispecific MIGIS-α/CD3 antibodies, other related antibodies, are also provided. Polynucleotides encoding antibody heavy chains or light chains, or both are also provided. Host cells that express the antibodies are provided. Methods of treatment using the antibodies are provided. Such methods include, but are not limited to, one or more of methods of treating or methods of preventing diseases associated with or mediated by either or both of MIGIS-α, and MIGIS-α and CD3 expression, or one or more diseases selected from the group consisting of IgA nephropathy (IgAN), Berger's disease, primary immunoglobulin A (IgA) nephropathy at risk of rapid disease progression, glomerular proteinuria, hematuria, inflammation of the glomeruli, glomerulonephritis, chronic glomerulonephritis, acute glomerulonephritis, Henoch-Schonlein purpura, multiple myeloma (IgA subtype), IgA plasma cell neoplasms, Barrett's Esophagus, rheumatoid arthritis, COPD, Kawasaki Disease, IgA vasculitis, and idiopathic pulmonary fibrosis.

The present invention may be understood more readily by reference to the following detailed description of the embodiments of the invention and the Examples included herein. It is to be understood that this invention is not limited to specific methods of making that may of course vary. It is to be also understood that the terminology used herein is for the purpose of describing specific embodiments only and is not intended to be limiting. Those skilled in the art will recognize or be able to ascertain using no more than routine experimentation, many equivalents to the specific embodiments of the invention described herein. Such equivalents are intended to be encompassed by the following embodiments (E).

E1. An isolated antibody or antigen-binding fragment thereof, that binds to human MIGIS-α through a MIGIS-α binding domain, comprising a MIGIS-α variable heavy region (MIGIS-α VH) and a MIGIS-α variable light region (MIGIS-α VL).

E2. The antibody of any one of E1-E3 comprising a CDRL1 selected from the group consisting of SEQ ID NO: 8, SEQ ID NO: 9, SEQ ID NO: 27, SEQ ID NO: 28, SEQ ID NO: 29 and SEQ ID NO: 30; a CDRL2 selected from the group consisting of SEQ ID NO: 10, and SEQ ID NO: 31; a CDRL3 selected from the group consisting of SEQ ID NO: 11, SEQ ID NO: 12, SEQ ID NO: 13, and SEQ ID NO: 32; a CDRH1 selected from the group consisting of SEQ ID NO: 1, SEQ ID NO: 2, SEQ ID NO: 3, SEQ ID NO: 22, and SEQ ID NO: 23; a CDRH2 selected from the group consisting of SEQ ID NO: 4, SEQ ID NO: 5, SEQ ID NO: 24, and SEQ ID NO: 25; a CDRH3 selected from the group consisting of SEQ ID NO: 7, SEQ ID NO: 6, and SEQ ID NO: 26.

E3. The antibody of any one of E1-E2 comprising a CDRL1 selected from the group consisting of SEQ ID NO: 8, and SEQ ID NO: 9; a CDRL2 of SEQ ID NO: 10; a CDRL3 selected from the group consisting of SEQ ID NO: 11, SEQ ID NO: 12, and SEQ ID NO: 13; a CDRH1 selected from the group consisting of SEQ ID NO: 1, SEQ ID NO: 2, and SEQ ID NO: 3; a CDRH2 selected from the group consisting of SEQ ID NO: 4, and SEQ ID NO: 5; a CDRH3 selected from the group consisting of SEQ ID NO: 7, and SEQ ID NO: 6.

E4. The antibody of E1-E3, comprising CDRs selected from the one of the groups in the following list:
(i) a CDR-H sequence comprising the amino acid sequence of SEQ ID NO: 8; a CDR-H2 sequence comprising the amino acid sequence of SEQ ID NO: 10; a CDR-H3 sequence comprising the amino acid sequence of SEQ ID NO: 11; a CDR-L1 sequence comprising the amino acid sequence of SEQ ID NO: 1; a CDR-L2 sequence comprising the amino acid sequence of SEQ ID NO: 4; and a CDR-L3 sequence comprising the amino acid sequence of SEQ ID NO: 7;
(ii) a CDR-H1 sequence comprising the amino acid sequence of SEQ ID NO: 9; a CDR-H2 sequence comprising the amino acid sequence of SEQ ID NO: 10; a CDR-H3 sequence comprising the amino acid sequence of SEQ ID NO: 12; a CDR-L1 sequence comprising the amino acid sequence of SEQ ID NO: 1; a CDR-L2 sequence comprising the amino acid sequence of SEQ ID NO: 4; and a CDR-L3 sequence comprising the amino acid sequence of SEQ ID NO: 7;
(iii) a CDR-H1 sequence comprising the amino acid sequence of SEQ ID NO: 8; a CDR-H2 sequence comprising the amino acid sequence of SEQ ID NO: 10; a CDR-H3 sequence comprising the amino acid sequence of SEQ ID NO: 11; a CDR-L1 sequence comprising the amino acid sequence of SEQ ID NO: 2; a CDR-L2 sequence comprising the amino acid sequence of SEQ ID NO: 4; and a CDR-L3 sequence comprising the amino acid sequence of SEQ ID NO: 7;
(iv) a CDR-H1 sequence comprising the amino acid sequence of SEQ ID NO: 8; a CDR-H2 sequence comprising the amino acid sequence of SEQ ID NO: 10; a CDR-H3 sequence comprising the amino acid sequence of SEQ ID NO: 13; a CDR-L1 sequence comprising the amino acid sequence of SEQ ID NO: 3; a CDR-L2 sequence comprising the amino acid sequence of SEQ ID NO: 5; and a CDR-L3 sequence comprising the amino acid sequence of SEQ ID NO: 7;
(v) a CDR-H1 sequence comprising the amino acid sequence of SEQ ID NO: 8; a CDR-H2 sequence comprising the amino acid sequence of SEQ ID NO: 10; a CDR-H3 sequence comprising the amino acid sequence of SEQ ID NO: 11; a CDR-L sequence comprising the amino acid sequence of SEQ ID NO: 3; a CDR-L2 sequence comprising the amino acid sequence of SEQ ID NO: 5; and a CDR-L3 sequence comprising the amino acid sequence of SEQ ID NO: 7;
(vi) a CDR-H sequence comprising the amino acid sequence of SEQ ID NO: 8; a CDR-H2 sequence comprising the amino acid sequence of SEQ ID NO: 10; a CDR-H3 sequence comprising the amino acid sequence of SEQ ID NO: 11; a CDR-L sequence comprising the amino acid sequence of SEQ ID NO: 3; a CDR-L2 sequence comprising the amino acid sequence of SEQ ID NO: 5; and a CDR-L3 sequence comprising the amino acid sequence of SEQ ID NO: 6;
(vii) a CDR-H1 sequence comprising the amino acid sequence of SEQ ID NO: 28; a CDR-H2 sequence comprising the amino acid sequence of SEQ ID NO: 31; a CDR-H3 sequence comprising the amino acid sequence of SEQ ID NO: 32; a CDR-L1 sequence comprising the amino acid sequence of SEQ ID NO: 22; a CDR-L2 sequence comprising the amino acid sequence of SEQ ID NO: 24; and a CDR-L3 sequence comprising the amino acid sequence of SEQ ID NO: 26;
(viii) a CDR-H1 sequence comprising the amino acid sequence of SEQ ID NO: 29; a CDR-H2 sequence comprising the amino acid sequence of SEQ ID NO: 31; a CDR-H3 sequence comprising the amino acid sequence of SEQ ID NO: 32; a CDR-L1 sequence comprising the amino acid sequence of SEQ ID NO: 23; a CDR-L2 sequence comprising the amino acid sequence of SEQ ID NO: 25; and a CDR-L3 sequence comprising the amino acid sequence of SEQ ID NO: 26; and
(ix) a CDR-H1 sequence comprising the amino acid sequence of SEQ ID NO: 30; a CDR-H2 sequence comprising the amino acid sequence of SEQ ID NO: 31; a CDR-H3 sequence comprising the amino acid sequence of SEQ ID NO: 32; a CDR-L1 sequence comprising the amino acid sequence of SEQ ID NO: 23; a CDR-L2 sequence comprising the amino acid sequence of SEQ ID NO: 25; and a CDR-L3 sequence comprising the amino acid sequence of SEQ ID NO: 26.

E5. The antibody of E1-E4, comprising a CDR-H1 sequence comprising the amino acid sequence of SEQ ID NO: 8; a CDR-H2 sequence comprising the amino acid sequence of SEQ ID NO: 10; a CDR-H3 sequence comprising the amino acid sequence of SEQ ID NO: 11; a CDR-L1 sequence comprising the amino acid sequence of SEQ ID NO: 1; a CDR-L2 sequence comprising the amino acid sequence of SEQ ID NO: 4; and a CDR-L3 sequence comprising the amino acid sequence of SEQ ID NO: 7.

E6. The antibody of E1-E4, comprising a CDR-H1 sequence comprising the amino acid sequence of SEQ ID NO: 9; a CDR-H2 sequence comprising the amino acid sequence of SEQ ID NO: 10; a CDR-H3 sequence comprising the amino acid sequence of SEQ ID NO: 12; a CDR-L1 sequence comprising the amino acid sequence of SEQ ID NO: 1; a CDR-L2 sequence comprising the amino acid sequence of SEQ ID NO: 4; and a CDR-L3 sequence comprising the amino acid sequence of SEQ ID NO: 7.

E7. The antibody of E1, comprising a CDR-H1 comprising an amino acid sequence comprising 0, 1 or 2 substitutions, deletions, or additions to the amino acid sequence of SEQ ID NO: 8; a CDR-H2 comprising an amino acid sequence comprising 0, 1 or 2 substitutions, deletions, or additions to the amino acid sequence of SEQ ID NO: 10; and a CDR-H3 comprising an amino acid sequence comprising 0, 1 or 2 substitutions, deletions, or additions to the amino acid sequence of SEQ ID NO: 11; a CDR-L1 comprising an amino acid sequence comprising 0, 1 or 2 substitutions, deletions, or additions to the amino acid sequence of SEQ ID NO: 1; a CDR-L2 comprising an amino acid sequence comprising 0, 1 or 2 substitutions, deletions, or additions to the amino acid sequence of SEQ ID NO: 4; and a CDR-L3 comprising an amino acid sequence comprising 0, 1 or 2 substitutions, deletions, or additions to the amino acid sequence of SEQ ID NO: 7.

E8. The antibody of any one of E1-E7, comprising one or more of the following substitutions: (i) 1, 2, 3, 4, 5, or 6 substitutions in CDRL1 to the corresponding residue of a human germline VL sequence,
(ii) 1, 2, 3, 4, or 5 substitutions in CDRL2 to the corresponding residue of a human VL germline sequence,
(iii) 1, 2, 3, 4, 5, or 6 substitutions in CDRL3 to the corresponding residue of a human germline VL sequence,
(iv) 1 substitution in CDRH1 to the corresponding residue of a human germline VH sequence,
(v) 1, 2, 3, 4, 5, 6, 7, or 8 substitutions in CDRH2 to the corresponding residue of a human germline VH sequence,
wherein the human germline VH is selected from the group consisting of IGHV3-7, (DP54) IGHV3-30 (DP49), IGHV3-23 (DP47), DP50, DP31, DP46, DP71, DP75, DP10, DP7, DP51, DP38, DP79, DP78, DP73, VH3, VH5, VH1, and VH4, and the human germline VL sequence is selected from the group consisting of IGKV3-15*01 (DPK21), IGKV3-11*01, IGKV3-20*01 (DPK22), DPK9, DPK12, DPK18, DPK24, HK102_V1, DPK1, DPK8, DPK3, Vg_38K, DPK15, DPL16, DPL8, V1-22, VX consensus, VA1 consensus, VX3 consensus, VK consensus, VK1 consensus, VK2 consensus, and VK3.

E9. The antibody of any one of E1-E8, comprising a MIGIS-α VH framework sequence derived from a human germline selected from the group consisting of IGHV3-7*01 (also known as DP-54) optionally further comprising a IGJH4*01 sequence; IGHV3-30*18 (also known as DP-49) optionally further comprising a IGHJ2*01 sequence, IGHV3-23*01 (also known as DP-47) optionally further comprising a IGHJ2*01 sequence, IGHV3-23*01 (also known as DP-47) optionally further comprising a IGJH4*01 sequence, IGHV3-7*01 (also known as DP-54) optionally further comprising a IGHJ2*01 sequence, and IGHV3-9*01.

E10. The antibody of anyone of E1-E9, comprising a MIGIS-α VH framework sequence derived from a human germline IGHV3-7*01 sequence, optionally further comprising a IGJH4*01 sequence.

E11. The antibody of any one of E1-E10, comprising a MIGIS-α VL framework sequence derived from a human germline selected from the group consisting of IGKV3-15*01 (also known as DPK21) optionally further comprising a IGKJ3*01 sequence, IGKV3-11*01 optionally further comprising a IGKJ3*01 sequence, and IGKV3-20*01 (also known as DPK22) optionally further comprising a IGKJ3*01 sequence.

E12. The antibody of any one of E1-E11, comprising a MIGIS-α VL framework sequence derived from a human germline IGKV3-15*01 sequence, optionally further comprising a human germline optionally further comprising a IGKJ3*01 sequence.

E13. The antibody of any one of E1-E12, comprising a VL framework sequence and a VH framework sequence, and wherein one or both of the VL framework sequence or VH framework sequence is at least 90%, 91%, 92%, 93%, 94%, 95%, 96%, 97%, 98%, 99%, or 100% identical to the human germline sequence from which it was derived.

E14. The antibody of any one of E1-E13, wherein the VH comprises the amino acid sequence of SEQ ID NO: 14 or a variant of SEQ ID NO: 14 thereof comprising one to four amino acid substitutions at residues that are not within a CDR, and the VL comprises the amino acid sequence of SEQ ID NO: 21 or a variant of SEQ ID NO: 21 thereof comprising one to four amino acid substitutions at residues that are not within a CDR.

E15. The antibody of any one of E1-E14, wherein the amino acid sequence of the VH is at least 90%, 95%, 98%, 99%, or 100% identical to the amino acid sequence of SEQ ID NO: 14, and the amino acid sequence of the VL is at least 90%, 95%, 98%, 99%, or 100% identical to the amino acid sequence of SEQ ID NO: 21 and wherein the VH comprises a CDR-H1 sequence comprising the amino acid sequence of SEQ ID NO: 1; a CDR-H2 sequence comprising the amino acid sequence of SEQ ID NO: 4; and a CDR-H3 sequence comprising the amino acid sequence of SEQ ID NO: 7; and wherein the VL comprises a CDR-L1 sequence comprising the amino acid sequence of SEQ ID NO: 8; a CDR-L2 sequence comprising the amino acid sequence of SEQ ID NO: 10, and a CDR-L3 sequence comprising the amino acid sequence of SEQ ID NO: 11.

E16. The antibody of any one of E1-E15, comprising a MIGIS-α VH comprising an amino acid sequence at least 90%, 91%, 92%, 93%, 94%, 95%, 96%, 97%, 98%, 99%, or 100% identical to SEQ ID NO: 14.

E17. The antibody of any one of E1-E16, comprising a MIGIS-α VH comprising an amino acid sequence of a sequence selected from the group consisting of SEQ ID NO: 14, SEQ ID NO: 15, SEQ ID NO: 18, SEQ ID NO: 19, SEQ ID NO: 20, SEQ ID NO: 35, and SEQ ID NO: 37, SEQ ID NO: 39, and SEQ ID NO: 41.

E18. The antibody of any one of E1-E17, comprising a MIGIS-α VH comprising an amino acid sequence of a sequence selected from the group consisting of SEQ ID NO: 14, SEQ ID NO: 15, SEQ ID NO: 18, SEQ ID NO: 19, and SEQ ID NO: 20.

E19. The antibody of any one of E1-E18, comprising a MIGIS-α VL comprising an amino acid sequence at least 90%, 91%, 92%, 93%, 94%, 95%, 96%, 97%, 98%, 99%, or 100% identical to SEQ ID NO: 21.

E20. The antibody of any one of E1-E19, comprising a MIGIS-α VL comprising an amino acid sequence of a sequence selected from the group consisting of SEQ ID NO: 21, SEQ ID NO: 33, SEQ ID NO: 34, SEQ ID NO: 38, SEQ ID NO: 40, and SEQ ID NO: 42.

E21. The antibody of any one of E1-E20, comprising a MIGIS-α VL comprising an amino acid sequence of a sequence selected from the group consisting of SEQ ID NO: 21, SEQ ID NO: 33, and SEQ ID NO: 34.

E22. The antibody of any one of E1-E21, comprising a VH comprising an amino acid sequence selected from the group consisting of SEQ ID NO: 14, SEQ ID NO: 15, SEQ ID NO: 18, SEQ ID NO: 19, and SEQ ID NO: 20, and comprising a VL comprising an amino acid sequence selected from the group consisting of SEQ ID NO: 21, SEQ ID NO: 33, and SEQ ID NO: 34.

E23. The antibody of any one of E1-E22, comprising a heavy chain variable region (VH) and a light chain variable region (VL) selected from the one of the groups in the following list:
(i) a VH comprising the amino acid sequence of SEQ ID NO: 14 and a VL comprising the amino acid sequence of 21;
(ii) a VH comprising the amino acid sequence of SEQ ID NO: 14 and a VL comprising the amino acid sequence of SEQ ID NO: 34;
(iii) a VH comprising the amino acid sequence of SEQ ID NO: 18 and a VL comprising the amino acid sequence of SEQ ID NO: 21; and
(iv) a VH comprising the amino acid sequence of SEQ ID NO: 15 and a VL comprising the amino acid sequence of SEQ ID NO: 21;
(v) a VH comprising the amino acid sequence of SEQ ID NO: 19 and a VL comprising the amino acid sequence of SEQ ID NO: 33;
(vi) a VH comprising the amino acid sequence of SEQ ID NO: 19 and a VL comprising the amino acid sequence of SEQ ID NO: 21;
(vii) a VH comprising the amino acid sequence of SEQ ID NO: 20 and a VL comprising the amino acid sequence of SEQ ID NO: 21; and
(viii) a VH comprising the amino acid sequence of SEQ ID NO: 41 and a VL comprising the amino acid sequence of SEQ ID NO: 42;
(ix) a VH comprising the amino acid sequence of SEQ ID NO: 39 and a VL comprising the amino acid sequence of SEQ ID NO: 40;
(x) a VH comprising the amino acid sequence of SEQ ID NO: 37 and a VL comprising the amino acid sequence of SEQ ID NO: 38; and
(xi) a VH comprising the amino acid sequence of SEQ ID NO: 35 and a VL comprising the amino acid sequence of SEQ ID NO: 36.

E24. The antibody of any one of E1-E23, comprising a heavy chain variable region (VH) and a light chain variable region (VL) selected from the one of the groups in the following list: (i) a VH comprising the amino acid sequence of SEQ ID NO: 14 and a VL comprising the amino acid sequence of SEQ ID NO: 21;
(ii) a VH comprising the amino acid sequence of SEQ ID NO: 14 and a VL comprising the amino acid sequence of SEQ ID NO: 34;
(iii) a VH comprising the amino acid sequence of SEQ ID NO: 18 and a VL comprising the amino acid sequence of SEQ ID NO: 21; and
(iv) a VH comprising the amino acid sequence of SEQ ID NO: 15 and a VL comprising the amino acid sequence of SEQ ID NO: 21;
(v) a VH comprising the amino acid sequence of SEQ ID NO: 19 and a VL comprising the amino acid sequence of SEQ ID NO: 33;
(vi) a VH comprising the amino acid sequence of SEQ ID NO: 19 and a VL comprising the amino acid sequence of SEQ ID NO: 21; and
(vii) a VH comprising the amino acid sequence of SEQ ID NO: 20 and a VL comprising the amino acid sequence of SEQ ID NO: 21.

E25. The antibody of any one of E1-24, comprising a VH sequence encoded by a polynucleotide comprising the nucleic acid sequence of SEQ ID NO: 17.

E26. The antibody of any one of E1-E25, comprising a VL sequence encoded by a polynucleotide comprising the nucleic acid sequence of SEQ ID NO: 16.

E27. The antibody of any one of E1-E26, wherein the MIGIS-α VL sequence is encoded by the plasmid deposited at the ATCC and having ATCC Accession No. PTA-127794.

E28. The antibody of any one of E1-E27, wherein the MIGIS-α VH sequence is encoded by the plasmid deposited at the ATCC and having ATCC Accession No. PTA-127796.

E29. The antibody of any one of E1-E28, wherein the antibody comprises an Fc domain, and the Fc domain is selected from the group consisting of IgG, IgG₁, IgG₂, IgG₃, IgG₄, IgA, IgA₁, IgA₂, IgD, IgE, and IgM,

E30. The antibody of any one of E1-E29, wherein the antibody comprises an Fc domain, and the Fc domain is an IgG.

E31. The antibody of any one of E1-E30, wherein the antibody comprises an Fc domain, and the Fc domain is an IgG₁.

E32. The antibody of anyone of E1-E31, comprising a constant heavy chain domain comprising an amino acid sequence at least 90%, 91%, 92%, 93%, 94%, 95%, 96%, 97%, 98%, 99%, or 100% identical to a sequence selected from the group consisting of SEQ ID NO: 43, SEQ ID NO: 44, SEQ ID NO: 46, SEQ ID NO: 68.

E33. The antibody of any one of E1-E32, comprising a heavy chain comprising an amino acid sequence at least 90%, 91%, 92%, 93%, 94%, 95%, 96%, 97%, 98%, 99%, or 100% identical to a sequence selected from the group consisting of SEQ ID NO: 48, SEQ ID NO: 40, SEQ ID NO: 50, SEQ ID NO: 52, SEQ ID NO: 54, SEQ ID NO: 56, SEQ ID NO: 69, SEQ ID NO: 71, and SEQ ID NO: 74.

E34. The antibody of any one of E1-E33, comprising a heavy chain comprising an amino acid sequence at least 90%, 91%, 92%, 93%, 94%, 95%, 96%, 97%, 98%, 99%, or 100% identical to SEQ ID NO: 48.

E35. The antibody of any one of E1-E34, comprising a heavy chain comprising an amino acid sequence of SEQ ID NO: 48.

E36. The antibody of any one of E1-E35, comprising a heavy chain that does not comprise a C-terminal lysine residue.

E37. The antibody of any one of E1-E36, comprising a light chain comprising an amino acid sequence at least 90%, 91%, 92%, 93%, 94%, 95%, 96%, 97%, 98%, 99%, or 100% identical to a sequence selected from the group consisting of SEQ ID NO: 51, and SEQ ID NO: 57.

E38. The antibody of any one of E1-E37, comprising a light chain comprising an amino acid sequence at least 90%, 91%, 92%, 93%, 94%, 95%, 96%, 97%, 98%, 99%, or 100% identical to SEQ ID NO: 57.

E39. The antibody of any one of E1-E34, comprising a light chain comprising an amino acid sequence of SEQ ID NO: 57.

E40. The antibody of E1-E39, comprising a heavy chain comprising an amino acid of SEQ ID NO: 48, and a light chain comprising an amino acid of SEQ ID NO: 57.

E41. The antibody of any one of E1-40, comprising a HC sequence encoded by a polynucleotide comprising the nucleic acid sequence of SEQ ID NO: 17.

E42. The antibody of any one of E1-E41, comprising a LC sequence encoded by a polynucleotide comprising the nucleic acid sequence of SEQ ID NO: 16.

E43. The antibody of any one of E1-E42, wherein the LC sequence is encoded by the plasmid deposited at the ATCC and having ATCC Accession No. PTA-127794.

E44. The antibody of any one of E1-E43, wherein the HC sequence is encoded by the plasmid deposited at the ATCC and having ATCC Accession No. PTA-127796.

E45. An isolated antibody, or antigen-binding fragment thereof, that competes for binding to human MIGIS-α with an antibody or antigen-binding fragment thereof of any one of E1-E44.

E46. An isolated antibody, or antigen-binding fragment thereof, that competes for binding to human MIGIS-α with one or more of GBT-IgA-1602, GBT-IgA-1449, GBT-IgA-1623, GBT-IgA-1470, GBT-IgA-1164, GBT-IgA-0862, GBT-IgA-0027, GBT-IgA-15E7, and GBT-IgA-H05.

E47. An isolated antibody, or antigen-binding fragment thereof, that competes for binding to human MIGIS-α with one or more of GBT-IgA-1602, and GBT-IgA-1449.

E48. The antibody of E1-E47, wherein the antibody binds to an epitope on human MIGIS-a located between residue M10 and residue L19, wherein the numbering is of SEQ ID NO: 78 (M18, L27 of SEQ ID NO: 84).

E49. The antibody of E48, wherein the antibody does not bind one or more of D7, W8, Q9, P20, or Q21 wherein the numbering is of SEQ ID NO: 78 (D15, W16, Q17, P28, Q29 of SEQ ID NO: 84).

E50. The antibody of E48 or E49, wherein the antibody does not bind two or more of D7, W8, Q9, P20, or Q21 wherein the numbering is of SEQ ID NO: 78 (D15, W16, Q17, P28, or Q29 of SEQ ID NO: 84).

E51. The antibody of any one of E48-E50, wherein the antibody does not bind three or more of D7, W8, Q9, P20, or Q21 wherein the numbering is of SEQ ID NO: 78 (D15, W16, Q17, P28, or Q29 of SEQ ID NO: 84).

E52. The antibody of any one of E48-E51, wherein the antibody does not bind four or more of D7, W8, Q9, P20, or Q21 wherein the numbering is of SEQ ID NO: 78 (D15, W16, Q17, P28, or Q29 of SEQ ID NO: 84).

E53. The antibody of any one of E48-E52, wherein the antibody does not bind any of D7, W8, Q9, P20, and Q21 wherein the numbering is of SEQ ID NO: 78 (D15, W16, Q17, P28, or Q29 of SEQ ID NO: 84).

E54. The antibody of any one of E48 to E53, wherein the antibody binds to an epitope on human MIGIS-α comprising one or more amino acid residues selected from the group consisting of M10, P11, P13, Y14, L17, D18, and L19 wherein the numbering is of SEQ ID NO: 78 (M18, P19, P21, Y22, L25, D26, and L27 of SEQ ID NO: 84).

E55. The antibody of any one of E48-E54, wherein the epitope comprises one or more amino acid residues selected from the group consisting of P11, L17, and L19 wherein the numbering is of SEQ ID NO: 78 (P19, L25, L27 of SEQ ID NO: 84).

E56. The antibody of any one of E48-E55, wherein the epitope on human MIGIS-α comprises two or more amino acid residues selected from the group consisting of P11, L17, and L19 wherein the numbering is of SEQ ID NO: 78 (P19, L25, L27 of SEQ ID NO: 84).

E57. The antibody of any one of E48-E56, wherein the epitope on human MIGIS-α comprises P11, L17, and L19 wherein the numbering is of SEQ ID NO: 78 (P19, L25, L27 of SEQ ID NO: 84).

E58. The antibody of any one of E48-E57, wherein the epitope on human MIGIS-α further comprises one or more of Y14 and wherein the numbering is of SEQ ID NO: 78 (Y22 of SEQ ID NO: 84).

E59. The antibody of any one of E48-E58, wherein the epitope on human MIGIS-α further comprises Y14 and D18 wherein the numbering is of SEQ ID NO: 78 (Y22 and D26 of SEQ ID NO: 84).

E60. The antibody of any one of E48-E59, wherein the epitope on human MIGIS-α further comprises one or more of Y14 and D18 wherein the numbering is of SEQ ID NO: 78 (Y22 and D26 of SEQ ID NO: 84).

E61. The antibody of any one of E48-E60, wherein the epitope on human MIGIS-α further comprises one or more of M10, P13, and V16 wherein the numbering is of SEQ ID NO: 78 (M18, P21, V24 of SEQ ID NO: 84).

E62. The antibody of any one of E48-E61, wherein the epitope on human MIGIS-α further comprises two or more of M10, P13, and V16 wherein the numbering is of SEQ ID NO: 78 (M18, P21, V24 of SEQ ID NO: 84).

E63. The antibody of any one of E48-E62, wherein the epitope on human MIGIS-α further comprises M10, P13, and V16 wherein the numbering is of SEQ ID NO: 78 (M18, P21, V24 of SEQ ID NO: 84).

E64. The antibody of any one of E48-E63, wherein the antibody binds to an epitope on human MIGIS-α comprising one or more amino acid residues selected from the group consisting of P11, Y14, L17, D18, and L19 wherein the numbering is of SEQ ID NO: 78 (P19, Y22, L25, D26, L27 of SEQ ID NO: 84).

E65. The antibody of any one of E48-E64, wherein the antibody binds to an epitope on human MIGIS-α comprising P11, Y14, L17, D18, and L19 wherein the numbering is of SEQ ID NO: 78 (P19, Y22, L25, D26, L27 of SEQ ID NO: 84).

E66. The antibody of any one of E48-E65, wherein the antibody binds to an epitope on human MIGIS-α comprising M10, P11, P13, Y14, L17, D18, and L19 wherein the numbering is of SEQ ID NO: 78 (M18, P19, P21, Y22, L25, D26, L27 of SEQ ID NO: 84).

E67. The antibody of E1-E66, wherein the antibody binds to an epitope on human MIGIS-α, and wherein the epitope is helical.

E68. The antibody of E1-E67, wherein the antibody binds to an epitope on human MIGIS-α, comprising P11, P13, Y14, V16, L17, D18, and L19, wherein the numbering is of SEQ ID NO: 78 (P19, P21, Y22, V24, L25, D26, and L27 of SEQ ID NO: 84).

E69. The antibody of E1-E68, wherein the antibody binds to a helical epitope on human MIGIS-a, comprising P11, P13, Y14, V16, L17, D18, and L19, wherein the numbering is of SEQ ID NO: 78 (P19, P21, Y22, V24, L25, D26, and L27 of SEQ ID NO: 84).

E70. The antibody of E1-E69, wherein the antibody binds to an epitope on MIGIS-α further comprising residue M10, wherein the numbering is of SEQ ID NO: 78 (M18 of SEQ ID NO: 84).

E71. The antibody of E1-E70, wherein the antibody binds to an epitope on MIGIS-α further comprising residue Q21, wherein the numbering is of SEQ ID NO: 78 (Q29 of SEQ ID NO: 84).

E72. The antibody of E1-E71, wherein the antibody binds to an epitope on human MIGIS-α, comprising M10, P11, P13, Y14, V16, L17, D18, L19, and Q21 wherein the numbering is of SEQ ID NO: 78 (M18, P19, P21, Y22, V24, L25, D26, L27, and Q29 of SEQ ID NO: 84).

E73. The antibody of E1-E72, wherein the antibody binds to a helical epitope on human MIGIS-a comprising P11, P13, Y14, V16, L17, D18, and L19, and further binds to residues M10 and Q21 on MIGIS-α, wherein the numbering is of SEQ ID NO: 78 (M18, P19, P21, Y22, V24, L25, D26, L27, and Q29 of SEQ ID NO: 84).

E74. The antibody of any one of E1-E73, comprising residue Y91 in the VL.

E75. The antibody of any one of E1-E74, comprising residue W94 in the VL.

E76. The antibody of any one of E1-E75, comprising residue T97 in the VL.

E77. The antibody of any one of E1-E76, comprising residue S31 in the VH.

E78. The antibody of any one of E1-E77, comprising residue Y32 in the VH.

E79. The antibody of any one of E1-E78, comprising residue G33 in the VH.

E80. The antibody of any one of E1-E79, comprising residue V50 in the VH.

E81. The antibody of any one of E1-E80, comprising residue S52 in the VH.

E82. The antibody of any one of E1-E81, comprising residue D53 in the VH.

E83. The antibody of any one of E1-E82, comprising residue Y101 in the VH.

E84. The antibody of any one of E1-E83, comprising residue H102 in the VH.

E85. The antibody of any one of E1-E84 comprising residue W103 in the VH.

E86. The antibody of any one of E1-E85, comprising a S, T, N, Q, R, K, H, D, E residue at position 31 of CDR-H1, an A or G residue at position 33 of CDR-H1, an S or T residue at position 52 of CDR-H2, Y58 in CDR-H2, G98 in CDR-H3, N92 in CDR-L3, and W94 in CDR-L3.

E87. The antibody of E1-E86, comprising H or S at position 31 of CDR-H1.

E88. The antibody of E1-E87, comprising H at position 31 of CDR-H1.

E89. The antibody of E1-E88, comprising G at position 33 of CDR-H1.

E90. The antibody of E1-E89, comprising S at position 52 of CDR-H2.

E91. The antibody of any one of E1-E90, comprising one or more residues selected from the group consisting of CDR-H3-G95, CDR-H3-G96, CDR-H3-N97, and CDR-H3-D100b.

E92. The antibody of any one of E1-E91, comprising CDR-H3-G95.

E93. The antibody of any one of E1-E92, comprising CDR-H3-G96.

E94. The antibody of any one of E1-E93, comprising CDR-H3-N97.

E95. The antibody of any one of E1-E94, comprising CDR-H3-D100b.

E96. The antibody of any one of E1-E95, comprising CDR-H3-G95, CDR-H3-G96, CDR-H3-N97, and CDR-H3-D100b.

E97. The antibody of any one of E1-E96, comprising CDR-H1-Y32.

E98. The antibody of any one of E1-E97, comprising CDR-H2-H56.

E99. The antibody of any one of E1-E98, comprising CDR-L3-Y91.

E100. The antibody of any one of E1-E99, comprising CDR-L3-F96.

E101. The antibody of any one of E1-E100, comprising CDR-H1-Y32, CDR-H2-H56; CDR-L3-Y91; and CDR-L3-F96.

E102. The antibody of E1-E101 comprising one or more residues selected from the group consisting of CDR-H2-A50, CDR-H2-G52a, CDR-H3-E95, CDR-H3-R96, CDR-H3-S97, CDR-L1-Y32, and CDR-L3-R91.

E103. The antibody of any one of E1-E102, comprising a MIGIS-α VH framework sequence derived from a human germline selected from the group consisting of IGHV3-7*01 (also known as DP-54) optionally further comprising a IGJH4*01 sequence; IGHV3-30*18 (also known as DP-49) optionally further comprising a IGHJ2*01 sequence, IGHV3-23*01 (also known as DP-47) optionally further comprising a IGHJ2*01 sequence, IGHV3-23*01 (also known as DP-47) optionally further comprising a IGJH4*01 sequence, IGHV3-7*01 (also known as DP-54) optionally further comprising a IGHJ2*01 sequence, and IGHV3-9*01.

E104. The antibody of any one of E1-E103, comprising a MIGIS-α VH framework sequence derived from a human germline IGHV3-7*01 sequence, optionally further comprising a IGJH4*01 sequence.

E105. The antibody of any one of E1-E104, comprising a MIGIS-α VL framework sequence derived from a human germline selected from the group consisting of IGKV3-15*01 (also known as DPK21) optionally further comprising a IGKJ3*01 sequence, IGKV3-11*01 optionally further comprising a IGKJ3*01 sequence, and IGKV3-20*01 (also known as DPK22) optionally further comprising a IGKJ3*01 sequence.

E106. The antibody of any one of E1-E105, comprising a MIGIS-α VL framework sequence derived from a human germline IGKV3-15*01 sequence, optionally further comprising a human germline optionally further comprising a IGKJ3*01 sequence.

E107. The antibody of any one of E1-E106, wherein the antibody binds to human MIGIS-α on the surface of B-cells expressed as the C-terminal membrane anchor of the a chain of mIgA.

E108. The antibody of any one of E1-E107, wherein the antibody binds human MIGIS-α with a K_{D} about or less than a value selected from the group consisting of about 100 nM, 50 nM, 20 nM, 10 nM, 5 nM, and 2.5 nM.

E109. The antibody of any one of E1-E108, wherein the antibody binds human MIGIS-α with a K_{D} about or less than a value of about 5 nM.

E110. The antibody of any one of E1-E109, wherein the antibody binds human MIGIS-α with a K_{D} about or less than a value of about 2.5 nM.

E111. The antibody of anyone of E1-E110, wherein the antibody the antibody binds cynomolgus MIGIS-α with a K_{D} about or less than a value selected from the group consisting of about 100 nM, 50 nM, 20 nM, 10 nM, 5 nM, and 2.5 nM.

E112. The antibody of anyone of E1-E111, wherein the antibody the antibody binds cynomolgus MIGIS-α with a K_{D} about or less than about 5 nM.

E113. The antibody of anyone of E1-E112, wherein the antibody the antibody binds cynomolgus MIGIS-α with a K_{D} about or less than about 2.55 nM.

E114. The antibody of any one of E108-E113, wherein the K_{D} value is measured by surface plasmon resonance (SPR).

E115. The antibody of any one of E108-E114, wherein the K_{D} value is measured by surface plasmon resonance (SPR), and the MIGIS-α is immobilized.

E116. An isolated antibody that specifically binds to human MIGIS-α and specifically binds to human CD3.

E117. The antibody of E1-E116, wherein the antibody further binds to human CD3.

E118. The antibody of any one of E1-E117, wherein the antibody or antigen-binding fragment is one or more of an Fc fusion protein, a monobody, a maxibody, a bispecific antibody, a bifunctional antibody, an scFab, an scFv, a peptibody.

E119. The antibody of any one of E1-E118, wherein the antibody is a bispecific antibody.

E120. The antibody of any one of E1-E119, wherein the antibody specifically binds to CD3 through a CD3 binding domain.

E121. The antibody of any one of E1-E120, wherein the antibody specifically binds to CD3 through a CD3 binding domain, comprising a CD3 binding heavy chain variable region (CD3-VH) and a CD3 binding light chain variable region (CD3-VL), comprising the CDRH1, CDRH2, and CDRH3 sequences of a CD3-VH sequence of SEQ ID NO: 66, and the CDRL1, CDRL2, and CDRL3 sequences of a CD3-VL sequence of SEQ ID NO: 67.

E122. The antibody of any one of E1-E121, wherein the antibody specifically binds to CD3 through a CD3 binding domain, comprising a CD3-binding heavy chain variable region (CD3-VH) and a CD3-binding light chain variable region (CD3-VL), wherein the CD3-VH comprises a CDRH1 sequence of SEQ ID NO: 58; a CDRH2 sequence of SEQ ID NO: 59; and a CDRH3 sequence of SEQ ID NO: 60; and the CD3-VL comprises a CDRL1 sequence of SEQ ID NO: 61; a CDRL2 sequence of SEQ ID NO: 62, and a CDRL3 sequence of SEQ ID NO: 63.

E123. The antibody of any one of E1-E122, wherein the CD3-VH comprises a CDRH1 sequence of SEQ ID NO: 58; a CDRH2 sequence of SEQ ID NO: 59; and a CDRH3 sequence of SEQ ID NO: 60; and the CD3-VL comprises a CDRL1 sequence of SEQ ID NO: 61; a CDRL2 sequence of SEQ ID NO: 62, and a CDRL3 sequence of SEQ ID NO: 63; and wherein the MIGIS-α-VH comprises a CDRH1 sequence of SEQ ID NO: 1; a CDRH2 sequence of SEQ ID NO: 4, and a CDRH3 sequence of SEQ ID NO: 7, and the MIGIS-α-VL comprises a CDRL1 sequence of SEQ ID NO: 8; a CDRL2 sequence of SEQ ID NO: 10, and a CDRL3 sequence of SEQ ID NO: 11.

E124. The antibody of any one of E120-E123, wherein CD3 binding domain comprises one or more of the following substitutions:
(i) 1, 2, 3, 4, 5, or 6 substitutions in CDRL1 to the corresponding residue of a human germline VL sequence,
(ii) 1, 2, 3, 4, or 5 substitutions in CDRL2 to the corresponding residue of a human VL germline sequence,
(iii) 1, 2, 3, 4, 5, or 6 substitutions in CDRL3 to the corresponding residue of a human germline VL sequence,
(iv) 1 substitution in CDRH1 to the corresponding residue of a human germline VH sequence,
(v) 1, 2, 3, 4, 5, 6, 7, or 8 substitutions in CDRH2 to the corresponding residue of a human germline VH sequence,
wherein the human germline VL sequence is selected from the group consisting of IGKV1-39, IGKV3-15*01, DPK9, DPK12, DPK18, DPK24, HK102_V1, DPK1, DPK8, DPK3, DPK21, Vg_38K, DPK22, DPK15, DPL16, DPL8, V1-22, Vλ consensus, Vλ1 consensus, Vλ3 consensus, Vκ consensus, Vκ1 consensus, Vκ2 consensus, and Vκ3, and the human germline VH is selected from the group consisting of IGHV3-7 HV3-30*18, DP54, DP47, DP50, DP31, DP46, DP71, DP75, DP10, DP7, DP49, DP51, DP38, DP79, DP78, DP73, VH3, VH5, VH1, and VH4.

E125. The antibody of any one of E120-E124, comprising a CD3 VH framework sequence derived from a human germline selected from the group consisting of IGHV3-7 sequence.

E126. The antibody of any one of E120-E125, comprising a CD3 VH framework sequence derived from a human germline IGHV3-7 sequence.

E127. The antibody of any one of E120-E126, comprising a CD3 VL framework sequence derived from a human germline selected from the group consisting of IGKV1-39 sequence.

E128. The antibody of any one of E120-E127, comprising a VL framework sequence derived from a human germline IGKV1-39 sequence.

E129. The antibody of any one of E120-E128, comprising a VL framework sequence and a VH framework sequence, and wherein one or both of the VL framework sequence or VH framework sequence is at least 90% identical to the human germline sequence from which it was derived.

E130. The antibody of any one of E120-E129, comprising a VL framework sequence and a VH framework sequence, and wherein one or both of the VL framework sequence or VH framework sequence is at least 90%, 91%, 92%, 93%, 94%, 95%, 96%, 97%, 98%, 99%, or 100% identical to the human germline sequence from which it was derived.

E131. The antibody of any one of E120-E130, comprising a CD3 VH comprising an amino acid sequence at least 90%, 91%, 92%, 93%, 94%, 95%, 96%, 97%, 98%, 99%, or 100% identical to SEQ ID NO: 66.

E132. The antibody of any one of E120-E131, comprising a CD3 VH comprising an amino acid sequence of SEQ ID NO: 66.

E133. The antibody of any one of E120-E132, comprising a CD3 VL comprising an amino acid sequence at least 90%, 91%, 92%, 93%, 94%, 95%, 96%, 97%, 98%, 99%, or 100% identical to SEQ ID NO: 67.

E134. The antibody of any one of E120-E133, comprising a CD3 VL comprising an amino acid sequence of SEQ ID NO: 67.

E135. The antibody of any one of E120-E134, comprising a CD3 VH comprising an amino acid sequence of SEQ ID NO: 66, and a CD3 VL comprising an amino acid sequence of SEQ ID NO: 67.

E136. The antibody of anyone of E120-E135, wherein the MIGIS-α VH comprises an amino acid sequence of SEQ ID NO: 14, the MIGIS-α VL comprises an amino acid sequence of SEQ ID NO: 21, the CD3 VH comprises an amino acid sequence of SEQ ID NO: 66, and the CD3 VL comprises an amino acid sequence of SEQ ID NO: 67.

E137. The antibody of any one of E120-E136, wherein the CD3 VH sequence is encoded by the plasmid deposited at the ATCC and having ATCC Accession No. PTA-127797.

E138. The antibody of any one of E91-E137, wherein the CD3 VL sequence is encoded by the plasmid deposited at the ATCC and having ATCC Accession No. PTA-127795.

E139. The antibody of any one of E1-E138, comprising a MIGIS-α VH sequence encoded by the plasmid deposited at the ATCC and having ATCC Accession No. PTA-127796, and a MIGIS-α VL sequence encoded by the plasmid deposited at the ATCC and having ATCC Accession No. PTA-127794, and that further binds to human CD3 through a CD3 binding domain comprising a CD3 variable heavy region (CD3 VH) and a CD3 variable light region (CD3 VL), comprising a CD3 VH sequence encoded by the plasmid deposited at the ATCC and having ATCC Accession No. PTA-127797, and a CD3 VL sequence encoded by the plasmid deposited at the ATCC and having ATCC Accession No. PTA-127795.

E140. An isolated antibody, or antigen-binding fragment thereof, that competes for binding to human MIGIS-α and human CD3 with an antibody or antigen-binding fragment thereof of any one of E125-E144.

E141. The antibody of any one of E120-E140, comprising an Fc domain.

E142. The antibody of any one of E1-E141, wherein the antibody comprises an Fc domain, and the Fc domain is selected from the group consisting of IgG, IgG₁, IgG₂, IgG₃, IgG₄, IgA, IgA₁, IgA₂, IgD, IgE, and IgM.

E143. The antibody of any one of E1-E142, wherein the antibody comprises an Fc domain, and the Fc domain is an IgG.

E144. The antibody of any one of E1-E143, wherein the antibody comprises an Fc domain, and the Fc domain is an IgG₁.

E145. The antibody of any one of E120-E144, comprising a heavy chain constant domain comprising an amino acid sequence at least 90%, 91%, 92%, 93%, 94%, 95%, 96%, 97%, 98%, 99%, or 100% identical to a sequence selected from the group consisting of SEQ ID NO: 43, SEQ ID NO: 44, SEQ ID NO: 46, SEQ ID NO: 68.

E146. The antibody of any one of E120-E145, comprising a CD3 heavy chain comprising an amino acid sequence at least 90%, 91%, 92%, 93%, 94%, 95%, 96%, 97%, 98%, 99%, or 100% identical to SEQ ID NO: 64.

E147. The antibody of any one of E120-E146, wherein the CD3 HC does not comprise a C-terminal lysine residue.

E148. The antibody of any one of E120-E147, comprising a CD3 LC comprising an amino acid sequence at least 90%, 91%, 92%, 93%, 94%, 95%, 96%, 97%, 98%, 99%, or 100% identical to SEQ ID NO: 65.

E149. The antibody of any one of E120-E148, comprising a CD3 LC comprising an amino acid according to SEQ ID NO: 65.

E150. The antibody of any one of E1-E149, comprising a MIGIS-α HC comprising an amino acid sequence of SEQ ID NO: 48, a MIGIS-α LC comprising an amino acid sequence of SEQ ID NO: 57, a CD3 HC comprising an amino acid sequence of SEQ ID NO: 64, and a CD3 LC comprising an amino acid sequence of SEQ ID NO: 65.

E151. The antibody of any one of E120-E150, comprising the CD3 HC sequence encoded by the plasmid deposited at the ATCC and having ATCC Accession No. PTA-127797.

E152. The antibody, or antigen-binding fragment thereof, of any one of E120-E151, comprising the CD3 LC sequence encoded by the plasmid deposited at the ATCC and having ATCC Accession No. PTA-127795.

E153. The antibody of any one of E1-E152, wherein the MIGIS-α binding domain comprises the HC sequence encoded by the plasmid deposited at the ATCC and having ATCC Accession No. PTA-127796, and the LC sequence encoded by the plasmid deposited at the ATCC and having ATCC Accession No. PTA-127794, and wherein the antibody further binds to CD3 through a CD3 binding domain, and the CD3 binding domain comprises the HC sequence encoded by the plasmid deposited at the ATCC and having ATCC Accession No. PTA-127797 and the LC sequence encoded by the plasmid deposited at the ATCC and having ATCC Accession No. PTA-127795.

E154. The antibody of any one of E1-E153, comprising a MIGIS-α-VH sequence encoded by a nucleic acid sequence of SEQ ID NO: 17.

E155. The antibody of any one of E1-E154, comprising a MIGIS-α-VL sequence encoded by a nucleic acid sequence of SEQ ID NO: 16.

E156. The antibody of any one of E1-E155, comprising a MIGIS-α-HC sequence encoded by a nucleic acid sequence of SEQ ID NO: 17.

E157. The antibody of any one of E1-E156, comprising a MIGIS-α-LC sequence encoded by a nucleic acid sequence of SEQ ID NO: 16.

E158. The antibody of any one of E1-E157, comprising a CD3-VH sequence encoded by a nucleic acid sequence of SEQ ID NO: 55.

E159. The antibody of any one of E1-E158, comprising a CD3-VL sequence encoded by a nucleic acid sequence of SEQ ID NO: 49.

E160. The antibody of any one of E1-E159, comprising a CD3-HC sequence encoded by a nucleic acid sequence of SEQ ID NO: 55.

E161. The antibody of any one of E1-E160, comprising a CD3-LC sequence encoded by a nucleic acid sequence of SEQ ID NO: 49.

E162. The antibody of E120, wherein the CD3 binding domain comprises a VH and VL domain comprising CDRs identical to the CDRs of an anti CD3 antibody selected from the group consisting of teplizumab, glofitamab, mosunetuzmab, epcoritamab, otelixizumab, visilizumab, foralumab, and muromonab.

E163. The antibody of E162, wherein the CD3 binding domain comprises a VH and VL domain at least 90%, 91%, 92%, 93%, 94%, 95%, 96%, 97%, 98%, 99%, or 100% identical to the VH and VL domain of an anti CD3 antibody selected from the group consisting of teplizumab, glofitamab, mosunetuzmab, epcoritamab, otelixizumab, visilizumab, foralumab, and muromonab.

E164. The antibody of E120, wherein the CD3 binding domain comprises a VH and VL domain identical to the VH and VL domain of an anti CD3 antibody selected from the group consisting of teplizumab, glofitamab, mosunetuzmab, epcoritamab, otelixizumab, visilizumab, foralumab, and muromonab.

E165. An isolated antibody that competes for binding to human MIGIS-α and with human CD3 with an antibody of any one of E1-E164.

E166. An isolated antibody that competes for binding to human MIGIS-α and human CD3 with one or more of GBT-IgA-1602, GBT-IgA-1623, and GBT-IgA-1164.

E167. An isolated antibody that competes for binding to human MIGIS-α and human CD3 with GBT-IgA-1602.

E168. The antibody of any one of E1-E167, wherein the antibody, or antigen binding fragment thereof, binds Ramos cells overexpressing human mIgA with an EC₅₀ about or less than a value selected from the group consisting of about 500 nM, 200 nM, 100 nM, 50 nM, and 40 nM.

E169. The antibody of any one of E1-E168, wherein the antibody, or antigen binding fragment thereof, binds Ramos cells overexpressing human mIgA with an EC₅₀ about or less than about 50 nM.

E170. The antibody of any one of E1-E169, wherein the antibody, or antigen binding fragment thereof, binds Ramos cells overexpressing human mIgA with an EC₅₀ about or less than about 40 nM.

E171. An isolated nucleic acid molecule, comprising one or more nucleotide sequences encoding the antibody of any one of E1-E170.

E172. An isolated nucleic acid molecule, comprising one or more nucleotide sequences encoding the antibody of any one of E1-E171, for use with one or more nucleotide sequences encoding the antibody of any one of E1-E171.

E173. An isolated nucleic acid molecule, comprising a MIGIS-α-HC sequence encoded by a nucleic acid sequence of SEQ ID NO: 16.

E174. An isolated nucleic acid molecule, comprising a MIGIS-α-LC sequence encoded by a nucleic acid sequence of SEQ ID NO: 17.

E175. An isolated nucleic acid molecule, comprising a MIGIS-α-HC sequence encoded by a nucleic acid sequence of SEQ ID NO: 16, and a MIGIS-α-LC sequence encoded by a nucleic acid sequence of SEQ ID NO: 17.

E176. An isolated nucleic acid molecule, comprising a MIGIS-α-HC sequence encoded by a nucleic acid sequence of SEQ ID NO: 16, for use with a MIGIS-α LC of SEQ ID NO: 57 or encoded by a nucleic acid sequence of SEQ ID NO: 17.

E177. An isolated nucleic acid molecule, comprising a MIGIS-α-LC sequence encoded by a nucleic acid sequence of SEQ ID NO: 17, for use with a MIGIS-α HC of SEQ ID NO: 48 or encoded by a nucleic acid sequence of SEQ ID NO: 16.

E178. A vector comprising one or more nucleic acid molecules as set forth in EE171-E177.

E179. A host cell comprising the nucleic acid molecule of any one of E171-E177, or the vector of E178.

E180. The host cell of E179, wherein said cell is a mammalian cell.

E181. The host cell of E180, wherein said host cell is a CHO cell, a HEK-293 cell, or an Sp2.0 cell.

E182. A method of making an antibody or antigen-binding fragment thereof, comprising culturing the host cell of E180 or E181, under a condition wherein said antibody or antigen-binding fragment is expressed by said host cell.

E183. The method of E182, further comprising isolating said antibody or antigen-binding fragment thereof.

E184. A pharmaceutical composition comprising an antibody or antigen-binding fragment thereof of any one of E1-E170, and a pharmaceutically acceptable carrier or excipient.

E185. A method of reducing the activity of IgA bound to B cells, comprising administering to a subject in need thereof a therapeutically effective amount of the antibody, or antigen-binding fragment thereof, of any one of embodiments E1-E170, or the pharmaceutical composition of E184.

E186. A method of treating an one or more conditions selected from the group consisting of IgA nephropathy (IgAN), Berger's disease, primary immunoglobulin A (IgA) nephropathy at risk of rapid disease progression, glomerular proteinuria, hematuria, inflammation of the glomeruli, glomerulonephritis, chronic glomerulonephritis, acute glomerulonephritis, Celiac Disease, and Henoch-Schonlein purpura, multiple myeloma (IgA subtype), IgA plasma cell neoplasms, Barrett's Esophagus, rheumatoid arthritis, COPD, Kawasaki Disease, IgA vasculitis, and idiopathic pulmonary fibrosis, comprising administering to a subject in need thereof a therapeutically effective amount of the antibody, or antigen-binding fragment thereof, of any one of E1-E170, or the pharmaceutical composition of E184.

E187. The method of E185 or E186, wherein said subject is a human.

E188. The method of anyone of E185-E187, comprising administering said antibody or antigen-binding fragment thereof, or pharmaceutical composition, intravenously.

E189. The method of anyone of E185-E187, comprising administering said antibody or antigen-binding fragment thereof, or pharmaceutical composition, subcutaneously.

E190. The method of any one of E185-189, wherein said antibody or pharmaceutical composition, is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, or once every four months.

E191. The antibody of any one of E1-E170, or the pharmaceutical composition of E184, for use as a medicament E192. The antibody of any one of E1-E170, or the pharmaceutical composition of E184, for use in reducing the activity of IgA in a subject.

E193. The antibody of any one of E1-E170, or the pharmaceutical composition of E184, for use in treating one or more conditions selected from the group consisting of IgA nephropathy (IgAN), Berger's disease, primary immunoglobulin A (IgA) nephropathy at risk of rapid disease progression, glomerular proteinuria, hematuria, inflammation of the glomeruli, glomerulonephritis, chronic glomerulonephritis, acute glomerulonephritis, Celiac Disease, and Henoch-Schonlein purpura, multiple myeloma (IgA subtype), IgA plasma cell neoplasms, Barrett's Esophagus, rheumatoid arthritis, COPD, Kawasaki Disease, IgA vasculitis, and idiopathic pulmonary fibrosis, in a subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Pfabat Numbering of exemplar sequences. Example numbering, with complementarity-determining regions (CDR) residues underlined in bold. For the heavy chain, the CDR definitions are: CDRH1 from residues H26-H35 (including insertion positions such as H35B); CDRH2 from residues H50-H65; and CDRH3 from residues H95-H102. Note that the definition of CDRH1 used herein includes positions H26-H29, which are not included in some other interpretations of Kabat numbering. For the light chain, the CDR definitions are: CDRL1 from residues L24-L34; CDRL2 from residues L50-L56; CDRL3 from residue L89-L97; FIG. 1 discloses SEQ ID NOS 86, 19, 87, 14, 66, 88, 21, 36, 21 and 67, respectively, in order of appearance (i.e., reading from left to right).

FIG. 2. Primary B cells and plasma cells isolated from donor PBMCs show low expression of membrane IgA. Membrane IgA receptor quantitation was determined as outlined in Example 1. Donor PBMCs and BMMNCs were stained with a PE labelled anti-IgA Fc antibody called 5F10 (internal Pfizer antibody). PE Quantibrite beads were used to determine the number of PE labelled 5F10 bound per cell by flow cytometry. In this example membrane IgA molecules present on primary memory B cells (IgA⁺, CD20⁺) and plasma cells (IgA⁺, CD38⁺, CD138⁺) were compared to membrane IgA overexpressing Ramos cell lines. IgA producing plasma cells in PBMCs and BMMNCs were estimated to have in the range of approximately 1,500-2,500 copies per cell respectively while memory B cells had approximately 5,300 copies per cell. CD20 is expressed on the surface of B-cells however expression is lost on terminally differentiated plasma cells.

FIG. 3. IgA Ramos cell binding characterization of prioritized affinity matured variants by Flow cytometry. IgA Ramos cell binding characterization of prioritized affinity matured variants by Flow cytometry (methods detailed in Example 2). HOS and optimized variants T18 and T25 show significant improvement in binding on IgA⁺ Ramos cells over the parental antibody HOS with left shift in EC50s and increased maximum mean fluorescence intensity (MFI) observed. Similarly optimized variants ET10, ET17 and ET18 also showed improved binding on IgA+ Ramos cells over parental 15E7.

FIG. 4. Binding characterization of antibodies T18 and c8G7 to Ramos cells overexpressing human IgA by Flow cytometry. GBT-IgA-0027 (afucosylated format) shows superior cell binding over c8G7 in afucosylated, effector enhanced and WT Fc (huIgG1) formats. MFI corresponds to the Mean Fluorescence Intensity.

FIG. 5A and FIG. 5B. Binding characterization of antibodies T18 and c8G7 to human IgA⁺ and IgD⁺ (IgA⁻) primary B cells by Flow cytometry in donor 35570 (FIG. 5A) and donor 35571 (FIG. 5B). FIG. 5A and FIG. 5B: BMCs were isolated from two healthy donors as per methods outlined in Example 5. T18 (GBT-IgA-0027) in afucosylated format shows superior cell binding over c8G7 in afucosylated, effector enhanced and WT Fc (huIgG1) formats. MFI corresponds to the Mean Fluorescence Intensity.

FIG. 6. Antibody dependent cellular cytotoxicity (ADCC) of primary B cells (CD19⁺, IgA⁺) with anti-MIGIS-α affinity matured variants. ADCC of primary B cells (CD19⁺, IgA⁺) with anti-MIGIS-α affinity matured variants 15E7 (ET10, ET17 and ET18) and HOS (T25 and T18) with enhanced effector function (S239D, I332E, A330L enhancing mutations huIgG1 Fc) showed poor cytotoxicity against primary B-cells isolated from donor PBMCs. Cell kill with T18 and T25 however was modestly increased over 15E7 and its optimized variants. Cytotoxicity was measured as % of cells that are co-positive for Annexin V and propidium iodide, subtracting the % measured in non-treated background control.

FIG. 7A and FIG. 7B. Sequence alignment of variable heavy and light chain amino acid sequences for anti-MIGIS-α binding arms derived from the HOS. FIG. 7A: Sequence alignment of variable heavy and light chain amino acid sequences for anti-MIGIS-α binding arms derived from the HOS clone identified from WyHN5 Naïve library panning in Example 2. HOS was subsequently optimized for affinity & developability characteristics delivering the final candidate molecule designated GBT-IgA-1179. Dots indicate shared amino acids at each position. Framework germlines are also shown. FIG. 7A discloses SEQ ID NOS 20, 19, 19, 18, 89, 90, 15, 14, 14, 21, 21, 33, 21, 21, 21, 21, 21, and 34, respectively, in order of appearance (i.e., reading from left to right). FIG. 7B: Identifiers for antibody formats are shown. Each row highlights entities with identical anti MIGIS-α variable regions variable regions. The nomenclature used for the Anti-CD3 epsilon binding arm is shown in column 5.

FIG. 8A and FIG. 8B. Alignments of the variable heavy and light chain amino acid sequences of T18, 15E7 and optimized variants of 15E7. FIG. 8A: Alignment of the variable heavy and light chain amino acid sequences of T18 and 15E7. Dots indicate identical amino acids shared with T18, dashes where no corresponding residues at that position. Framework germline identifiers are also shown. FIG. 8A discloses SEQ ID NOS 19, 35, 21, and 36, respectively, in order of appearance (i.e., reading from left to right). FIG. 8B: Alignment of variable heavy and light chain sequences of 15E7 and optimized variants (ET10, ET17 and ET18). FIG. 8B discloses SEQ ID NOS 35, 37, 39, 39, 36, 38, 40, and 42, respectively, in order of appearance (i.e., reading from left to right).

FIG. 9A, FIG. 9B, FIG. 9C, FIG. 9D, FIG. 9E. Graphics showing the structure of the T18/MIGIS-α complex (FIGS. 9A, 9B, 9C, 9D, 9E) and 15E7/MIGIS-α complex (FIG. 9E). FIG. 9A: shows the complex Structure of T18/MIGIS-α. T18 antibody shown in gray and MIGIS-α shown in black. All paratope/epitope residues are labeled and shown as sticks. FIG. 9B: Alignment of MIGIS-α domain with resolved peptide in crystal structure (str), DSSP categorization and SBD Epitope for T18 and 15E7. DSSP descriptions: H=alpha-helix, T=hydrogen bond turn, S=bend, −=random coil. FIG. 9C. Detail of Fab of GBT-IgA-0027 at 1.9 Å resolution structure binding to Long Form. FIG. 9D. Detail of Fab of GBT-IgA-0027 at 2.1 Å resolution structure binding to Short Form (2 copies). Cross-peptide interaction may be result of crystal contacts. Ribbon structure of the helical binding epitope is evident; rest of peptide & GS-linker are disordered. FIG. 9E. Comparison of T18/MIGIS-α complex (left panel) and 15E7/MIGIS-α complex (right panel), showing differences in antibody orientation relative to MIGIS-α helix.

FIG. 10. The reduction of secreted IgA in PBMCs supematants on treatment of depleting various antibody formats to drive IgA⁺ plasma cell kill. The mean reduction of secreted IgA in PBMCs supernatants on treatment of various antibody formats to drive IgA⁺ plasma cell depletion. Human IgA ELISA kit (Invitrogen) was used to quantitate soluble IgA in supernatants. Antibody formats (300 nM final conc.) were incubated with PBMCs in RPMI complete media for 72 hours at 37° C. in 5% CO₂. CD19-CD3 was incubated at 0.5 nM as positive control. GBT-IgA-0661 (T18-CD3 bispecific EFN EE/RR huIgG1) was the optimal format, reducing IgA levels to 52% in PBMC supernatants over untreated control, while CD19-CD3 treatment reduced IgA levels by 57%.

FIG. 11A, FIG. 11B and FIG. 11C. Exemplar cell based-binding data for GBT-IgA-1602. Experiments with cell lines were repeated three times (FIG. 11A and FIG. 11B). Binding to primary B cells (FIG. 11C) was assessed using three donors.

FIG. 12A and FIG. 12B. GBT-IgA-1602 induces selective cell killing of human soluble IgA producing cells within PBMCs. Collected supernatants from treated PBMCs were analyzed by ELISA for IgA (FIG. 12A) or IgM (FIG. 12B). Data represent mean and standard deviation of technical replicates from an exemplar experiment from 11 donors assessed. Dotted lines indicate the mean concentration of IgA or IgM measured from supernatants collected from untreated PBMCs.

FIG. 13A and FIG. 13B. Measured by flow cytometry, GBT-IgA-1602 induces selective cell killing of human mIgA⁺ memory B cells within human PBMCs. Exemplar experiment is shown from 15 donors assessed. Values represent single measurements of cytotoxicity based on cell counting of gated live singlet populations: IgA⁺ CD19⁺ IgD⁻ (FIG. 13A) or IgA⁻ CD19⁺ IgD⁻ (FIG. 13B).

FIG. 14. Table summarizing SEQ IDs relevant to selected anti-MIGIS-α antibodies (see also Table 2). OAA=one-armed antibody format. BiAb=bispecific antibody (with anti-CD3 antibody as described). MIGIS RR=monoclonal bivalent antibody comprising HC RR mutations.

### DETAILED DESCRIPTION

The section headings used herein are for organizational purposes only and are not to be construed as limiting the subject matter described.

All references cited herein, including patent applications, patent publications, UniProtKB accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al, Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds); Gene Transfer Vectors for Mammalian Cells (J. M. Miller and M. P. Calos, eds., 1987); PCR: The Polymerase Chain Reaction, (Mullis et al, eds., 1994); Current Protocols in Immunology (J. E. Coligan et al, eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999)); The Antibodies (M. Zanetti and J. D. Capra, eds., Harwood Academic Publishers, 1995); and updated versions thereof.

### Definitions

Unless otherwise defined herein, scientific and technical terms used in connection with the present invention have the meanings that are commonly understood by those of ordinary skill in the art.

As used herein, the singular form “a”, “an”, and “the” include plural references unless indicated otherwise. For example, “an” antibody includes one or more antibodies.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Any example(s) following the term “e.g.” or “for example” is not meant to be exhaustive or limiting.

As used herein, the term “about” when used to modify a numerically defined parameter means that the parameter may vary by as much as 10% below or above the stated numerical value for that parameter. For example, a dose of about 5 mg means 5%±10%, i.e. it may vary between 4.5 mg and 5.5 mg.

### Antibody

An “antibody” refers to an immunoglobulin molecule capable of specific binding to a target, such as a polypeptide, carbohydrate, polynucleotide, lipid, etc., through at least one antigen binding site, located in the variable region of the immunoglobulin molecule. As used herein, the term “antibody” can encompass any type of antibody (e.g. monospecific, bispecific), and includes portions of intact antibodies that retain the ability to bind to a given antigen (e.g. an “antigen-binding fragment”), and any other modified configuration of an immunoglobulin molecule that comprises an antigen binding site.

An antibody includes an antibody of any class, such as IgG, IgA, or IgM (or sub-class thereof), and the antibody need not be of any particular class. Depending on the antibody amino acid sequence of the constant region of its heavy chains (HC), immunoglobulins can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁ and IgA₂. The heavy chain constant regions that correspond to the different classes of immunoglobulins are called alpha, delta, epsilon, gamma, and mu, respectively. The subunit structures and three-dimensional configurations of different classes of immunoglobulins are well known.

Examples of antibody antigen-binding fragments and modified configurations include (i) a Fab fragment (a monovalent fragment consisting of the VL, VH, CL and CH1 domains); (ii) a F(ab′)2 fragment (a bivalent fragment comprising two Fab fragments linked by a disulfide bridge at the hinge region); and (iii) a Fv fragment consisting of the VL and VH domains of a single arm of an antibody. Furthermore, although the two domains of an Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)); see e.g., Bird et al., Science 1988; 242:423-426 and Huston et al., Proc. Natl. Acad. Sci. 1988 USA 85:5879-5883. Other forms of single chain antibodies, such as diabodies are also encompassed.

In addition, further encompassed are antibodies that are missing a C-terminal lysine (K) amino acid residue on a heavy chain polypeptide (e.g. human IgG1 heavy chain comprises a terminal lysine). As is known in the art, the C-terminal lysine is sometimes dipped during antibody production, resulting in an antibody with a heavy chain lacking the C-terminal lysine. Alternatively, an antibody heavy chain may be produced using a nucleic acid that does not include a C-terminal lysine.

### Variable Region

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J Mol Biol 196(4): 901-917, 1987).

In certain embodiments, definitive delineation of a CDR and identification of residues comprising the binding site of an antibody is accomplished by solving the structure of the antibody or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the AbM definition, the contact definition, the extended definition, and the conformational definition.

The Kabat definition is a standard for numbering the residues in an antibody and is typically used to identify CDR regions. See, e.g., Johnson & Wu, 2000, Nucleic Acids Res., 28: 214-8. The Chothia definition is similar to the Kabat definition, but the Chothia definition takes into account positions of certain structural loop regions. See, e.g., Chothia et al., 1986, J. Mol. Biol., 196: 901-17; Chothia et al., 1989, Nature, 342: 877-83. The extended definition is the combination of the Kabat and Chothia definitions. The AbM definition uses an integrated suite of computer programs produced by Oxford Molecular Group that model antibody structure. See, e.g., Martin et al., 1989, Proc Natl Acad Sci (USA), 86:9268-9272; “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any one or more of Kabat, Chothia, extended, AbM, contact, or conformational definitions.

### Pfabat Numbering Method Developed for Consistent Antibody Numbering

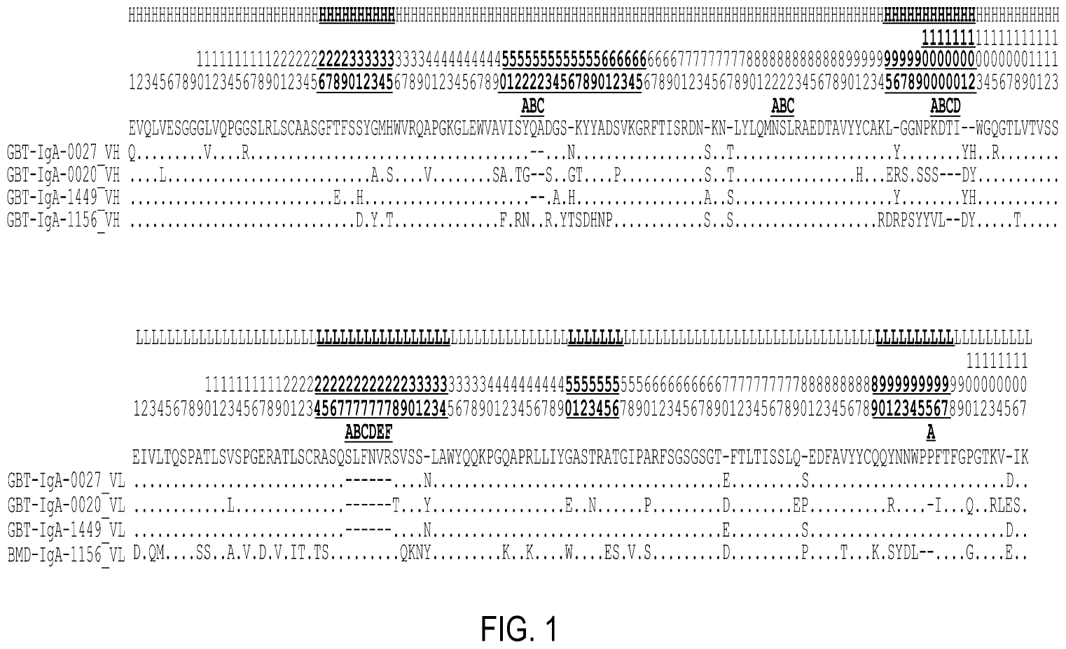

The Pfabat numbering method is a defined algorithm for consistent antibody numbering, based on the Kabat numbering system (Sequences of Proteins of Immunological Interest, Fifth Edition by Kabat et al., NIH Publication NO: 91-3242, 1991). Unlike many other computational implementations of Kabat numbering, Pfabat numbers entire human IgG1 heavy and light chains, including the constant (C) regions and heavy chain hinge. FIG. 1 shows example numbering, with complementarity-determining regions (CDR) residues underlined in bold. For the light chain, the CDR definitions are: CDRL1 from residues L24-L34; CDRL2 from residues L50-L56; CDRL3 from residue L89-L97; CDRH1 from residues H26-H35 (including insertion positions such as H35B); CDRH2 from residues H50-H65; and CDRH3 from residues H95-H102. Note that the definition of CDRH1 used herein includes positions H26-H29, which are not included in some other interpretations of Kabat numbering.

### Constant Region

A “constant region” of an antibody refers to the constant region of the antibody light chain or the constant region of the antibody heavy chain, either alone or in combination. An IgG heavy chain constant region contains three sequential immunoglobulin domains (CH1, CH2, and CH3), with a hinge region between the CH1 and CH2 domains. An IgG light chain constant region contains a single immunoglobulin domain (CL)

### Fc Domain and Fc Chain

A “Fc domain” refers to the portion of an immunoglobulin (Ig) molecule that correlates to a crystallizable fragment obtained by papain digestion of an Ig molecule. As used herein, the term relates to the 2-chained constant region of an antibody, each chain excluding the first constant region immunoglobulin domain. Within an Fc domain, there are two “Fc chains” (e.g. a “first Fc chain” and a “second Fc chain”). “Fc chain” generally refers to the C-terminal portion of an antibody heavy chain. Thus, Fc chain refers to the last two constant region immunoglobulin domains (CH2 and CH3) of IgA, IgD, and IgG heavy chains, and the last three constant region immunoglobulin domains of IgE and IgM heavy chains, and optionally the flexible hinge N-terminal to these domains.

Although the boundaries of the Fc chain may vary, the human IgG heavy chain Fc chain is usually defined to comprise residues C226 or P230 to its carboxyl-terminus, wherein the numbering is according to the EU index of Edelman et al., Proc. Natl. Acad. Sci. USA 1969; 63(1):78-85 and as described in Kabat et al., NIH publication No. 91-3242, Bethesda, 1991. Typically, the Fc chain comprises from about amino acid residue 236 to about 447 of the human IgG1 heavy chain constant region. “Fc chain” may refer to this polypeptide in isolation, or in the context of a larger molecule (e.g. in an antibody heavy chain or Fc fusion protein).

A “functional” Fc domain refers to an Fc domain that possesses at least one effector function of a native sequence Fc domain. Exemplary “effector functions” include C1q binding; complement dependent cytotoxicity (CDC); Fc receptor binding; antibody-dependent cell-mediated cytotoxicity (ADCC); phagocytosis; down-regulation of cell surface receptors (e.g., B cell receptor); and B cell activation, etc. Such effector functions generally require the Fc domain to be combined with a binding domain (e.g., an antibody variable region) and can be assessed using various assays known in the art for evaluating such antibody effector functions.

A “native sequence” Fc chain refers to a Fc chain that comprises an amino acid sequence identical to the amino acid sequence of an Fc chain found in nature. A “variant” Fc chain comprises an amino acid sequence which differs from that of a native sequence Fc chain by virtue of at least one amino acid modification.

### Monoclonal Antibody

A “monoclonal antibody” (mAb) refers to an antibody that is derived from a single copy or clone, including e.g., any eukaryotic, prokaryotic, or phage clone. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by the hybridoma method first described by Kohler and Milstein, 1975, Nature 256:495, or may be made by recombinant DNA methods such as described in U.S. Pat. No. 4,816,567. In another example, monoclonal antibodies may be isolated from phage libraries such as those generated using the techniques described in McCafferty et al., 1990, Nature 348:552-554.

### Human Antibody

A “human antibody” refers to an antibody which possesses an amino acid sequence which corresponds to that of an antibody produced by a human or has been made using any technique for making fully human antibodies. For example, fully human antibodies may be obtained by using commercially available mice that have been engineered to express specific human immunoglobulin proteins, or by library (e.g. phage, yeast, or ribosome) display techniques for preparing fully human antibodies. This definition of a human antibody specifically excludes a humanized antibody comprising non-human antigen binding residues.

### Chimeric Antibody

A “chimeric antibody” refers to an antibody in which the variable region sequences are derived from one species and the constant region sequences are derived from another species, such as an antibody in which the variable region sequences are derived from a mouse antibody and the constant region sequences are derived from a human antibody.

### Humanized Antibody

A “humanized” antibody refers to a non-human (e.g. murine) antibody that is a chimeric antibody that contains minimal sequence derived from non-human immunoglobulin. Preferably, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. The humanized antibody may comprise residues that are found neither in the recipient antibody nor in the imported CDR or framework sequences but are included to further refine and optimize antibody performance.

### Antigen

An “antigen” refers to the molecular entity used for immunization of an immunocompetent vertebrate to produce the antibody that recognizes the antigen or to screen an expression library (e.g., phage, yeast or ribosome display library, among others) for antibody selection. Herein, antigen is termed more broadly and is generally intended to include target molecules that are specifically recognized by the antibody, thus including fragments or mimics of the molecule used in an immunization process for raising the antibody or in library screening for selecting the antibody.

### Epitope

An “epitope” refers to the area or region of an antigen to which an antibody specifically binds, e.g., an area or region comprising residues that interact with the antibody, as determined by any method well known in the art. There are many methods known in the art for mapping and characterizing the location of epitopes on proteins, including solving the crystal structure of an antibody-antigen complex, competition assays, gene fragment expression assays, epitope mapping, and synthetic peptide-based assays, as described, for example, in Chapter 11 of Harlow and Lane, Using Antibodies, a Laboratory Manual, Cold Spring Harbor Laboratory Press, Cold Spring Harbor, New York, 1999. In addition or alternatively, during the discovery process, the generation and characterization of antibodies may elucidate information about desirable epitopes. From this information, it is then possible to competitively screen antibodies for binding to the same epitope.

In addition, the epitope to which an antibody binds can be determined in a systematic screening by using overlapping peptides derived from the antigen and determining binding by the antibody. According to the gene fragment expression assays, the open reading frame encoding the antigen can be fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis.

Certain epitopes can also be identified by using large libraries of random peptide sequences displayed on the surface of phage particles (phage libraries) or yeast (yeast display). Alternatively, a defined library of overlapping peptide fragments can be tested for binding to the test antibody in simple binding assays. In an additional example, mutagenesis of an antigen, domain swapping experiments and alanine scanning mutagenesis can be performed to identify residues required, sufficient, and/or necessary for epitope binding.

At its most detailed level, the epitope for the interaction between the antigen and the antibody can be defined by the spatial coordinates defining the atomic contacts present in the antigen-antibody interaction, as well as information about their relative contributions to the binding thermodynamics. At a less detailed level, the epitope can be characterized by the spatial coordinates defining the atomic contacts between the antigen and antibody. At a further less detailed level the epitope can be characterized by the amino acid residues that it comprises as defined by a specific criterion, e.g., by distance between atoms (e.g., heavy, i.e., non-hydrogen atoms) in the antibody and the antigen. At a further less detailed level the epitope can be characterized through function, e.g., by competition binding with other antibodies. The epitope can also be defined more generically as comprising amino acid residues for which substitution by another amino acid will alter the characteristics of the interaction between the antibody and antigen (e.g. using alanine scanning).

From the fact that descriptions and definitions of epitopes, dependent on the epitope mapping method used, are obtained at different levels of detail, it follows that comparison of epitopes for different antibodies on the same antigen can similarly be conducted at different levels of detail.

Epitopes described at the amino acid level, e.g., determined from an X-ray crystallography, Nuclear Magnetic Resonance (NMR) spectroscopy, hydrogen/deuterium exchange Mass Spectrometry (H/D-MS), are said to be identical if they contain the same set of amino acid residues. Epitopes are said to overlap if at least one amino acid is shared by the epitopes. Epitopes are said to be separate (unique) if no amino acid residue is shared by the epitopes.

Yet another method which can be used to characterize an antibody is to use competition assays with other antibodies known to bind to the same antigen, to determine if an antibody of interest binds to the same epitope as other antibodies. Competition assays are well known to those of skill in the art. Epitopes characterized by competition binding are said to be overlapping if the binding of the corresponding antibodies are mutually exclusive, i.e., binding of one antibody excludes simultaneous or consecutive binding of the other antibody. The epitopes are said to be separate (unique) if the antigen is able to accommodate binding of both corresponding antibodies simultaneously.

Epitopes can be linear or conformational. In a linear epitope, all of the points of interaction between the protein and the interacting molecule (such as an antibody) occur linearly along the primary amino acid sequence of the protein. A “nonlinear epitope” or “conformational epitope” comprises noncontiguous polypeptides (or amino acids) within the antigenic protein to which an antibody specific to the epitope binds.

### Binding Affinity

The term “binding affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art. Low-affinity antibodies generally bind antigen slowly and tend to dissociate readily, whereas high-affinity antibodies generally bind antigen faster and tend to remain bound longer. In particular, the term “binding affinity” is intended to refer to the dissociation rate of a particular antigen-antibody interaction. The K_{D} is the ratio of the rate of dissociation, also called the “off-rate (k_{off})” or “k_{d}” to the association rate, or “on-rate (kₒₙ)” or “kₐ”. Thus, K_{D} equals k_{off}/kₒₙ (or k_{d}/kₐ) and is expressed as a molar concentration (M). It follows that the smaller the K_{D}, the stronger the affinity of binding. Therefore, a K_{D} of 1 μM indicates weaker binding affinity compared to a K_{D} of 1 nM. K_{D} values for antibodies can be determined using methods well established in the art. One exemplary method for determining the K_{D} of an antibody is by using surface plasmon resonance (SPR), typically using a biosensor system such as BIACORE system. BIACORE kinetic analysis comprises analyzing the binding and dissociation of an antigen from chips with immobilized molecules (e.g., molecules comprising epitope binding domains), on their surface. Another method for determining the K_{D} of an antibody is by using Bio-Layer Interferometry, typically using OCTET® technology (Octet QK^{e} system, ForteBio). Alternatively, or in addition, a KinExA (Kinetic Exclusion Assay) assay, available from Sapidyne Instruments (Boise, ID) can also be used.

### Monospecific Antibody

A “monospecific antibody” refers to an antibody that comprises one or more antigen binding sites per molecule such that any and all binding sites of the antibody specifically recognize the identical epitope on the antigen. Thus, in cases where a monospecific antibody has more than one antigen binding site, the binding sites compete with each other for binding to one antigen molecule.

### Bispecific Antibody

A “bispecific antibody” refers to a molecule that has binding specificity for at least two different epitopes. In some embodiments, bispecific antibodies can bind simultaneously two different antigens. In other embodiments, the two different epitopes may reside on the same antigen.

### One-Armed Antibody

A one-armed antibody is an IgG antibody comprising an intact Fc domain of two hinge domains, two CH2 domain, and two CH3 domains, but only one Fab region. A one-armed antibody is monovalent and monospecific due to it possessing only a single Fab domain.

### Half Maximal Effective Concentration (EC₅₀)

The term “half maximal effective concentration (EC₅₀)” refers to the concentration of a therapeutic agent which causes a response halfway between the baseline and maximum after a specified exposure time. The therapeutic agent may cause inhibition or stimulation. The EC₅₀ value is commonly used, and is used herein, as a measure of potency.

### Agonist

An “agonist” refers to a substance which promotes (i.e., induces, causes, enhances, or increases) the biological activity or effect of another molecule. The term agonist encompasses substances (such as an antibody) which bind to a molecule to promote the activity of that molecule.

### Antagonist

An “antagonist” refers to a substance that prevents, blocks, inhibits, neutralizes, or reduces a biological activity or effect of another molecule, such as a receptor. The term antagonist encompasses substances (such as an antibody) which bind to a molecule to prevent or reduce the activity of that molecule.

### Compete

The term “compete”, as used herein with regard to an antibody, means that a first antibody binds to an epitope in a manner sufficiently similar to the binding of a second antibody such that the result of binding of the second antibody with its cognate epitope is detectably decreased in the presence of the first antibody compared to the binding of the second antibody in the absence of the first antibody. The alternative, where the binding of the first antibody to its epitope is also detectably decreased in the presence of the second antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing or cross-competing antibodies are encompassed and can be useful for the methods disclosed herein.

### Fc Receptor

An “Fc receptor” (FcR) refers to a receptor that binds to the Fc region of an antibody. In some embodiments, an FcR is a native human FcR. In some embodiments, an FcR is one which binds an IgG antibody (a gamma receptor) and includes receptors of the FcgRI, FcgRII, and FcgRIII subclasses, including allelic variants and alternatively spliced forms of those receptors. FcgRII receptors include FcgRIIA (an “activating receptor”) and FcgRIIB (an “inhibiting receptor”), which have similar amino acid sequences that differ primarily in the cytoplasmic domains thereof. Activating receptor FcgRIIA contains an immunoreceptor tyrosine-based activation motif (ITAM) in its cytoplasmic domain. Inhibiting receptor FcgRIIB contains an immunoreceptor tyrosine-based inhibition motif (ITIM) in its cytoplasmic domain, (see, e.g., Daeron, Annu. Rev. Immunol. 1997; 15:203-234). FcRs are reviewed, for example, in Ravetch and Kinet, Annu. Rev. Immunol 1991; 9:457-92; Capel et al., Immunomethods 1994; 4:25-34; and de Haas et al., J. Lab. Clin. Med. 1995; 126:330-41. Other FcRs, including those to be identified in the future, are encompassed by the term “Fc receptor” herein. The term “Fc receptor” also includes the neonatal receptor, FcRn, which is responsible for the transfer of maternal IgGs to the fetus (Guyer et al., J. Immunol. 1976; 117:587 and Kim et al., J. Immunol. 1994; 24:249) and regulation of homeostasis of immunoglobulins. Methods of measuring binding to FcRn are known (see, e.g., Ghetie and Ward., Immunol. Today 1997; 18(12):592-598; Ghetie et al., Nature Biotechnology, 1997; 15(7):637-640; Hinton et al., J. Biol. Chem. 2004; 279(8):6213-6216; WO 2004/92219).

### Effector Cells

An “effector cell” refers to a leukocyte which express one or more FcRs and performs effector functions. In certain embodiments, effector cells express at least FcgRIII and perform ADCC effector function(s). Examples of leukocytes which mediate ADCC include peripheral blood mononuclear cells (PBMC), natural killer (NK) cells, monocytes, macrophages, cytotoxic T cells, and neutrophils. Effector cells may be isolated from a native source, e.g., from blood.

### Antibody-Dependent Cell-Mediated Cytotoxicity (ADCC)

The term “antibody-dependent cell-mediated cytotoxicity” or “ADCC” refers to a form of cytotoxicity in which secreted Ig bound onto Fc receptors (FcRs) present on certain cytotoxic cells (e.g., NK cells, neutrophils, and macrophages) enable these cytotoxic effector cells to bind specifically to an antigen-bearing target cell and subsequently kill the target cell with cytotoxins. The primary cells for mediating ADCC, NK cells, express FcgRIII only, whereas monocytes express FcgRI, FcgRII, and FcgRIII. To assess ADCC activity of a molecule of interest, an in vitro ADCC assay, such as that described in U.S. Pat. Nos. 5,500,362, 5,821,337 or 6,737,056, may be performed. Useful effector cells for such assays include PBMC and NK cells. Alternatively, or additionally, ADCC activity of the molecule of interest may be assessed in vivo, e.g., in an animal model such as that disclosed in Clynes et al., Proc. Natl. Acad. Sci. (USA) 1998; 95:652-656. Additional antibodies with altered Fc region amino acid sequences and increased or decreased ADCC activity are described, e.g., in U.S. Pat. Nos. 7,923,538, and 7,994,290.

### Enhanced ADCC Activity

The term “enhanced ADCC activity” refers to an antibody that is more effective at mediating ADCC in vitro or in vivo compared to the parent antibody, wherein the antibody and the parent antibody differ in at least one structural aspect, and when the amounts of such antibody and parent antibody used in the assay are essentially the same. In some embodiments, the antibody and the parent antibody have the same amino acid sequence, but the antibody is afucosylated while the parent antibody is fucosylated. In some embodiments, ADCC activity will be determined using an in vitro ADCC assay, but other assays or methods for determining ADCC activity, e.g. in an animal model etc., are contemplated. In some embodiments, an antibody with enhanced ADCC activity has enhanced affinity for FcgRIIIA.

### Altered FcR Binding or ADCC Activity

The term “altered” FcR binding affinity or ADCC activity refers to an antibody which has either enhanced or diminished FcR binding activity and/or ADCC activity compared to a parent antibody, wherein the antibody and the parent antibody differ in at least one structural aspect. An antibody that “displays increased binding” to an FcR binds at least one FcR with better affinity than the parent antibody. An antibody that “displays decreased binding” to an FcR, binds at least one FcR with lower affinity than a parent antibody. Such antibodies that display decreased binding to an FcR may possess little or no appreciable binding to an FcR, e.g., 0-20 percent binding to the FcR compared to a native sequence IgG Fc region.

### Complement Dependent Cytotoxicity (CDC)

The term “Complement dependent cytotoxicity” or “CDC” refers to the lysis of a target cell in the presence of complement. Activation of the classical complement pathway is initiated by the binding of the first component of the complement system (Clq) to antibodies (of the appropriate subclass), which are bound to their cognate antigen. To assess complement activation, a CDC assay, e.g., as described in Gazzano-Santoro et al., J. Immunol. Methods 1996; 202: 163, may be performed. Antibodies with altered Fc region amino acid sequences and increased or decreased Clq binding capability are described, e.g., in U.S. Pat. Nos. 6,194,551, 7,923,538, 7,994,290 and WO 1999/51642.

### Host Cell

A “host cell” refers to an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

### Vector

A “vector” refers to a construct, which is capable of delivering, and, preferably, expressing, one or more gene(s) or sequence(s) of interest (e.g. an antibody-encoding gene) in a host cell. Examples of vectors include, but are not limited to plasmids and viral vectors, and may include naked nucleic acids, or may include nucleic acids associated with delivery-aiding materials (e.g. cationic condensing agents, liposomes, etc.). Vectors may include DNA or RNA. An “expression vector” as used herein refers to a vector that includes at least one polypeptide-encoding gene, at least one regulatory element (e.g. promoter sequence, poly(A) sequence) relating to the transcription or translation of the gene. Typically, a vector used herein contains at least one antibody-encoding gene, as well as one or more of regulatory elements or selectable markers. Vector components may include, for example, one or more of the following: a signal sequence; an origin of replication; one or more marker genes; suitable transcriptional controlling elements (such as promoters, enhancers and terminator). For translation, one or more translational controlling elements may also be included such as ribosome binding sites, translation initiation sites, and stop codons.

### Isolated

An “isolated” molecule (e.g. antibody) refers to a molecule that by virtue of its origin or source of derivation (1) is not associated with naturally associated components that accompany it in its native state, (2) is substantially free of other molecules from the same source, e.g., species, cell from which it is expressed, library, etc., (3) is expressed by a cell from a different species, or (4) does not occur in nature. Thus, a molecule that is chemically synthesized, or expressed in a cellular system different from the system from which it naturally originates, will be “isolated” from its naturally associated components. A molecule also may be rendered substantially free of naturally associated components by isolation, using purification techniques well known in the art.

### Polypeptide/Protein

A “polypeptide” or “protein” (used interchangeably herein) refers to a chain of amino acids of any length. The chain may be linear or branched. The chain may comprise one or more of modified amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

### Polynucleotide/Nucleic Acid

A “polynucleotide” or “nucleic acid,” (used interchangeably herein) refers to a chain of nucleotides of any length and includes DNA and RNA. The nucleotides can be deoxyribonucleotides, ribonucleotides, modified nucleotides or bases or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside.

### Conservative Substitution

A “conservative substitution” refers to replacement of one amino acid by a biologically, chemically or structurally similar residue. Biologically similar means that the substitution does not destroy a biological activity. Structurally similar means that the amino acids have side chains with similar length, such as alanine, glycine and serine or a similar size. Chemical similarity means that the residues have the same charge or are both hydrophilic or hydrophobic. Particular examples include the substitution of a hydrophobic residue, such as isoleucine, valine, leucine or methionine with another, or the substitution of one polar residue for another, such as the substitution of arginine for lysine, glutamic acid for aspartic acid or glutamine for asparagine, serine for threonine, and the like. Particular examples of conservative substitutions include the substitution of a hydrophobic residue such as isoleucine, valine, leucine or methionine for one another, the substitution of a polar residue for another, such as the substitution of arginine for lysine, glutamic acid for aspartic acid, or glutamine for asparagine, and the like. Conservative amino acid substitutions typically include, for example, substitutions within the following groups: glycine, alanine, valine, isoleucine, leucine; aspartic acid, glutamic acid; asparagine, glutamine; serine, threonine; lysine, arginine; and phenylalanine, tyrosine.

### Identity

The term “identity” or “identical to” refers to the overall relatedness between polymeric molecules, e.g., between nucleic acid molecules (e.g., DNA molecules or RNA molecules) or between polypeptide molecules. “Identity” measures the percent of identical matches between two or more sequences with gap alignments addressed by a particular mathematical model of computer programs (e.g. algorithms), which are well known in the art.

The terms “increase,” improve,” “decrease” or “reduce” refer to values that are relative to a baseline measurement, such as a measurement in the same individual prior to initiation of treatment described herein, or a measurement in a control individual or subject (or multiple control individuals or subjects) in the absence of the treatment described herein. In some embodiments, a “control individual” is an individual afflicted with the same form of disease or injury as an individual being treated. In some embodiments, a “control individual” is an individual that is not afflicted with the same form of disease or injury as an individual being treated.

### Excipient

The term ‘excipient’ refers to any material which, which combined with an active ingredient of interest (e.g. antibody), allow the active ingredient to retain biological activity. The choice of excipient will to a large extent depend on factors such as the mode of administration, the effect of the excipient on solubility and stability, and the nature of the dosage form. As used herein, “excipient” includes any and all solvents, dispersion media, coatings, antibacterial and antifungal agents, isotonic and absorption delaying agents, carriers, diluents and the like that are physiologically compatible. Examples of an excipient include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition.

### Treating

The terms “treating”, “treat” or “treatment” refer to any type of treatment, e.g. such as to relieve, alleviate, or slow the progression of the patient's disease, disorder or condition or any tissue damage associated with the disease. In some embodiments, the disease, disorder or condition is one or more selected from the group consisting of inflammatory bowel disease (IBD), Crohn's disease, ulcerative colitis, fibrostenosing Crohn's disease, irritable bowel syndrome, allergies, ankylosing spondylitis, alopecia areata, arthritis, asthma, atherosclerosis, atopic dermatitis, autoimmune hepatitis, autoimmune thyroiditis, Behcet's disease, bladder syndrome/intersticial cystitis, cutaneous lupus erythematosus, diabetes mellitus, eczematous dermatitis, encephalomyelitis, eosinophilic esophagitis, eosinophilic gastroenteritis, graft-versus-host disease (GVHD), idiopathic pulmonary fibrosis, juvenile rheumatoid arthritis, multiple sclerosis, myasthenia gravis, nonalcoholic fatty liver disease, nonalcoholic steatohepatitis, non-responsive celiac disease, osteoarthritis, primary biliary cirrhosis, primary sclerosing cholangitis, psoriasis, psoriatic arthritis, rheumatoid arthritis, scleroderma, sepsis, Sjogren's syndrome, spondyloarthropathy, systemic lupus erythematosus, systemic sclerosis with interstitial lung disease (SSc-ILD), transplant rejection, ulcerative proctitis, urinary bowel disfunction, uveitis, and vasculitis.

### Prevent

The terms “prevent” or “prevention” refer to one or more of delay of onset, reduction in frequency, or reduction in severity of at least one sign or symptom of a particular disease, disorder or condition. In some embodiments, prevention is assessed on a population basis such that an agent is considered to “prevent” a particular disease, disorder or condition if a statistically significant decrease in the development, frequency or intensity of one or more symptoms of the disease, disorder or condition is observed in a population susceptible to the disease, disorder or condition. Prevention may be considered complete when onset of disease, disorder or condition has been delayed for a predefined period of time.

### Subject

The terms “subject, “individual” or “patient,” (used interchangeably herein), refer to any animal, including mammals. Mammals according to the invention include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development. In some embodiments, a subject is a patient with one or more diseases or disorders selected from the group consisting of IgA nephropathy (IgAN), Berger's disease, primary immunoglobulin A (IgA) nephropathy at risk of rapid disease progression, glomerular proteinuria, hematuria, inflammation of the glomeruli, glomerulonephritis, chronic glomerulonephritis, acute glomerulonephritis, Celiac Disease, and Henoch-Schonlein purpura, multiple myeloma (IgA subtype), IgA plasma cell neoplasms, Barrett's Esophagus, rheumatoid arthritis, COPD, Kawasaki Disease, IgA vasculitis, and idiopathic pulmonary fibrosis.

### Therapeutically Effective Amount

The term “therapeutically effective amount” refers to the amount of active ingredient that elicits the biological or medicinal response in a tissue, system, animal, individual or human that is being sought by a researcher, veterinarian, medical doctor or other clinician, which may include one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting or slowing further development of the pathology or symptomatology); and
(3) ameliorating the disease; for example, ameliorating a disease, condition or disorder in an individual that is experiencing or displaying the pathology or symptomatology of the disease, condition or disorder (i.e., reversing the pathology or symptomatology).

### Antibodies to MIGIS-α

Membrane-bound IgA (mIgA) is associated with Igα/Igβ as the B cell receptor (BCR) complex on mIgA-expressing B cells. The alpha chain of mIgA contains a C-terminal membrane-anchor peptide, which encompasses extracellular, transmembrane and intracellular segments. The extracellular segment, referred to as the mIg isotype-specific (MIGIS-α) segment or the extracellular membrane proximal domain of the alpha chain, has been proposed to be a specific antigenic site suitable for isotype-specific targeting of mIgA-expressing B cells by antibodies.

The disclosure provides antibodies that bind to MIGIS-α (membrane immunoglobulin isotype-specific) domain. MIGIS-α is a 26-32 residue extracellular sequence unique to the membrane isoform of IgA (mIgA) and is not found on soluble IgA or other cell surface immunoglobulins. MIGIS-α is encoded by the IGHA1 and IGHA2 genes and is expressed as part of a functional B cell receptor on soluble IgA producing cells and memory B cell precursors.

As used herein, the term MIGIS-α includes variants, isoforms, homologs, orthologs and paralogs of MIGIS-α. For example, the term specifically encompasses both the predominant peptide form shared with human IgHA1 and IgHA2 (456C isoform: SEQ ID NO: 78), as well as a SNP variant (456S isoform: SEQ ID NO: 79). In some embodiments, an antibody disclosed herein cross-reacts with MIGIS-α from species other than human, such as MIGIS-α of cynomolgus monkey, as well as different forms of MIGIS-α. In some embodiments, an antibody may be completely specific for human MIGIS-α and may not exhibit species cross-reactivity (e.g., does not bind mouse or rat MIGIS-α). As used herein the term MIGIS-α refers to naturally occurring human MIGIS-α unless contextually dictated otherwise. Therefore, an “MIGIS-α antibody” “anti-MIGIS-α antibody” or other similar designation means any antibody (as defined herein) that binds or reacts with MIGIS-α, an isoform, fragment or derivative thereto.

The present disclosure provides a therapeutic strategy to target the hypothesized root cause of IgAN by selectively eliminating the production of circulating IgA, including Gd-IgA, which drives kidney damage. The MIGIS-α antibodies and MIGIS-α binding arm of MIGIS-α/CD3 bispecific antibodies described herein selectively binds an extracellular sequence unique to the membrane isoform of IgA, which is expressed on both IgA class switched memory B cells and soluble IgA producing cells.

Surprisingly, the present disclosure provides that concomitant use of a T cell retargeting antibody domain, in particular anti-CD3, significantly potentiates depletion of soluble IgA producing cells, and is more advantageous than other modalities tested. It is hypothesized that this is due to insufficient expression of mIgA on soluble IgA producing cells to support ADCC, CDC or ADCP. The CD3 binding domain induces T cell retargeting and cytotoxicity of MIGIS-α bearing cells, thereby depleting production of IgA antibodies that enter circulation. In vitro experiments using primary cells from healthy adults or IgAN patients show MIGIS-α/CD3 bispecific antibodies have selective cell killing potency against IgA producing cells.

Preliminary results of a comparison of GBT-IgA-1602 and a clinical anti-APRIL monoclonal antibody demonstrated that both induced significant and sustained decreases in serum IgA (data not shown). However, in contrast to the anti-APRIL antibody, GBT-IgA-1602 did not decrease serum IgM and IgG. The anti-MIGIS-α/CD3 bispecific antibodies described herein have the potential to be a transformative therapy against IgAN with its first-in-class mechanism for selectively depleting serum IgA levels.

Anti-MIGIS-α/CD3 bispecific antibodies of the disclosure directly and selectively kill soluble IgA producing cells. A selective IgA deficiency induced by anti-MIGIS-α/CD3 bispecific antibodies of the disclosure is likely a much safer approach for the long-term treatment of the root cause of IgAN. It is unproven that all IgA producing cells rely on APRIL for survival. It remains possible that with a deeper depletion of IgA producing cells with anti-MIGIS-α/CD3 bispecific antibodies of the disclosure, afforded by their direct targeting, the entire population of pathogenic Gd-IgA producing cells can be eliminated.

In some embodiments, an anti-MIGIS-α antibody of the disclosure encompasses an antibody that competes for binding to human MIGIS-α with, and/or binds the same epitope as, an antibody having the amino acid sequence of a heavy chain variable region set forth as SEQ ID NO: 14 and the amino acid sequence of a light chain variable region set forth as SEQ ID NO: 21.

In some embodiments, an anti-MIGIS-α antibody of the disclosure encompasses an antibody that competes for binding to human MIGIS-α with, and/or binds the same epitope as, an antibody having the amino acid sequence of a heavy chain variable region set forth as SEQ ID NO: 14 and the amino acid sequence of a light chain variable region set forth as SEQ ID NO: 34.

Anti-MIGIS-α antibodies of the present disclosure can encompass monoclonal antibodies, polyclonal antibodies, antibody fragments (e.g., Fab, Fab′, F(ab′)₂, Fv, Fc, etc.), chimeric antibodies, bispecific antibodies, heteroconjugate antibodies, single chain (ScFv), mutants thereof, fusion proteins comprising an antibody fragment (e.g., a domain antibody), humanized antibodies, and any other modified configuration of the immunoglobulin molecule that comprises an antigen binding site of the required specificity, including glycosylation variants of antibodies, amino acid sequence variants of antibodies, and covalently modified antibodies. The antibodies may be murine, rat, human, or any other origin (including chimeric or humanized antibodies). In some embodiments, an anti-MIGIS-α antibody is a monoclonal antibody. In some embodiments, an anti-MIGIS-α antibody is a human or humanized antibody. In some embodiments, an anti-MIGIS-α antibody is a chimeric antibody.

The invention also provides CDR portions of antibodies to MIGIS-α. In some embodiments, the antibody comprises three CDRs of any one of the heavy chain variable regions shown in Table 2. In some embodiments, the antibody comprises three CDRs of any one of the light chain variable regions shown in Table 2. In some embodiments, the antibody comprises three CDRs of any one of the heavy chain variable regions shown in Table 2 and three CDRs of any one of the light chain variable regions shown in Table 2. In some embodiments, the antibody comprises three light chain CDRs and three heavy chain CDRs from Table 2.

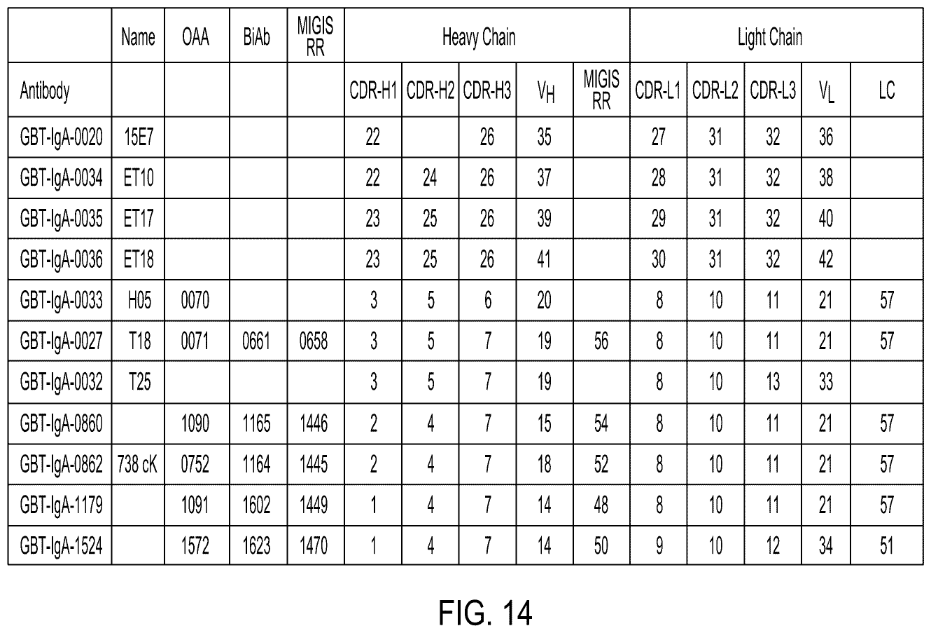

The invention also provides CDR portions of antibodies to MIGIS-α. In some embodiments, the antibody comprises three CDRs of any one of the heavy chain variable regions shown in FIG. 14. In some embodiments, the antibody comprises three CDRs of any one of the light chain variable regions shown in FIG. 14. In some embodiments, the antibody comprises three CDRs of any one of the heavy chain variable regions shown in FIG. 14 and three CDRs of any one of the light chain variable regions shown in FIG. 14. In some embodiments, the antibody comprises three light chain CDRs and three heavy chain CDRs from FIG. 14.

In some embodiments, the antibody comprises the full-length heavy chain, with or without the C-terminal lysine, and/or the full-length light chain of anti-MIGIS-α antibody GBT-IgA-1449 or GBT-IgA-1602. The amino acid sequences of the full-length heavy chain and light chain for antibodies GBT-IgA-1449 and GBT-IgA-1602 are shown in Table 2.

In certain embodiments, an antibody described herein comprises an Fc domain. The Fc domain can be derived from IgA (e.g., IgA₁ or IgA₂), IgG, IgE, or IgG (e.g., IgG₁, IgG₂, IgG₃, or IgG₄). In some embodiments, an anti-MIGIS-α antibody is an IgG1 antibody.

The invention encompasses modifications to the variable regions shown in Table 2, the CDRs shown in Table 2, and heavy chain and light chain sequences shown in Table 2. For example, the invention includes antibodies comprising functionally equivalent variable regions and CDRs which do not significantly affect their properties as well as variants which have enhanced or decreased activity and/or affinity. For example, the amino acid sequence may be mutated to obtain an antibody with the desired binding affinity to MIGIS-α. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand, or use of chemical analogs.

A modification or mutation may also be made in a framework region or constant region to increase the half-life of an antibody provided herein. See, e.g., WO00/09560. A mutation in a framework region or constant region can also be made to alter the immunogenicity of the antibody, to provide a site for covalent or non-covalent binding to another molecule, or to alter such properties as complement fixation, FcR binding and antibody-dependent cell-mediated cytotoxicity. In some embodiments, no more than one to five conservative amino acid substitutions are made within the framework region or constant region. In other embodiments, no more than one to three conservative amino acid substitutions are made within the framework region or constant region. According to the invention, a single antibody may have mutations in any one or more of the CDRs or framework regions of the variable domain or in the constant region.

In some embodiments, the antibody comprises a modified constant region that has increased or decreased binding affinity to a human Fc gamma receptor, is immunologically inert or partially inert, e.g., does not trigger complement mediated lysis, does not stimulate antibody-dependent cell mediated cytotoxicity (ADCC), or does not activate microglia; or has reduced activities (compared to the unmodified antibody) in any one or more of the following: triggering complement mediated lysis, stimulating ADCC, or activating microglia. Different modifications of the constant region may be used to achieve optimal level and/or combination of effector functions. See, for example, Morgan et al., Immunology 86:319-324, 1995; Lund et al., J. Immunology 157:4963-9 157:4963-4969, 1996; Idusogie et al., J. Immunology 164:4178-4184, 2000; Tao et al., J. Immunology 143: 2595-2601, 1989; and Jefferis et al., Immunological Reviews 163:59-76, 1998. In some embodiments, the constant region is modified as described in Eur. J. Immunol., 1999, 29:2613-2624; PCT Publication No. WO99/058572.

Modifications also include glycosylated and nonglycosylated polypeptides, as well as polypeptides with other post-translational modifications, such as, for example, glycosylation with different sugars, acetylation, and phosphorylation. Antibodies are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Jefferis and Lund, supra; Wyss and Wagner, 1996, Current Opin. Biotech. 7:409-416). Oligosaccharides may also serve to target a given glycoprotein to certain molecules based upon specific recognition structures. Glycosylation of antibodies has also been reported to affect antibody-dependent cellular cytotoxicity (ADCC). In particular, antibodies produced by CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., 1999, Nature Biotech. 17:176-180).

In some embodiments, the disclosure provides anti-MIGIS-α antibodies containing variations of the variable regions shown in Table 2, the CDRs shown in Table 2, and/or heavy chain and light chain sequences shown in Table 2, wherein such variant polypeptides share at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% amino acid sequence identity to any of the amino acid sequences disclosed in Table 2. These amounts are not meant to be limiting and increments between the recited percentages are specifically envisioned as part of the disclosure.

In some embodiments provided herein is an anti-MIGIS-α antibody that comprises a heavy chain and a light chain, wherein the antibody heavy chain has an amino acid sequence encoded by the nucleic acid sequence of the insert of the plasmid deposited with the ATCC having ATCC Accession No. PTA-127796 and the antibody light chain has an amino acid sequence encoded by the nucleic acid sequence of the insert of the plasmid deposited with the ATCC having ATCC Accession No. PTA-127794.

The invention also encompasses fusion proteins comprising one or more components of the antibodies disclosed herein. In some embodiments, a fusion protein may be made that comprises all or a portion of an anti-MIGIS-α antibody of the invention linked to another polypeptide. In another embodiment, only the variable domains of the anti-MIGIS-α antibody are linked to the polypeptide. In another embodiment, the VH domain of an anti-MIGIS-α antibody is linked to a first polypeptide, while the VL domain of an anti-MIGIS-α antibody is linked to a second polypeptide that associates with the first polypeptide in a manner such that the VH and VL domains can interact with one another to form an antigen binding site. In another embodiment, the VH domain is separated from the VL domain by a linker such that the VH and VL domains can interact with one another. The VH-linker-VL antibody is then linked to the polypeptide of interest. In addition, fusion antibodies can be created in which two (or more) single-chain antibodies are linked to one another. This is useful if one wants to create a divalent or polyvalent antibody on a single polypeptide chain, or if one wants to create a bispecific antibody.

### Biological Activity of Anti-MIGIS-α Antibodies

In addition to binding an epitope on MIGIS-α, an antibody of the disclosure can mediate a biological activity. That is, the disclosure includes an isolated antibody that specifically binds MIGIS-α and mediates at least one detectable activity selected from the following:
(i) binds specifically to human MIGIS-α;
(ii) binds specifically to cynomolgus monkey MIGIS-α;
(iii) initiates cell death in a population of IgA expressing cells.

In some embodiments, the disclosure provides polynucleotides encoding the amino acid sequences of any of the following anti-MIGIS-α antibodies: GBT-IgA-1449, GBT-IgA-1602 (also referred to as 1602), GBT-IgA-0033 (also referred to as HOS), GBT-IgA-0027 (also referred to as T18), GBT-IgA-0862, GBT-IgA-1164, GBT-IgA-1470, GBT-IgA-1623, and GBT-IgA-0020 (also referred to as 15E7). In one embodiment, the invention provides polynucleotides encoding the amino acid sequence of anti-MIGIS-α antibody GBT-IgA-1602. In one embodiment, the invention provides polynucleotides encoding the amino acid sequence of anti-MIGIS-α antibody GBT-IgA-1449.

The following examples of specific aspects for carrying out the present invention are offered for illustrative purposes only and are not intended to limit the scope of the present invention in any way.

The foregoing description and following Examples detail certain specific embodiments of the disclosure and describes the best mode contemplated by the inventors. It will be appreciated, however, that no matter how detailed the foregoing may appear in text, the disclosure may be practiced in many ways and the disclosure should be construed in accordance with the appended claims and any equivalents thereof.

Although the disclosed teachings have been described with reference to various applications, methods, kits, and compositions, it will be appreciated that various changes and modifications can be made without departing from the teachings herein and the claimed disclosure below. The following examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

The antibodies of the disclosure are designed to overcome a potential drawback of other B-cell targeting therapeutic agents, such as rituximab, which target the whole B cell population, (potentially increasing the risk of critical adverse effects or undesirable side effects). Further, although rituximab has shown promise as a drug that decreases the serum sIgA1 level through the depletion of CD20-positive cells, which are the precursor of plasma cell, it does not exhibit this beneficial effect in IgAN patients and did not reduce the serum sIgA1 level in IgAN patients. The antibodies of the disclosure are designed to overcome these drawbacks.

### Nucleic Acids

The disclosure also provides polynucleotides encoding any of the antibodies of the invention, including antibody portions and modified antibodies described herein. The invention also provides a method of making any of the antibodies and polynucleotides described herein. Polynucleotides can be made, and the proteins expressed, by procedures known in the art.

If desired, an antibody (monoclonal or polyclonal) of interest may be sequenced and the polynucleotide sequence may then be cloned into a vector for expression or propagation. The sequence encoding the antibody of interest may be maintained in vector in a host cell and the host cell can then be expanded and frozen for future use. Production of recombinant monoclonal antibodies in cell culture can be carried out through cloning of antibody genes from B cells by means known in the art. See, e.g. Tiller et al., 2008, J. Immunol. Methods 329, 112; U.S. Pat. No. 7,314,622.

In some embodiments, provided herein is a polynucleotide comprising a sequence encoding one or both of the heavy chain or the light chain variable regions of an antibody provided herein. The sequence encoding the antibody of interest may be maintained in a vector in a host cell and the host cell can then be expanded and frozen for future use. Vectors (including expression vectors) and host cells are further described herein.

In some embodiments, the disclosure provides polynucleotides encoding the amino acid sequences of any of the antibodies listed in Table 2. The polynucleotide may be RNA. The polynucleotide may comprise at least one chemical modification. The chemical modification may be selected from the group consisting of pseudouridine, 1-methylpseudouridine. N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, and 2′-O-methyl uridine.

In some aspects, the polynucleotide does not comprise a chemical modification.

In some aspects, there is provided a vector comprising one or more of the polynucleotides herein provided.

In some aspects, there is provided an isolated host cell comprising one or more the polynucleotides herein provided, or the vectors herein provided.

In some aspects, there is provided a method of producing an isolated antibody, comprising culturing the host cell of as herein provided under conditions that result in production of the antibody, and recovering the antibody.

In some aspects, there is provided a pharmaceutical composition comprising a therapeutically effective amount of the antibody as herein provided, and a pharmaceutically acceptable carrier.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions or substitutions of nucleotides. The resulting mRNA and protein may, but need not, have an altered structure or function. Alleles may be identified using standard techniques (such as hybridization, amplification or database sequence comparison).

In one embodiment, the VH and VL domains or full-length HC or LC, are encoded by separate polynucleotides. Alternatively, both VH and VL, or HC and LC, are encoded by a single polynucleotide.

Polynucleotides complementary to any such sequences are also encompassed by the present disclosure. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present disclosure, and a polynucleotide may, but need not, be linked to other molecules or support materials.

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome.

Suitable cloning vectors may be constructed according to standard techniques, or may be selected from a large number of cloning vectors available in the art. While the cloning vector selected may vary according to the host cell intended to be used, useful cloning vectors will generally have one or more features such as i) the ability to self-replicate, ii) a single target for a particular restriction endonuclease, or iii) may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors are further provided. Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The invention also provides host cells comprising any of the polynucleotides described herein. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis).

Additionally, any number of commercially and non-commercially available cell lines that express polypeptides or proteins may be utilized in accordance with the present invention. One skilled in the art will appreciate that different cell lines might have different nutrition requirements or might require different culture conditions for optimal growth and polypeptide or protein expression and will be able to modify conditions as needed.

### Pharmaceutical Compositions

In other embodiments, the invention comprises pharmaceutical compositions.

A “pharmaceutical composition” refers to a mixture of an antibody the invention and one or excipient. As used herein, pharmaceutical compositions may comprise one or more antibodies that bind to MIGIS-α, and/or MIGIS-α/CD3, or one or more polynucleotides comprising sequences encoding one or more these antibodies. These compositions may further comprise suitable excipients, such as pharmaceutically acceptable excipients including buffers, which are well known in the art.

Pharmaceutical compositions of this invention may be in a variety of forms. These include, for example, liquid, semi-solid and solid dosage forms, such as liquid solutions (e.g., injectable and infusible solutions), dispersions or suspensions, and lyophilized powders. The form depends on the intended mode of administration and therapeutic application.

Other excipients and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pennsylvania, 1975; Uberman et al., Eds., Pharmaceutical Dosage Forms, Marcel Decker, New York, N.Y., 1980; and Kibbe et al., Eds., Handbook of Pharmaceutical Excipients (3rd Ed.), American Pharmaceutical Association, Washington, 1999.

Acceptable excipients are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); or non-ionic surfactants such as TWEEN™, PLURONICS™ or polyethylene glycol (PEG).

The invention provided herein further encompasses methods and compositions for treatment, prevention or management of one or more disorders or diseases selected from the group consisting of IgA nephropathy (IgAN), Berger's disease, primary immunoglobulin A (IgA) nephropathy at risk of rapid disease progression, glomerular proteinuria, hematuria, inflammation of the glomeruli, glomerulonephritis, chronic glomerulonephritis, acute glomerulonephritis, Celiac Disease, and Henoch-Schonlein purpura, multiple myeloma (IgA subtype), IgA plasma cell neoplasms, Barrett's Esophagus, rheumatoid arthritis, COPD, Kawasaki Disease, IgA vasculitis, and idiopathic pulmonary fibrosis.

### Administration and Dosing

Typically, an antibody of the invention is administered in an amount effective to treat a condition as described herein. The antibodies the invention can be administered as an antibody per se, or alternatively, as a pharmaceutical composition containing the antibody.

The antibodies of the invention are administered by any suitable route in the form of a pharmaceutical composition adapted to such a route, and in a dose effective for the treatment intended.

In some embodiments, the antibodies may be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors, and infusion techniques.

In some aspects, MIGIS-α antibodies of the invention are administered subcutaneously. In some aspects, MIGIS-α, and/or MIGIS-α/CD3 antibodies of the invention are administered subcutaneously.

In another embodiment, the compounds of the invention may also be administered topically to the skin or mucosa, that is, dermally or transdermally. In another embodiment, the compounds of the invention can also be administered intranasally or by inhalation. In another embodiment, the compounds of the invention may be administered rectally or vaginally. In another embodiment, the compounds of the invention may also be administered directly to the eye or ear.

The dosage regimen for the antibodies of the invention or compositions containing said antibodies is based on a variety of factors, including the type, age, weight, sex and medical condition of the subject; the severity of the condition; the route of administration; and the activity of the particular antibody employed.

### Co-Administration

The antibodies of the invention can be used alone, or in combination with one or more other therapeutic agents. The invention provides any of the uses, methods or compositions as defined herein wherein an antibody of the invention is used in combination with one or more other therapeutic agent discussed herein.

The administration of two or more agents “in combination” means that all of the agents are administered closely enough in time to affect treatment of the subject. The two or more agents may be administered simultaneously or sequentially. Additionally, simultaneous administration may be carried out by mixing the agents prior to administration or by administering the agents at the same point in time but as separate dosage forms at the same or different site of administration.

Various formulations of the antibodies of the present invention (e.g., one or more of anti-MIGIS-α, and/or MIGIS-α/CD3 antibodies) may be used for administration. In some embodiments, the antibodies may be administered neat. In some embodiments, the antibody and a pharmaceutically acceptable excipient may be in various formulations. Pharmaceutically acceptable excipients are known in the art and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 21st Ed. Mack Publishing, 2005.

In some embodiments, these agents are formulated for administration by injection (e.g., intraperitoneally, intravenously, subcutaneously, intramuscularly, etc.). Accordingly, these agents can be combined with pharmaceutically acceptable vehicles such as saline, Ringer's solution, dextrose solution, and the like. The particular dosage regimen, i.e., dose, timing and repetition, will depend on the particular individual and that individual's medical history.

The antibodies (e.g., one or more of MIGIS-α, and/or MIGIS-α/CD3 antibodies) as described herein can be administered using any suitable method, including by injection (e.g., intraperitoneally, intravenously, subcutaneously, intramuscularly, etc.). The antibody, e.g., monoclonal antibody or multispecific antibody, also be administered via inhalation, as described herein. Generally, for administration of the antibody of the present, the dosage depends upon the host treated and the particular mode of administration. For repeated administrations over several days or longer, depending on the condition, the treatment is sustained until a desired suppression of symptoms occurs or until sufficient therapeutic levels are achieved.

For the purpose of the present invention, the appropriate dosage of an antibody (e.g., one or more selected from the group consisting of MIGIS-α, and/or MIGIS-α/CD3 antibodies) will depend on the antibody or compositions thereof employed, the type and severity of symptoms to be treated, whether the agent is administered for therapeutic purposes, previous therapy, the patient's clinical history and response to the agent, the patient's clearance rate for the administered agent, and the discretion of the attending physician. Typically, the clinician will administer an antibody until a dosage is reached that achieves the desired result. Dose and/or frequency can vary over course of treatment. Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of symptoms. Alternatively, sustained continuous release formulations of antibodies may be appropriate. Various formulations and devices for achieving sustained release are known in the art.

In one embodiment, dosages for an antibody (e.g., one or more selected from the group consisting of MIGIS-α, and/or MIGIS-α/CD3 antibodies) may be determined empirically in individuals who have been given one or more administration(s) of the antibody. Individuals are given incremental dosages of an antibody. To assess efficacy, an indicator of the disease can be followed.

In some embodiments, an antibody provided herein (e.g., one or more selected from the group consisting of MIGIS-α, and/or MIGIS-α/CD3 antibodies) may be administered to a subject that has previously received one or more antibodies selected from the group consisting of MIGIS-α, and/or MIGIS-α/CD3 antibody therapeutic for treatment of a disease. In some embodiments, an antibody provided herein may be an administered to a subject that has previously received an antibody selected from the group consisting of MIGIS-α, and/or MIGIS-α/CD3 antibody therapeutic for treatment of a disease, and for which the previous MIGIS-α, and/or MIGIS-α/CD3 antibody therapeutic is of limited or no efficacy in the subject (e.g. for which the subject's disease is resistant to treatment with the prior therapeutic).

Administration of an antibody in accordance with the method in the present invention can be continuous or intermittent, depending, for example, upon the recipient's physiological condition, whether the purpose of the administration is therapeutic or prophylactic, and other factors known to skilled practitioners. The administration of an antibody may be essentially continuous over a preselected period of time or may be in a series of spaced doses.

Therapeutic formulations of the antibody used in accordance with the present invention are prepared for storage by mixing an antibody having the desired degree of purity with optional pharmaceutically acceptable carriers, excipients or stabilizers (Remington, The Science and Practice of Pharmacy 21st Ed. Mack Publishing, 2005), in the form of lyophilized formulations or aqueous solutions. Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

### Kits

Another aspect of the invention provides kits comprising the antibody of the invention or pharmaceutical compositions comprising the antibody. A kit may include, in addition to the antibody of the invention or pharmaceutical composition thereof, diagnostic or therapeutic agents. A kit may also include instructions for use in a diagnostic or therapeutic method. In some embodiments, the kit includes the antibody or a pharmaceutical composition thereof and a diagnostic agent. In other embodiments, the kit includes the antibody or a pharmaceutical composition thereof and one or more therapeutic agents.

A further aspect of the invention is a kit comprising one or more selected from the group consisting of MIGIS-α, and/or MIGIS-α/CD3 antibodies as disclosed herein above and instructions for use in accordance with any of the methods of the invention described herein. Generally, these instructions comprise a description of administration one or more selected from the group consisting of MIGIS-α, and/or MIGIS-α/CD3 antibodies for the above described therapeutic treatments.

In yet another embodiment, the invention comprises kits that are suitable for use in performing the methods of treatment described herein. In one embodiment, the kit contains a first dosage form comprising one or more of the antibodies of the invention in quantities sufficient to carry out the methods of the invention. In another embodiment, the kit comprises one or more antibodies of the invention in quantities sufficient to carry out the methods of the invention and at least a first container for a first dosage and a second container for a second dosage.

Several aspects of the pharmaceutical compositions, prophylactic, or therapeutic agents of the invention are preferably tested in vitro, in a cell culture system, and in an animal model organism, such as a rodent animal model system, for the desired therapeutic activity prior to use in humans.

Toxicity and efficacy of the prophylactic and/or therapeutic protocols of the instant invention maybe determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it may be expressed as the ratio LD₅₀/ED₅₀. Prophylactic and/or therapeutic agents that exhibit large therapeutic indices are preferred.

Further, any assays known to those skilled in the art may be used to evaluate the prophylactic and/or therapeutic utility of the therapies or combinatorial therapies disclosed herein for treatment or prevention of cancer.

The instructions relating to the use of one or more selected from the group consisting of MIGIS-α, and/or MIGIS-α/CD3 antibodies as described herein generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, ampules, tubes, bottles, jars, flexible packaging (e.g., sealed Mylar or plastic bags), and the like for each pharmaceutical composition and other included reagents, e.g., buffers, balanced salt solutions, etc., for use in administering the pharmaceutical compositions to subjects. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition selected from the group consisting of MIGIS-α, and/or MIGIS-α/CD3 antibodies. The container may further comprise a second pharmaceutically active agent.

Normally, the kit comprises a container and a label or package insert(s) on or associated with the container.

Incorporated by reference herein for all purposes is the content of U.S. Provisional Patent Application Nos. 62/949,120 (filed Dec. 17, 2019) and 63/110,693 (filed Nov. 6, 2020).

As used herein, “mammalian cells” include reference to cells derived from mammals including humans, rats, mice, hamsters, guinea pigs, chimpanzees, or macaques. The cells may be cultured in vivo or in vitro.

As used herein, the term “purified product” refers to a preparation of the product which has been isolated from the cellular constituents with which the product is normally associated or from other types of cells that may be present in the sample of interest.

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

The term “non-human animals” of the invention includes all non-human vertebrates, e.g., non-human mammals and non-mammals, such as non-human primates, sheep, dog, cow, chickens, amphibians, reptiles, mouse, rat, rabbit or goat etc., unless otherwise noted.

As used herein, the term “pharmaceutically acceptable” refers to a product or compound approved (or approvable) by a regulatory agency of the Federal government or a state government or listed in the U.S. Pharmacopeia or other generally recognized pharmacopeia for use in animals, including humans.

As used herein, the terms “pharmaceutically acceptable excipient, carrier or adjuvant” or “acceptable pharmaceutical carrier” refer to an excipient, carrier or adjuvant that can be administered to a subject, together with at least one antibody of the present disclosure, and which does not destroy the activity of the antibody. The excipient, carrier or adjuvant should be nontoxic when administered with an antibody in doses sufficient to deliver a therapeutic effect.

As used herein, the term “ameliorating” means a lessening or improvement of one or more symptoms as compared to not administering an antibody molecule of the invention. “Ameliorating” also includes shortening or reduction in duration of a symptom.

As used herein, the terms “prevent”, “preventing” and “prevention” refer to the prevention of the recurrence or onset of one or more symptoms of a disorder in a subject as result of the administration of a prophylactic or therapeutic agent.

Potency is a measure of the activity of a therapeutic agent expressed in terms of the amount required to produce an effect of given intensity. A highly potent agent evokes a greater response at low concentrations compared to an agent of lower potency that evokes a smaller response at low concentrations. Potency is a function of affinity and efficacy. Efficacy refers to the ability of therapeutic agent to produce a biological response upon binding to a target ligand and the quantitative magnitude of this response.

### BIOLOGICAL DEPOSITS

Representative materials of the present invention were deposited in the American Type Culture Collection, 10801 University Boulevard, Manassas, VA 20110-2209, USA, on 13¹′ September 2024. See, Table 1, infra.

TABLE 1

| | | ATCC |
|---|---|---|
| Antibody | Description | Accession No. |
| GBT-IgA-1602 HC1 | Anti MIGIS-α heavy chain 1 | PTA-127796 |
| GBT-IgA-1602 LC1 | Anti MIGIS-α light chain 1 | PTA-127794 |
| GBT-IgA-1602 HC2 | Anti CD3 heavy chain 2 | PTA-127797 |
| GBT-IgA-1602 LC2 | Anti CD3 light chain 2 | PTA-127795 |

The deposits were made under the provisions of the Budapest Treaty on the International Recognition of the Deposit of Microorganisms for the Purpose of Patent Procedure and Regulations thereunder (Budapest Treaty). This assures maintenance of a viable culture of the deposit for 30 years from the date of deposit. The deposit will be made available by ATCC under the terms of the Budapest Treaty, and subject to an agreement between Pfizer Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 U.S.C. Section 122 and the Commissioner's rules pursuant thereto (including 37 C.F.R. Section 1.14 with particular reference to 886 OG 638).

The assignee of the present application has agreed that if a culture of the materials on deposit should die or be lost or destroyed when cultivated under suitable conditions; the materials will be promptly replaced on notification with another of the same. Availability of the deposited material is not to be construed as a license to practice the invention in contravention of the rights granted under the authority of any government in accordance with its patent laws.

### Materials and Methods

Various techniques for the production of antibodies have been described which include the traditional hybridoma method for making monoclonal antibodies, recombinant techniques for making antibodies (including chimeric antibodies, e.g., humanized antibodies), antibody production in transgenic animals and the recently described phage display technology for preparing “fully human” antibodies.

Provided herein are methods of making any of the antibodies provided herein. The antibodies of this invention can be made by procedures known in the art. The polypeptides can be produced by proteolytic or other degradation of the antibodies, by recombinant methods (i.e., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available. For example, an antibody could be produced by an automated polypeptide synthesizer employing the solid phase method. See also, U.S. Pat. Nos. 5,807,715; 4,816,567; and 6,331,415.

Any suitable method for preparing multispecific antibodies may be used to prepare multispecific antibodies provided herein (e.g. depending on the choice of antibody features and components).

According to one approach to making multispecific antibodies, antibody variable domains with the desired binding specificities are fused to immunoglobulin constant region sequences. The fusion preferably is with an immunoglobulin heavy chain constant region, comprising at least part of the hinge, CH2 and CH3 regions. In some embodiments, the first heavy chain constant region (CH1), containing the site for light chain binding can be present in at least one of the fusions. In some embodiments, polynucleotides encoding the immunoglobulin heavy chain fusions and, if desired, the immunoglobulin light chain, may be inserted into separate expression vectors, and may be cotransfected into a suitable host organism. In other embodiments the coding sequences for two or all three polypeptide chains may be inserted into one expression vector when the expression of at least two polypeptide chains in equal ratios results in high yields or when the ratios are of no particular significance.

In one approach, the multispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the multispecific molecule, facilitates the separation of the desired multispecific compound from unwanted immunoglobulin chain combinations. This approach is described in PCT Publication No. WO 94/04690.

In another approach, the multispecific antibodies are composed of amino acid modification in the first hinge region in one arm, and the substituted amino acid in the first hinge region has an opposite charge to the corresponding amino acid in the second hinge region in another arm. This approach is described in International Patent Application No. PCT/US2011/036419 (WO2011/143545).

In another approach, the formation of a desired heteromultimeric or heterodimeric protein (e.g., bispecific antibody) is enhanced by altering or engineering an interface between a first and a second Fc chain. In this approach, the multispecific antibodies may be composed of a CH3 region, wherein the CH3 region comprises a first CH3 polypeptide and a second CH3 polypeptide which interact together to form a CH3 interface, wherein one or more amino acids within the CH3 interface destabilize homodimer formation and are not electrostatically unfavorable to homodimer formation. This approach is described in International Patent Application No. PCT/US2011/036419 (WO2011/143545). In some embodiments, one Fc chain of a bispecific antibody can comprise amino acid modifications at positions 223 and 228 (e.g., (C223E or C223R), and (P228E or P228R)) in the hinge region and at position 409 (e.g., K409R (EU numbering scheme)) in the CH3 region of human IgG2, and the other Fc chain of the bispecific antibody can comprise amino acid modifications at positions 223, 225 and 228 (e.g., (C223E or C223R), (E225R), and (P228E or P228R)) in the hinge region and at position 368 (e.g., L368E (EU numbering scheme)) in the CH3 region of human IgG2. In other embodiments, one Fc chain of a bispecific antibody can comprise amino acid modifications at positions 223 and 228 (e.g., (C223E or C223R) and (P228E or P228R)) in the hinge region and at position 368 (e.g., L368E (EU numbering scheme)) in the CH3 region of human IgG2, and the other Fc chain of the bispecific antibody can comprise amino acid modifications at positions 223, 225 and 228 (e.g., (C223E or C223R), (E225R), and (P228E or P228R)) in the hinge region and at position 409 (e.g., K409R (EU numbering scheme)) in the CH3 region of human IgG2. In some embodiments, a bispecific antibody can comprise amino acid modifications at positions 221 and 228 (e.g., (D221R or D221E) and (P228R or P228E)) in the hinge region and at position 409 or 368 (e.g., K409R or L368E (EU numbering scheme)) in the CH3 region of human IgG1. In some embodiments, a bispecific antibody can comprise amino acid modifications at positions 228 (e.g., (P228E or P228R)) in the hinge region and at position 409 or 368 (e.g., R409 or L368E (EU numbering scheme)) in the CH3 region of human IgG4.

In some embodiments, a multispecific antibody may have knob-in-hole mutations in the Fc chains. For example, in some embodiments, in a bispecific antibody having knob-in-hole mutations, the first Fc chain of the antibody Fc domain has one or more mutations to form a “knob”, and the second Fc chain of the antibody Fc domain has one or more mutations to form a “hole” (or vice-versa). Exemplary knob-in-hole engineering of antibodies is described in U.S. Pat. No. 5,731,168, PCT Publication No. WO2009089004, U.S. Publication No. 20090182127, Marvin and Zhu, Acta Pharmacologica Sincia (2005) 26(6):649-658 and Kontermann (2005) Acta Pharacol. Sin., 26:1-9.

A “knob” refers to at least one amino acid side chain which projects from the interface of a first polypeptide (e.g. first Fc chain) and is therefore positionable in a compensatory hole in an adjacent second polypeptide (e.g. second Fc chain) so as to stabilize a heterodimer, and thereby favor heterodimer formation over homodimer formation. The knob may exist in the original interface or may be introduced synthetically (e.g., by altering a nucleic acid encoding the interface). Normally, nucleic acid encoding the interface of the first polypeptide is altered to encode the knob. To achieve this, the nucleic acid encoding at least one original amino acid residue in the first polypeptide is replaced with nucleic acid encoding at least one “import” amino acid residue which has a larger side chain volume than the original amino acid residue. Certain import residues for the formation of a knob are generally naturally occurring amino acid residues and are preferably selected from arginine (R), phenylalanine (F), tyrosine (Y) and tryptophan (W).

A “hole” refers to at least one amino acid side chain which is recessed from the interface of a second polypeptide (e.g. second Fc chain) and therefore accommodates a corresponding knob in an adjacent first polypeptide (e.g. first Fc chain). The hole may exist in the original interface or may be introduced synthetically (e.g., by altering a nucleic acid encoding the interface). Normally, nucleic acid encoding the interface of the second polypeptide is altered to encode the hole. To achieve this, the nucleic acid encoding at least one original amino acid residue of the second polypeptide is replaced with DNA encoding at least one “import” amino acid residue which has a smaller side chain volume than the original amino acid residue. Certain import residues for the formation of a hole are usually naturally occurring amino acid residues and are preferably selected from alanine (A), serine (S), threonine (T) and valine (V).

The term “interface,” as used herein typically refers to any amino acid residue present in the domain that can be involved in first polypeptide and second polypeptide contacts. An “original amino acid” residue is one which is replaced by an “import amino acid” residue which can have a smaller or larger side chain volume than the original residue. The import amino acid residue can be a naturally occurring or non-naturally occurring amino acid residue, but preferably is the former. “Naturally occurring” amino acid residues are those residues encoded by the genetic code. By “non-naturally occurring” amino acid residue is meant a residue which is not encoded by the genetic code, but which is able to covalently bind adjacent amino acid residue(s) in the polypeptide chain. Examples of non-naturally occurring amino acid residues are norleucine, ornithine, norvaline, homoserine and other amino acid residue analogues such as those described in Ellman et al., Meth. Enzym. 202:301-336 (1991).

Once a nucleic acid sequence encoding molecules of the invention (i.e., binding domains) has been obtained, the vector for the production of the molecules may be produced by recombinant DNA technology using techniques well known in the art.

The polynucleotides encoding the antibody (binding domains of the present invention may include an expression control polynucleotide sequence operably linked to the antibody coding sequences, including naturally-associated or heterologous promoter regions known in the art. The expression control sequences may be eukaryotic promoter systems in vectors capable of transforming or transfecting eukaryotic host cells, but control sequences for prokaryotic hosts may also be used. Once the vector has been incorporated into the appropriate host cell line, the host cell is propagated under conditions suitable for expressing the nucleotide sequences, and, as desired, for the collection and purification of the antibodies. Eukaryotic cell lines include the CHO cell lines, various COS cell lines, HeLa cells, myeloma cell lines, transformed B-cells, or human embryonic kidney cell lines.

In one embodiment, the DNA encoding the antibodies of the invention is isolated and sequenced using conventional procedures (e.g., by using oligonucleotide probes that are capable of binding specifically to genes encoding the heavy and light chains of antibodies). Once isolated, the DNA may be placed into expression vectors, which are then transfected into host cells such as simian COS cells, CHO cells, or myeloma cells that do not otherwise produce immunoglobulin protein, to obtain the synthesis of monoclonal antibodies in the recombinant host cells. The DNA also may be modified, for example, to improve one or more properties of the corresponding antibody (e.g. binding affinity, immunogenicity, etc.).

In one aspect, the invention provides a method of making any of the polynucleotides described herein. For example, the polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art (e.g., Sambrook et al., 1989).

Alternatively, PCR allows reproduction of DNA sequences. PCR technology is well known in the art and is described in U.S. Pat. Nos. 4,683,195, 4,800,159, 4,754,065 and 4,683,202, as well as PCR: The Polymerase Chain Reaction, Mullis et al. eds., Birkauswer Press, Boston, 1994.

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, supra, for example.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

Any host cells capable of over-expressing heterologous DNAs can be used for the purpose expressing genes encoding the antibody, polypeptide or protein of interest. Non-limiting examples of mammalian host cells include but not limited to COS, HeLa, and CHO cells. See also PCT Publication No. WO87/04462. Suitable non-mammalian host cells include prokaryotes (such as E. coli or B. subtillis) and yeast (such as S. cerevisae, S. pombe; or K. lactis). A cell overexpressing the antibody or protein of interest can be identified by known screening methods.

In addition to the choice of host cells, factors that affect glycosylation during recombinant production of antibodies include growth mode, media formulation, culture density, oxygenation, pH, purification schemes and the like. Various methods have been proposed to alter the glycosylation pattern achieved in a particular host organism including introducing or overexpressing certain enzymes involved in oligosaccharide production (U.S. Pat. Nos. 5,047,335; 5,510,261 and 5,278,299). Glycosylation, or certain types of glycosylation, can be enzymatically removed from the glycoprotein, for example, using endoglycosidase H (Endo H), N-glycosidase F, endoglycosidase F1, endoglycosidase F2, endoglycosidase F3. In addition, the recombinant host cell can be genetically engineered to be defective in processing certain types of polysaccharides. These and similar techniques are well known in the art.

Other methods of modification include using coupling techniques known in the art, including, but not limited to, enzymatic means, oxidative substitution and chelation. Modifications can be used, for example, for attachment of labels for immunoassay. Modified polypeptides are made using established procedures in the art and can be screened using standard assays known in the art, some of which are described below and in the Examples.

In some embodiments of the invention, the antibody comprises a modified constant region, such as a constant region that has increased affinity to a human Fc gamma receptor, is immunologically inert or partially inert, e.g., does not trigger complement mediated lysis, does not stimulate antibody-dependent cell mediated cytotoxicity (ADCC), or does not activate macrophages; or has reduced activities (compared to the unmodified antibody) in any one or more of the following: triggering complement mediated lysis, stimulating antibody-dependent cell mediated cytotoxicity (ADCC), or activating microglia. Different modifications of the constant region may be used to achieve optimal level or combination of effector functions. See, for example, Morgan et al., Immunology 86:319-324, 1995; Lund et al., J. Immunology 157:4963-9 157:4963-4969, 1996; Idusogie et al., J. Immunology 164:4178-4184, 2000; Tao et al., J. Immunology 143: 2595-2601, 1989; and Jefferis et al., Immunological Reviews 163:59-76, 1998. In some embodiments, the constant region is modified as described in Eur. J. Immunol., 29:2613-2624, 1999; PCT Application No. PCT/GB99/01441; and/or UK Application No. 9809951.8. In still other embodiments, the constant region is aglycosylated for N-linked glycosylation. In some embodiments, the constant region is aglycosylated for N-linked glycosylation by mutating the glycosylated amino acid residue or flanking residues that are part of the N-glycosylation recognition sequence in the constant region. For example, N-glycosylation site N297 may be mutated to A, Q, K, or H. See, Tao et al., J. Immunology 143: 2595-2601, 1989; and Jefferis et al., Immunological Reviews 163:59-76,1998. In some embodiments, the constant region is aglycosylated for N-linked glycosylation. The constant region may be aglycosylated for N-linked glycosylation enzymatically (such as removing carbohydrate by enzyme PNGase), or by expression in a glycosylation deficient host cell.

Other antibody modifications include antibodies that have been modified as described in PCT Publication No. WO99/58572. These antibodies comprise, in addition to a binding domain directed at the target molecule, an effector domain having an amino acid sequence substantially homologous to all or part of a constant region of a human immunoglobulin heavy chain. These antibodies are capable of binding the target molecule without triggering significant complement dependent lysis, or cell-mediated destruction of the target. In some embodiments, the effector domain is capable of specifically binding either or both of the FcRn or the FcγRIIb. These are typically based on chimeric domains derived from two or more human immunoglobulin heavy chain CH2 domains. Antibodies modified in this manner are particularly suitable for use in chronic antibody therapy, to avoid inflammatory and other adverse reactions to conventional antibody therapy.

In some embodiments, the Fc chain of an antibody provided herein may be modified to ablate effector function. For example, the Fc chain of human IgG1 may be modified to introduce mutations L234A, L235A and G237A using standard primer-directed PCR mutagenesis to oblate effector function due to binding to FcγRIII, providing for an effector function null phenotype (Canfield et al., J. Exp. Med (1991) 173: 1483-1491; Shields et al., J. Biol. Chem. (2001) 276:6591-604).

In some embodiments, a multispecific antibody provided herein may be engineered to comprise at least one cysteine residue that may interact with a counterpart cysteine residue on another polypeptide chain of the invention to form an inter-chain disulfide bond. The inter-chain disulfide bonds may serve to stabilize the multispecific antibody, improving expression and recovery in recombinant systems, resulting in a stable and consistent formulation, as well as, improving the stability of the isolated and/or purified product in vivo. The cysteine residue or residues may be introduced as a single amino acid or as part of larger amino-acid sequence, e.g., hinge region, in any portion of the polypeptide chain. In a specific aspect, at least one cysteine residue is engineered to occur at the C-terminus of the polypeptide chain.

The foregoing description and following Examples detail certain specific embodiments of the disclosure and describes the best mode contemplated by the inventors. It will be appreciated, however, that no matter how detailed the foregoing may appear in text, the disclosure may be practiced in many ways and the disclosure should be construed in accordance with the appended claims and any equivalents thereof.

Although the disclosed teachings have been described with reference to various applications, methods, kits, and compositions, it will be appreciated that various changes and modifications can be made without departing from the teachings herein and the claimed disclosure below. The following examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

TABLE 2
SEQUENCE LISTING.

| SEQ | DESCRIPTION | SEQUENCE |
|---|---|---|
| 1 | MIGIS-α CDRH1 | GFEFSHYGMH |
| | GBT-IgA-1091 | |
| | GBT-IgA-1179 | |
| | GBT-IgA-1449 | |
| | GBT-IgA-1470 | |
| | GBT-IgA-1524 | |
| | GBT-IgA-1572 | |
| | GBT-IgA-1602 | |
| | GBT-IgA-1623 | |
| 2 | MIGIS-α CDRH1 | GFTFSHYGMH |
| | GBT-IgA-0860 | |
| | GBT-IgA-0738 | |
| 3 | MIGIS-α CDRH1 | GFTFSSYGMH |
| | GBT-IgA-0027 | |
| | (T18), | |
| | GBT-IgA-0032 | |
| | GBT-IgA-0033 | |
| | (H05) | |
| 4 | MIGIS-α CDRH2 | VISYDASHKY YADSVKG |
| | GBT-IgA-0738 | |
| | GBT-IgA-0752 | |
| | GBT-IgA-0860 | |
| | GBT-IgA-1090 | |
| | GBT-IgA-1091 | |
| | GBT-IgA-1164 | |
| | GBT-IgA-1165 | |
| | GBT-IgA-1179 | |
| | GBT-IgA-1445 | |
| | GBT-IgA-1446 | |
| | GBT-IgA-1449 | |
| | GBT-IgA-1470 | |
| | GBT-IgA-1524 | |
| | GBT-IgA-1572 | |
| | GBT-IgA-1602 | |
| | GBT-IgA-1623 | |
| 5 | MIGIS-α CDRH2 | VISYDGSNKYYADSVKG |
| | GBT-IgA-0027 | |
| | (T18) | |
| | GBT-IgA-0032 | |
| | GBT-IgA-0033 | |
| | (H05), | |
| 6 | MIGIS-α CDRH3 | LYGGNPEDAIDY |
| | GBT-IgA-0033 | |
| | (H05) | |
| 7 | MIGIS-α CDRH3 | LYGGNPKDTIYH |
| | GBT-IgA-0027 | |
| | GBT-IgA-0032 | |
| | GBT-IgA-0738 | |
| | GBT-IgA-0752 | |
| | GBT-IgA-0860 | |
| | GBT-IgA-1090 | |
| | GBT-IgA-1091 | |
| | GBT-IgA-1164 | |
| | GBT-IgA-1165 | |
| | GBT-IgA-1179 | |
| | GBT-IgA-1445 | |
| | GBT-IgA-1446 | |
| | GBT-IgA-1449 | |
| | GBT-IgA-1470 | |
| | GBT-IgA-1524 | |
| | GBT-IgA-1572 | |
| | GBT-IgA-1602 | |
| | GBT-IgA-1623 | |
| 8 | MIGIS-α CDRL1 | RASQSVSSNLA |
| | GBT-IgA-0027 | |
| | GBT-IgA-0032 | |
| | GBT-IgA-0033 | |
| | GBT-IgA-0738 | |
| | GBT-IgA-0752 | |
| | GBT-IgA-0860 | |
| | GBT-IgA-1090 | |
| | GBT-IgA-1091 | |
| | GBT-IgA-1164 | |
| | GBT-IgA-1165 | |
| | GBT-IgA-1179 | |
| | GBT-IgA-1445 | |
| | GBT-IgA-1446 | |
| | GBT-IgA-1449 | |
| | GBT-IgA-1602 | |
| 9 | MIGIS-α CDRL1 | RASQSVSSDLA |
| | GBT-IgA-1470 | |
| | GBT-IgA-1524 | |
| | GBT-IgA-1572 | |
| | GBT-IgA-1623 | |
| 10 | MIGIS-α CDRL2 | GASTRAT |
| | GBT-IgA-0027 | |
| | GBT-IgA-0032 | |
| | GBT-IgA-0033 | |
| | GBT-IgA-0071 | |
| | GBT-IgA-0658 | |
| | GBT-IgA-0661 | |
| | GBT-IgA-0738 | |
| | GBT-IgA-0752 | |
| | GBT-IgA-0860 | |
| | GBT-IgA-1090 | |
| | GBT-IgA-1091 | |
| | GBT-IgA-1164 | |
| | GBT-IgA-1165 | |
| | GBT-IgA-1179 | |
| | GBT-IgA-1445 | |
| | GBT-IgA-1446 | |
| | GBT-IgA-1449 | |
| | GBT-IgA-1470 | |
| | GBT-IgA-1524 | |
| | GBT-IgA-1572 | |
| | GBT-IgA-1602 | |
| | GBT-IgA-1623 | |
| 11 | MIGIS-α CDRL3 | QQYNNWPPFT |
| | GBT-IgA-0027 | |
| | GBT-IgA-0033 | |
| | GBT-IgA-0071 | |
| | GBT-IgA-0658 | |
| | GBT-IgA-0661 | |
| | GBT-IgA-0738 | |
| | GBT-IgA-0752 | |
| | GBT-IgA-0860 | |
| | GBT-IgA-1090 | |
| | GBT-IgA-1091 | |
| | GBT-IgA-1164 | |
| | GBT-IgA-1165 | |
| | GBT-IgA-1179 | |
| | GBT-IgA-1445 | |
| | GBT-IgA-1446 | |
| | GBT-IgA-1449 | |
| | GBT-IgA-1602 | |
| 12 | MIGIS-α CDRL3 | QTNHHWPPFT |
| | GBT-IgA-1470 | |
| | GBT-IgA-1524 | |
| | GBT-IgA-1572 | |
| | GBT-IgA-1623 | |
| 13 | MIGIS-α CDRL3 | EQYKNWPPFT |
| | GBT-IgA-0032 | |
| 14 | MIGIS-α VH | EVQLVESGGGLVQPGGSLRLSCAASGFEFSHYGMHWVRQAPGKGLEWVAVISYD |
| | GBT-IgA-1091 | ASHKYYADSVKGRFTISRDNAKNSLYLQMNSLRAEDTAVYYCAKLYGGNPKDTI |
| | GBT-IgA-1179 | YHWGQGTLVTVSS |
| | GBT-IgA-1449 | |
| | GBT-IgA-1470 | |
| | GBT-IgA-1524 | |
| | GBT-IgA-1572 | |
| | GBT-IgA-1602 | |
| | GBT-IgA-1623 | |
| 15 | MIGIS-α VH | EVQLVESGGG LVQPGGSLRL SCAASGFTFS HYGMHWVRQA PGKGLEWVAV |
| | GBT-IgA-0860 | ISYDASHKYY ADSVKGRFTI SRDNAKNSLY LQMNSLRAED TAVYYCAKLY |
| | GBT-IgA-1090 | GGNPKDTIYH WGQGTLVTVS S |
| | GBT-IgA-1165 | |
| | GBT-IgA-1446 | |
| 16 | Anti-MIGIS-α | GAAATTGTGTTGACGCAGTCTCCAGCCACCCTGTCTGTGTCTCCAGGGGAAAGA |
| | Light Chain | GCCACCCTCTCCTGCAGGGCCAGTCAGAGTGTTAGCAGCAACTTAGCCTGGTAC |
| | DNA sequence | CAGCAAAAACCTGGCCAGGCTCCCAGGCTCCTCATCTATGGTGCATCCACCAGG |
| | GBT-IgA-1449 | GCCACTGGTATCCCAGCCAGGTTCAGTGGCAGTGGGTCTGGGACAGAGTTCACT |
| | GBT-IgA-1602 | CTCACCATCAGCAGCCTGCAGTCTGAAGATTTTGCAGTTTATTACTGTCAGCAG |
| | | TATAATAACTGGCCTCCTTTCACTTTCGGCCCTGGGACCAAAGTGGATATCAAA |
| | | CGTACGGTGGCTGCACCATCTGTCTTCATCTTCCCGCCATCTGATGAGCAGTTG |
| | | AAATCTGGAACTGCCTCTGTTGTGTGCCTGCTGAATAACTTCTATCCCAGAGAG |
| | | GCCAAAGTACAGTGGAAGGTGGATAACGCCCTCCAATCGGGTAACTCCCAGGAG |
| | | AGTGTCACAGAGCAGGACAGCAAGGACAGCACCTACAGCCTCAGCAGCACCCTG |
| | | ACGCTGAGCAAAGCAGACTACGAGAAACACAAAGTCTACGCCTGCGAAGTCACC |
| | | CATCAGGGCCTGAGCTCGCCCGTCACAAAGAGCTTCAACAGGGGAGAGTGT |
| 17 | Anti-MIGIS-α | GAGGTGCAGCTGGTGGAGTCTGGGGGAGGCTTGGTCCAGCCTGGGGGGTCCCTG |
| | Heavy Chain | AGACTCTCCTGTGCAGCCTCTGGATTCGAGTTTAGTCATTATGGCATGCACTGG |
| | DNA sequence | GTCCGCCAGGCTCCAGGGAAGGGGCTGGAGTGGGTGGCCGTTATATCATATGAT |
| | GBT-IgA-1449 | GCAAGTCACAAATACTATGCAGACTCTGTGAAGGGCCGATTCACCATCTCCAGA |
| | GBT-IgA-1602 | GACAACGCCAAGAACTCACTGTATCTGCAAATGAACAGCCTGAGAGCCGAGGAC |
| | | ACGGCTGTGTATTACTGTGCGAAACTCTACGGTGGTAATCCTAAGGATACTATT |
| | | TACCACTGGGGCCAGGGCACCCTGGTCACCGTCTCCTCAGCGTCGACCAAGGGC |
| | | CCATCGGTCTTCCCCCTGGCACCCTCCTCCAAGAGCACCTCTGGGGGCACAGCG |
| | | GCCCTGGGCTGCCTGGTCAAGGACTACTTCCCCGAACCGGTGACGGTGTCGTGG |
| | | AACTCAGGCGCCCTGACCAGCGGCGTGCACACCTTCCCGGCTGTCCTACAGTCC |
| | | TCAGGACTCTACTCCCTCAGCAGCGTGGTGACCGTGCCCTCCAGCAGCTTGGGC |
| | | ACCCAGACCTACATCTGCAACGTGAATCACAAGCCCAGCAACACCAAGGTGGAC |
| | | AAGAAAGTTGAGCCCAAATCTTGTCGGAAAACTCACACATGCCCACCGTGCCCA |
| | | GCACCTGAAGCCGCTGGGGCACCGTCAGTCTTCCTCTTCCCCCCAAAACCCAAG |
| | | GACACCCTCATGATCTCCCGGACCCCTGAGGTCACATGCGTGGTGGTGGACGTG |
| | | AGCCACGAAGACCCTGAGGTCAAGTTCAACTGGTACGTGGACGGCGTGGAGGTG |
| | | CATAATGCCAAGACAAAGCCGCGGGAGGAGCAGTACAACAGCACGTACCGTGTG |
| | | GTCAGCGTCCTCACCGTCCTGCACCAGGACTGGCTGAATGGCAAGGAGTACAAG |
| | | TGCAAGGTCTCCAACAAAGCCCTCCCAGCCCCCATCGAGAAAACCATCTCCAAA |
| | | GCCAAAGGGCAGCCCCGAGAACCACAGGTGTACACCCTGCCCCCATCCCGGGAG |
| | | GAGATGACCAAGAACCAGGTCAGCCTGACCTGCCTGGTCAAAGGCTTCTATCCC |
| | | AGCGACATCGCCGTGGAGTGGGAGAGCAATGGGCAGCCGGAGAACAACTACAAG |
| | | ACCACGCCTCCCGTGCTGGACTCCGACGGCTCCTTCTTCCTCTATAGCAGGCTC |
| | | ACCGTGGACAAGAGCAGGTGGCAGCAGGGGAACGTCTTCTCATGCTCCGTGATG |
| | | CATGAGGCTCTGCACAACCACTACACGCAGAAGAGCCTCTCCCTGTCCCCGGGA |
| | | AAA |
| 18 | MIGIS-α VH | QVQLVESGGG VVQPGRSLRL SCAASGFTFS HYGMHWVRQA PGKGLEWVAV |
| | GBT-IgA-0738 | ISYDASHKYY ADSVKGRFTI SRDNSKNTLY LQMNSLRAED TAVYYCAKLY |
| | | GGNPKDTIYH WGRGTLVTVS S |
| 19 | MIGIS-α VH | QVQLVESGGGVVQPGRSLRLSCAASGFTFSSYGMHWVRQAPGKGLEWVAVISYD |
| | GBT-IgA-0027 | GSNKYYADSVKGRFTISRDNSKNTLYLQMNSLRAEDTAVYYCAKLYGGNPKDTI |
| | GBT-IgA-0032 | YHWGRGTLVTVSS |
| 20 | MIGIS-α VH | QVQLVESGGGVVQPGRSLRLSCAASGFTFSSYGMHWVRQAPGKGLEWVAVISYD |
| | GBT-IgA-0033 | GSNKYYADSVKGRFTISRDNSKNTLYLQMNSLRAEDTAVYYCAKLYGGNPEDAI |
| | | DYWGRGTLVTVSS |
| 21 | MIGIS-α VL | EIVLTQSPATLSVSPGERATLSCRASQSVSSNLAWYQQKPGQAPRLLIYGASTR |
| | GBT-IgA-0027 | ATGIPARFSGSGSGTEFTLTISSLQSEDFAVYYCQQYNNWPPFTFGPGTKVDIK |
| | GBT-IgA-0033 | |
| | GBT-IgA-0071 | |
| | GBT-IgA-0738 | |
| | GBT-IgA-0752 | |
| | GBT-IgA-0836 | |
| | GBT-IgA-0860 | |
| | GBT-IgA-1090 | |
| | GBT-IgA-1091 | |
| | GBT-IgA-1164 | |
| | GBT-IgA-1165 | |
| | GBT-IgA-1179 | |
| | GBT-IgA-1445 | |
| | GBT-IgA-1446 | |
| | GBT-IgA-1449 | |
| | GBT-IgA-1602 | |
| 22 | MIGIS-α CDRH1 | GFTFSSYAMS |
| | GBT-IgA-0020 | |
| | (15E7) | |
| | GBT-IgA-0034 | |
| | (ET10) | |
| 23 | MIGIS-α CDRH1 | GFTFSRYAMS |
| | GBT-IgA-0035 | |
| | (ET17) | |
| | GBT-IgA-0036 | |
| | (ET18) | |
| 24 | MIGIS-α CDRH2 | SITGSGGGTYYADPVKG |
| | GBT-IgA-0034 | |
| | (ET10) | |
| 25 | MIGIS-α CDRH2 | AITGSGSGTYYADPVKG |
| | GBT-IgA-0035 | |
| | (ET17) | |
| | GBT-IgA-0036 | |
| | (ET18) | |
| 26 | MIGIS-α CDRH3 | ERSGSSSDY |
| | GBT-IgA-0020 | |
| | (15E7) | |
| | GBT-IgA-0034 | |
| | (ET10) | |
| | GBT-IgA-0035 | |
| | (ET17) | |
| | GBT-IgA-0036 | |
| | (ET18) | |
| 27 | MIGIS-α CDRL1 | RASQTVSSYLA |
| | GBT-IgA-0020 | |
| | (15E7) | |
| 28 | MIGIS-α CDRL1 | RASQTVRSHLA |
| | GBT-IgA-0034 | |
| | (ET10) | |
| 29 | MIGIS-α CDRL1 | RASQTVGSHLA |
| | GBT-IgA-0035 | |
| | (ET17) | |
| 30 | MIGIS-α CDRL1 | RASQTVSSHLA |
| | GBT-IgA-0036 | |
| | (ET18) | |
| 31 | MIGIS-α CDRL2 | EASNRAT |
| | GBT-IgA-0020 | |
| | (15E7) | |
| | GBT-IgA-0034 | |
| | (ET10) | |
| | GBT-IgA-0035 | |
| | (ET17) | |
| | GBT-IgA-0036 | |
| | (ET18) | |
| 32 | MIGIS-α CDRL3 | QQRNNWPIT |
| | GBT-IgA-0020 | |
| | (15E7) | |
| | GBT-IgA-0034 | |
| | (ET10) | |
| | GBT-IgA-0035 | |
| | (ET17) | |
| | GBT-IgA-0036 | |
| | (ET18) | |
| 33 | MIGIS-α VL | EIVLTQSPATLSVSPGERATLSCRASQSVSSNLAWYQQKPGQAPRLLIYGASTR |
| | GBT-IgA-0032 | ATGIPARFSGSGSGTEFTLTISSLQSEDFAVYYCEQYKNWPPFTFGPGTKVDIK |
| 34 | MIGIS-α VL | EIVLTQSPATLSVSPGERATLSCRASQSVSSDLAWYQQKPGQAPRLLIYGASTR |
| | GBT-IgA-1470 | ATGIPARFSGSGSGTEFTLTISSLQSEDFAVYYCQTNHHWPPFTFGPGTKVDIK |
| | GBT-IgA-1524 | |
| | GBT-IgA-1572 | |
| | GBT-IgA-1623 | |
| 35 | MIGIS-α VH | EVQLLESGGGLVQPGGSLRLSCAASGFTFSSYAMSWVRQVPGKGLEWVSAITGS |
| | GBT-IgA-0020 | GSGTYYADPVKGRFTISRDNSKNTLYLQMNSLRAEDTAVYHCAKERSGSSSDYW |
| | (15E7) | GQGTLVTVSS |
| 36 | MIGIS-α VL | EIVLTQSPATLSLSPGERATLSCRASQTVSSYLAWYQQKPGQAPRLLIYEASNR |
| | GBT-IgA-0020 | ATGIPPRFSGSGSGTDFTLTISSLEPEDFAVYYCQQRNNWPITFGQGTRLESK |
| | (15E7) | |
| 37 | MIGIS-α VH | EVQLLESGGGLVQPGGSLRLSCAASGFTFSSYAMSWVRQVPGKGLEWVSSITGS |
| | GBT-IgA-0034 | GGGTYYADPVKGRFTISRDNSKNTLYLQMNSLRAEDTAVYHCAKERSGSSSDYW |
| | (ET10) | GQGTLVTVSS |
| 38 | MIGIS-α VL | EIVLTQSPATLSLSPGERATLSCRASQTVRSHLAWYQQKPGQAPRLLIYEASNR |
| | GBT-IgA-0034 | ATGIPPRFSGSGSGTDFTLTISSLEPEDFAVYYCQQRNNWPITFGQGTRLESK |
| | (ET10) | |
| 39 | MIGIS-α VH | EVQLLESGGGLVQPGGSLRLSCAASGFTFSRYAMSWVRQVPGKGLEWVSAITGS |
| | GBT-IgA-0035 | GSGTYYADPVKGRFTISRDNSKNTLYLQMNSLRAEDTAVYHCAKERSGSSSDYW |
| | (ET17) | GQGTLVTVSS |
| 40 | MIGIS-α VL | EIVLTQSPATLSLSPGERATLSCRASQTVGSHLAWYQQKPGQAPRLLIYEASNR |
| | GBT-IgA-0035 | ATGIPPRFSGSGSGTDFTLTISSLEPEDFAVYYCQQRNNWPITFGQGTRLESK |
| | (ET17) | |
| 41 | MIGIS-α VH | EVQLLESGGGLVQPGGSLRLSCAASGFTFSRYAMSWVRQVPGKGLEWVSAITGS |
| | GBT-IgA-0036 | GSGTYYADPVKGRFTISRDNSKNTLYLQMNSLRAEDTAVYHCAKERSGSSSDYW |
| | (ET18) | GQGTLVTVSS |
| 42 | MIGIS-α VL | EIVLTQSPATLSLSPGERATLSCRASQTVSSHLAWYQQKPGQAPRLLIYEASNR |
| | GBT-IgA-0036 | ATGIPPRFSGSGSGTDFTLTISSLEPEDFAVYYCQQRNNWPITFGQGTRLESK |
| | (ET18) | |
| 43 | Constant | -ASTKGPSVF PLAPSSKSTS GGTAALGCLV KDYFPEPVTV SWNSGALTSG |
| | heavy EFN RR | VHTFPAVLQS SGLYSLSSVV TVPSSSLGTQ TYICNVNHKP SNTKVDKKVE |
| | Effector | PKSCRKTHTC PPCPAPEAAG APSVELFPPK PKDTLMISRT PEVTCVVVDV |
| | function null | SHEDPEVKFN WYVDGVEVHN AKTKPREEQY NSTYRVVSVL TVLHQDWLNG |
| | mutations: | KEYKCKVSNK ALPAPIEKTI SKAKGQPREP QVYTLPPSRE EMTKNQVSLT |
| | L234A, L235A | CLVKGFYPSD IAVEWESNGQ PENNYKTTPP VLDSDGSFFL YSRLTVDKSR |
| | and G237A | WQQGNVFSCS VMHEALHNHY TQKSLSLSPG K |
| | (EU) | |
| | Heterodimerizing | |
| | mutations: | |
| | D221R, K409R | |
| | (EU) | |
| | GBT-IgA-0658 | |
| | GBT-IgA-0661 | |
| | GBT-IgA-1164 | |
| | GBT-IgA-1165 | |
| | GBT-IgA-1445 | |
| | GBT-IgA-1446 | |
| | GBT-IgA-1449 | |
| | GBT-IgA-1470 | |
| | GBT-IgA-1602 | |
| | GBT-IgA-1623 | |
| 44 | Constant | _ASTKGPSVF PLAPSSKSTS GGTAALGCLV KDYFPEPVTV SWNSGALTSG |
| | Heavy Chain | VHTFPAVLQS SGLYSLSSVV TVPSSSLGTQ TYICNVNHKP SNTKVDKKVE |
| | one armed | PKSCDKTHTC PPCPAPEAAG APSVFLFPPK PKDTLMISRT PEVTCVVVDV |
| | antibody | SHEDPEVKFN WYVDGVEVHN AKTKPREEQY NSTYRVVSVL TVLHQDWLNG |
| | GBT-IgA-0070 | KEYKCKVSNK ALPAPIEKTI SKAKGQPREP QVCTLPPSRE EMTKNQVSLW |
| | GBT-IgA-0071 | CLVKGFYPSD IAVEWESNGQ PENNYKTTPP VLDSDGSFFL YSKLTVDKSR |
| | GBT-IgA-0752 | WQQGNVFSCS VMHEALHNHY TQKSLSLSPG K |
| | GBT-IgA-1090 | |
| | GBT-IgA-1091 | |
| | GBT-IgA-1572 | |
| 45 | Heavy Chain | EPKSSDKTHT CPPCPAPEAA GAPSVFLFPP KPKDTLMISR TPEVTCVVVD |
| | Truncated | VSHEDPEVKF NWYVDGVEVH NAKTKPREEQ YNSTYRVVSV LTVLHQDWLN |
| | one armed | GKEYKCKVSN KALPAPIEKT ISKAKGQPRE PQVYTLPPCR EEMTKNQVSL |
| | antibody | SCAVKGFYPS DIAVEWESNG QPENNYKTTP PVLDSDGSFF LVSKLTVDKS |
| | GBT-IgA-0070 | RWQQGNVFSC SVMHEALHNR YTQKSLSLSP GK |
| | GBT-IgA-0071 | |
| | GBT-IgA-0752 | |
| | GBT-IgA-1090 | |
| | GBT-IgA-1091 | |
| | GBT-IgA-1572 | |
| 46 | Constant | _ASTKGPSVF PLAPSSKSTS GGTAALGCLV KDYFPEPVTV SWNSGALTSG |
| | heavy WT | VHTFPAVLQS SGLYSLSSVV TVPSSSLGTQ TYICNVNHKP SNTKVDKKVE |
| | huIgG1 | PKSCDKTHTC PPCPAPELLG GPSVFLFPPK PKDTLMISRT PEVTCVVVDV |
| | WT huIgG1 | SHEDPEVKFN WYVDGVEVHN AKTKPREEQY NSTYRVVSVL TVLHQDWLNG |
| | and | KEYKCKVSNK ALPAPIEKTI SKAKGQPREP QVYTLPPSRE EMTKNQVSLT |
| | afucosylated | CLVKGFYPSD IAVEWESNGQ PENNYKTTPP VLDSDGSFFL YSKLTVDKSR |
| | huIgG1 | WQQGNVFSCS VMHEALHNHY TQKSLSLSPG |
| | GBT-IgA-0027 | |
| | GBT-IgA-0032 | |
| | GBT-IgA-0033 | |
| | GBT-IgA-0860 | |
| | GBT-IgA-0862 | |
| | GBT-IgA-1179 | |
| | GBT-IgA-1524 | |
| 47 | Human kappa | _RTVAAPSVE IFPPSDEQLK SGTASVVCLL NNFYPREAKV QWKVDNALQS |
| | Constant | GNSQESVTEQ DSKDSTYSLS STLTLSKADY EKHKVYACEV THQGLSSPVT |
| | GBT-IgA-0020 | KSFNRGEC |
| | GBT-IgA-0027 | |
| | GBT-IgA-0033 | |
| | GBT-IgA-0034 | |
| | GBT-IgA-0035 | |
| | GBT-IgA-0036 | |
| | GBT-IgA-0070 | |
| | GBT-IgA-0071 | |
| | GBT-IgA-0658 | |
| | GBT-IgA-0661 | |
| | GBT-IgA-0752 | |
| | GBT-IgA-0860 | |
| | GBT-IgA-0836 | |
| | GBT-IgA-0862 | |
| | GBT-IgA-1090 | |
| | GBT-IgA-1091 | |
| | BMD-IgA-1156 | |
| | GBT-IgA-1164 | |
| | GBT-IgA-1165 | |
| | GBT-IgA-1445 | |
| | GBT-IgA-1446 | |
| | GBT-IgA-1449 | |
| | GBT-IgA-1470 | |
| | GBT-IgA-1524 | |
| | GBT-IgA-1572 | |
| | GBT-IgA-1179 | |
| | GBT-IgA-1602 | |
| | GBT-IgA-1623 | |
| 48 | Anti-MIGIS-α | EVQLVESGGG LVQPGGSLRL SCAASGFEFS HYGMHWVRQA PGKGLEWVAV |
| | Heavy Chain | ISYDASHKYY ADSVKGRFTI SRDNAKNSLY LQMNSLRAED TAVYYCAKLY |
| | EFN RR | GGNPKDTIYH WGQGTLVTVS SASTKGPSVF PLAPSSKSTS GGTAALGCLV |
| | GBT-IgA-1449 | KDYFPEPVTV SWNSGALTSG VHTFPAVLQS SGLYSLSSVV TVPSSSLGTQ |
| | GBT-IgA-1602 | TYICNVNHKP SNTKVDKKVE PKSCRKTHTC PPCPAPEAAG APSVFLFPPK |
| | | PKDTLMISRT PEVTCVVVDV SHEDPEVKFN WYVDGVEVHN AKTKPREEQY |
| | | NSTYRVVSVL TVLHQDWLNG KEYKCKVSNK ALPAPIEKTI SKAKGQPREP |
| | | QVYTLPPSRE EMTKNQVSLT CLVKGFYPSD IAVEWESNGQ PENNYKTTPP |
| | | VLDSDGSFFL YSRLTVDKSR WQQGNVFSCS VMHEALHNHY TQKSLSLSPG |
| | | K |
| 49 | Anti-CD3 | GACATCCAGATGACACAGAGCCCTAGCAGCCTGTCTGCCAGCGTGGGAGACAGA |
| | Light Chain | GTGACCATCACCTGTACCAGCAGCCAGAGCCTGTTCAACGTGCGGAGCCAGAAG |
| | DNA sequence | AACTACCTGGCCTGGTATCAGCAGAAGCCCGGCAAGGCTCCTAAGCTGCTGATC |
| | GBT-IgA-0661 | TACTGGGCCAGCACCAGAGAAAGCGGCGTGCCAAGCAGATTTTCTGGCAGCGGC |
| | BMD-IgA-1156 | TCTGGCACCGACTTCACCCTGACCATATCTAGCCTGCAGCCTGAGGACTTCGCC |
| | GBT-IgA-1164 | ACCTACTACTGCAAGCAGAGCTACGACCTGTTCACCTTCGGCGGAGGCACCAAG |
| | GBT-IgA-1165 | GTGGAAATCAAGCGTACGGTGGCTGCACCATCTGTCTTCATCTTCCCGCCATCT |
| | GBT-IgA-1602 | GATGAGCAGTTGAAATCTGGAACTGCCTCTGTTGTGTGCCTGCTGAATAACTTC |
| | GBT-IgA-1623 | TATCCCAGAGAGGCCAAAGTACAGTGGAAGGTGGATAACGCCCTCCAATCGGGT |
| | | AACTCCCAGGAGAGTGTCACAGAGCAGGACAGCAAGGACAGCACCTACAGCCTC |
| | | AGCAGCACCCTGACGCTGAGCAAAGCAGACTACGAGAAACACAAAGTCTACGCC |
| | | TGCGAAGTCACCCATCAGGGCCTGAGCTCGCCCGTCACAAAGAGCTTCAACAGG |
| | | GGAGAGTGT |
| 50 | Anti-MIGIS-α | EVQLVESGGG LVQPGGSLRL SCAASGFEFS HYGMHWVRQA PGKGLEWVAV |
| | Heavy Chain | ISYDASHKYY ADSVKGRFTI SRDNAKNSLY LQMNSLRAED TAVYYCAKLY |
| | EFN RR | GGNPKDTIYH WGQGTLVTVS SASTKGPSVF PLAPSSKSTS GGTAALGCLV |
| | GBT-IgA-1470 | KDYFPEPVTV SWNSGALTSG VHTFPAVLQS SGLYSLSSVV TVPSSSLGTQ |
| | GBT-IgA-1623 | TYICNVNHKP SNTKVDKKVE PKSCRKTHTC PPCPAPEAAG APSVFLFPPK |
| | | PKDTLMISRT PEVTCVVVDV SHEDPEVKFN WYVDGVEVHN AKTKPREEQY |
| | | NSTYRVVSVL TVLHQDWLNG KEYKCKVSNK ALPAPIEKTI SKAKGQPREP |
| | | QVYTLPPSRE EMTKNQVSLT CLVKGFYPSD IAVEWESNGQ PENNYKTTPP |
| | | VLDSDGSFFL YSRLTVDKSR WQQGNVFSCS VMHEALHNHY TQKSLSLSPG |
| | | K |
| 51 | Anti-MIGIS-α | EIVLTQSPAT LSVSPGERAT LSCRASQSVS SDLAWYQQKP GQAPRLLIYG |
| | Light Chain | ASTRATGIPA RFSGSGSGTE FTLTISSLQS EDFAVYYCQT NHHWPPFTFG |
| | GBT-IgA-1470 | PGTKVDIKRT VAAPSVFIFP PSDEQLKSGT ASVVCLLNNF YPREAKVQWK |
| | GBT-IgA-1623 | VDNALQSGNS QESVTEQDSK DSTYSLSSTL TLSKADYEKH KVYACEVTHQ |
| | | GLSSPVTKSF NRGEC |
| 52 | Anti-MIGIS-α | QVQLVESGGG VVQPGRSLRL SCAASGFTFS HYGMHWVRQA PGKGLEWVAV |
| | Heavy Chain | ISYDASHKYY ADSVKGRFTI SRDNSKNTLY LQMNSLRAED TAVYYCAKLY |
| | EFN RR | GGNPKDTIYH WGRGTLVTVS SASTKGPSVF PLAPSSKSTS GGTAALGCLV |
| | GBT-IgA-1164 | KDYFPEPVTV SWNSGALTSG VHTFPAVLQS SGLYSLSSVV TVPSSSLGTQ |
| | GBT-IgA-1445 | TYICNVNHKP SNTKVDKKVE PKSCRKTHTC PPCPAPEAAG APSVFLFPPK |
| | | PKDTLMISRT PEVTCVVVDV SHEDPEVKFN WYVDGVEVHN AKTKPREEQY |
| | | NSTYRVVSVL TVLHQDWLNG KEYKCKVSNK ALPAPIEKTI SKAKGQPREP |
| | | QVYTLPPSRE EMTKNQVSLT CLVKGFYPSD IAVEWESNGQ PENNYKTTPP |
| | | VLDSDGSFFL YSRLTVDKSR WQQGNVFSCS VMHEALHNHY TQKSLSLSPG |
| | | K |
| 53 | CID240 HC | MGWSCIILFLVATATGAHSQVQLVQSGAEVKKPGASVKVSCKASGYSFTYYGIS |
| | Human | WVRQAPGQGLEWMGWISTYNGNTNYARMLQGRVTMTTDTSTRTAYMELRSLRSD |
| | Membrane IgA | DTAVYYCARENYYGSGSYRGGMDVWGQGTTVTVSSASPTSPKVFPLSLCSTQPD |
| | Heavy Chain | GNVVIACLVQGFFPQEPLSVTWSESGQGVTARNFPPSQDASGDLYTTSSQLTLP |
| | | ATQCLAGKSVTCHVKHYTNPSQDVTVPCPVPSTPPTPSPSTPPTPSPSCCHPRL |
| | | SLHRPALEDLLLGSEANLTCTLTGLRDASGVTFTWTPSSGKSAVQGPPERDLCG |
| | | CYSVSSVLPGCAEPWNHGKTFTCTAAYPESKTPLTATLSKSGNTFRPEVHLLPP |
| | | PSEELALNELVTLTCLARGESPKDVLVRWLQGSQELPREKYLTWASRQEPSQGT |
| | | TTFAVTSILRVAAEDWKKGDTFSCMVGHEALPLAFTQKTIDRLADWQMPPPYVV |
| | | LDLPQETLEEETPGANLWPTTITELTLFLLSLFYSTALTVTSVRGPSGNREGPQ |
| | | Y |
| 54 | Anti-MIGIS-α | EVQLVESGGG LVQPGGSLRL SCAASGETFS HYGMHWVRQA PGKGLEWVAV |
| | Heavy Chain | ISYDASHKYY ADSVKGRFTI SRDNAKNSLY LQMNSLRAED TAVYYCAKLY |
| | EFN RR | GGNPKDTIYH WGQGTLVTVS SASTKGPSVF PLAPSSKSTS GGTAALGCLV |
| | GBT-IgA-1165 | KDYFPEPVTV SWNSGALTSG VHTFPAVLQS SGLYSLSSVV TVPSSSLGTQ |
| | GBT-IgA-1446 | TYICNVNHKP SNTKVDKKVE PKSCRKTHTC PPCPAPEAAG APSVELFPPK |
| | | PKDTLMISRT PEVTCVVVDV SHEDPEVKEN WYVDGVEVHN AKTKPREEQY |
| | | NSTYRVVSVL TVLHQDWLNG KEYKCKVSNK ALPAPIEKTI SKAKGQPREP |
| | | QVYTLPPSRE EMTKNQVSLT CLVKGFYPSD IAVEWESNGQ PENNYKTTPP |
| | | VLDSDGSFFL YSRLTVDKSR WQQGNVESCS VMHEALHNHY TQKSLSLSPG |
| | | K |
| 55 | Anti-CD3 | GAGGTGCAGCTGGTTGAATCTGGCGGAGGACTGGTTCAGCCTGGCGGATCTCTG |
| | Heavy Chain | AGACTGTCTTGTGCCGCCAGCGGCTTCACCTTCAGCGACTACTACATGACCTGG |
| | DNA sequence | GTCCGACAGGCCCCTGGCAAAGGACTTGAGTGGGTCGCCTTCATCCGGAACCAG |
| | GBT-IgA-0661 | GCCAGAGGCTACACCAGCGACCACAATCCTAGCGTGAAGGGCAGATTCACCATC |
| | BMD-IgA-1156 | AGCCGGGACAACGCCAAGAACAGCCTGTACCTGCAGATGAACTCCCTGAGAGCC |
| | GBT-IgA-1164 | GAGGACACCGCCGTGTACTACTGCGCCAGAGACAGACCCAGCTACTACGTGCTG |
| | GBT-IgA-1165 | GATTACTGGGGCCAGGGCACCACCGTGACAGTGTCTAGCGCGTCGACCAAGGGC |
| | GBT-IgA-1602 | CCATCGGTCTTCCCCCTGGCACCCTCCTCCAAGAGCACCTCTGGGGGCACAGCG |
| | GBT-IgA-1623 | GCCCTGGGCTGCCTGGTCAAGGACTACTTCCCCGAACCGGTGACGGTGTCGTGG |
| | | AACTCAGGCGCCCTGACCAGCGGCGTGCACACCTTCCCGGCTGTCCTACAGTCC |
| | | TCAGGACTCTACTCCCTCAGCAGCGTGGTGACCGTGCCCTCCAGCAGCTTGGGC |
| | | ACCCAGACCTACATCTGCAACGTGAATCACAAGCCCAGCAACACCAAGGTGGAC |
| | | AAGAAAGTTGAGCCCAAATCTTGTGAGAAAACTCACACATGCCCACCGTGCCCA |
| | | GCACCTGAAGCCGCTGGGGCACCGTCAGTCTTCCTCTTCCCCCCAAAACCCAAG |
| | | GACACCCTCATGATCTCCCGGACCCCTGAGGTCACATGCGTGGTGGTGGACGTG |
| | | AGCCACGAAGACCCTGAGGTCAAGTTCAACTGGTACGTGGACGGCGTGGAGGTG |
| | | CATAATGCCAAGACAAAGCCGCGGGAAGAGCAGTACAACAGCACGTACCGTGTG |
| | | GTCAGCGTCCTCACCGTCCTGCACCAGGACTGGCTGAATGGCAAGGAGTACAAG |
| | | TGCAAGGTCTCCAACAAAGCCCTCCCAGCCCCCATCGAGAAAACCATCTCCAAA |
| | | GCCAAAGGGCAGCCCCGAGAACCACAGGTGTACACCCTGCCCCCATCCCGGGAG |
| | | GAGATGACCAAGAACCAGGTCAGCCTGACCTGCGAGGTCAAAGGCTTCTATCCC |
| | | AGCGACATCGCCGTGGAGTGGGAGAGCAATGGGCAGCCGGAGAACAACTACAAG |
| | | ACCACGCCTCCCGTGCTGGACTCCGACGGCTCCTTCTTCCTCTATAGCAAGCTC |
| | | ACCGTGGACAAGAGCAGGTGGCAGCAGGGGAACGTCTTCTCATGCTCCGTGATG |
| | | CATGAGGCTCTGCACAACCACTACACGCAGAAGAGCCTCTCCCTGTCCCCGGGA |
| | | AAA |
| 56 | Anti-MIGIS-α | QVQLVESGGG VVQPGRSLRL SCAASGFTFS SYGMHWVRQA PGKGLEWVAV |
| | Heavy Chain | ISYDGSNKYY ADSVKGRFTI SRDNSKNTLY LQMNSLRAED TAVYYCAKLY |
| | EFN RR | GGNPKDTIYH WGRGTLVTVS SASTKGPSVF PLAPSSKSTS GGTAALGCLV |
| | GBT-IgA-0658 | KDYFPEPVTV SWNSGALTSG VHTFPAVLQS SGLYSLSSVV TVPSSSLGTQ |
| | GBT-IgA-0661 | TYICNVNHKP SNTKVDKKVE PKSCRKTHTC PPCPAPEAAG APSVFLFPPK |
| | | PKDTLMISRT PEVTCVVVDV SHEDPEVKFN WYVDGVEVHN AKTKPREEQY |
| | | NSTYRVVSVL TVLHQDWING KEYKCKVSNK ALPAPIEKTI SKAKGQPREP |
| | | QVYTLPPSRE EMTKNQVSLT CLVKGFYPSD IAVEWESNGQ PENNYKTTPP |
| | | VLDSDGSFFL YSRLTVDKSR WQQGNVFSCS VMHEALHNHY TQKSLSLSPG |
| 57 | Anti-MIGIS-α | EIVLTQSPAT LSVSPGERAT LSCRASQSVS SNLAWYQQKP GQAPRLLIYG |
| | Light Chain | ASTRATGIPA RFSGSGSGTE FTLTISSLQS EDFAVYYCQQ YNNWPPFTFG |
| | GBT-IgA-0027 | PGTKVDIKRT VAAPSVFIFP PSDEQLKSGT ASVVCLLNNF YPREAKVQWK |
| | GBT-IgA-0033 | VDNALQSGNS QESVTEQDSK DSTYSLSSTL TLSKADYEKH KVYACEVTHQ |
| | GBT-IgA-0070 | GLSSPVTKSF NRGEC |
| | GBT-IgA-0071 | |
| | GBT-IgA-0631 | |
| | GBT-IgA-0650 | |
| | GBT-IgA-0658 | |
| | GBT-IgA-0661 | |
| | GBT-IgA-0752 | |
| | GBT-IgA-0836 | |
| | GBT-IgA-0860 | |
| | GBT-IgA-0862 | |
| | GBT-IgA-1090 | |
| | GBT-IgA-1091 | |
| | GBT-IgA-1164 | |
| | GBT-IgA-1165 | |
| | GBT-IgA-1179 | |
| | GBT-IgA-1445 | |
| | GBT-IgA-1446 | |
| | GBT-IgA-1449 | |
| | GBT-IgA-1602 | |
| 58 | Anti-CD3 | GFTFSDYYMT |
| | epsilon CDRH1 | |
| 59 | Anti-CD3 | FIRNQARGYTSDHNPSVKG |
| | epsilon CDRH2 | |
| 60 | Anti-CD3 | DRPSYYVLDY |
| | epsilon CDRH3 | |
| 61 | Anti-CD3 | TSSQSLFNVRSQKNYLA |
| | epsilon CDRL1 | |
| 62 | Anti-CD3 | WASTRES |
| | epsilon CDRL2 | |
| 63 | Anti-CD3 | KQSYDLFT |
| | epsilon CDRL3 | |
| 64 | Anti-CD3 | EVQLVESGGG LVQPGGSLRL SCAASGFTFS DYYMTWVRQA PGKGLEWVAF |
| | Epsilon Heavy | IRNQARGYTS DHNPSVKGRF TISRDNAKNS LYLQMNSLRA EDTAVYYCAR |
| | Chain EFN EE | DRPSYYVLDY WGQGTTVTVS SASTKGPSVF PLAPSSKSTS GGTAALGCLV |
| | GBT-IgA-0661 | KDYFPEPVTV SWNSGALTSG VHTFPAVLQS SGLYSLSSVV TVPSSSLGTQ |
| | BMD-IgA-1156 | TYICNVNHKP SNTKVDKKVE PKSCEKTHTC PPCPAPEAAG APSVFLFPPK |
| | GBT-IgA-1164 | PKDTLMISRT PEVTCVVVDV SHEDPEVKFN WYVDGVEVHN AKTKPREEQY |
| | GBT-IgA-1165 | NSTYRVVSVL TVLHQDWLNG KEYKCKVSNK ALPAPIEKTI SKAKGQPREP |
| | GBT-IgA-1602 | QVYTLPPSRE EMTKNQVSLT CEVKGFYPSD IAVEWESNGQ PENNYKTTPP |
| | GBT-IgA-1623 | VLDSDGSFFL YSKLTVDKSR WQQGNVFSCS VMHEALHNHY TQKSLSLSPG |
| | | K |
| 65 | Anti-CD3 | DIQMTQSPSS LSASVGDRVT ITCTSSQSLF NVRSQKNYLA WYQQKPGKAP |
| | Epsilon Light | KLLIYWASTR ESGVPSRFSG SGSGTDFTLT ISSLQPEDFA TYYCKQSYDL |
| | Chain | FTFGGGTKVE IKRTVAAPSV FIFPPSDEQL KSGTASVVCL LNNFYPREAK |
| | GBT-IgA-0661 | VQWKVDNALQ SGNSQESVTE QDSKDSTYSL SSTLTLSKAD YEKHKVYACE |
| | BMD-IgA-1156 | VTHQGLSSPV TKSFNRGEC |
| | GBT-IgA-1164 | |
| | GBT-IgA-1165 | |
| | GBT-IgA-1602 | |
| | GBT-IgA-1623 | |
| 66 | Anti-CD3 | EVQLVESGGG LVQPGGSLRL SCAASGFTFS DYYMTWVRQA PGKGLEWVAF |
| | Epsilon | IRNQARGYTS DHNPSVKGRF TISRDNAKNS LYLQMNSLRA EDTAVYYCAR |
| | Variable | DRPSYYVLDY WGQGTTVTVS S |
| | Heavy | |
| | GBT-IgA-0661 | |
| | BMD-IgA-1156 | |
| | GBT-IgA-1164 | |
| | GBT-IgA-1165 | |
| | GBT-IgA-1602 | |
| | GBT-IgA-1623 | |
| 67 | Anti-CD3 | DIQMTQSPSS LSASVGDRVT ITCTSSQSLF NVRSQKNYLA WYQQKPGKAP |
| | Epsilon | KLLIYWASTR ESGVPSRFSG SGSGTDFTLT ISSLQPEDFA TYYCKQSYDL |
| | Variable | FTFGGGTKVE IK |
| | Light | |
| | GBT-IgA-0661 | |
| | BMD-IgA-1156 | |
| | GBT-IgA-1164 | |
| | GBT-IgA-1165 | |
| | GBT-IgA-1602 | |
| | GBT-IgA-1623 | |
| 68 | Constant | _ASTKGPSVF PLAPSSKSTS GGTAALGCLV KDYFPEPVTV SWNSGALTSG |
| | heavy EFN EE | VHTFPAVLQS SGLYSLSSVV TVPSSSLGTQ TYICNVNHKP SNTKVDKKVE |
| | Effector | PKSCEKTHTC PPCPAPEAAG APSVFLFPPK PKDTLMISRT PEVTCVVVDV |
| | function null | SHEDPEVKFN WYVDGVEVHN AKTKPREEQY NSTYRVVSVL TVLHQDWLNG |
| | mutations: | KEYKCKVSNK ALPAPIEKTI SKAKGQPREP QVYTLPPSRE EMTKNQVSLT |
| | L234A, L235A | CEVKGFYPSD IAVEWESNGQ PENNYKTTPP VLDSDGSFFL YSKLTVDKSR |
| | and G237A | WQQGNVFSCS VMHEALHNHY TQKSLSLSPG K |
| | (EU) | |
| | Heterodimerizing | |
| | mutations: | |
| | D221E, K409E | |
| | (EU) | |
| 69 | Anti-MIGIS-α | QVQLVESGGG VVQPGRSLRL SCAASGFTFS SYGMHWVRQA PGKGLEWVAV |
| | Heavy Chain | ISYDGSNKYY ADSVKGRFTI SRDNSKNTLY LQMNSLRAED TAVYYCAKLY |
| | Dual Fc | GGNPKDTIYH WGRGTLVTVS SASTKGPSVF PLAPSSKSTS GGTAALGCLV |
| | GBT-IgA-0834 | KDYFPEPVTV SWNSGALTSG VHTFPAVLQS SGLYSLSSVV TVPSSSLGTQ |
| | | TYICNVNHKP SNTKVDKKVE PKSCDKTHTC PPCPAPELLG GPSVFLFPPK |
| | | PKDTLMISRT PEVTCVVVDV SHEDPEVKFN WYVDGVEVHN AKTKPREEQY |
| | | NSTYRVVSVL TVLHQDWLNG KEYKCKVSNK ALPAPIEKTI SKAKGQPREP |
| | | QVYTLPPSRE EMTKNQVSLT CLVKGFYPSD IAVEWESNGQ PENNYKTTPP |
| | | VLDSDGSFFL YSKLTVDKSR WQQGNVFSCS VMHEALHNHY TQKSLSLSPG |
| 70 | Anti-MIGIS-α | EIVLTQSPAT LSVSPGERAT LSCRASQSVS SNLAWYQQKP GQAPRLLIYG |
| | Light Chain | ASTRATGIPA RFSGSGSGTE FTLTISSLQS EDFAVYYCQQ YNNWPPFTFG |
| | Dual Fc | PGTKVDIKRT VAAPSVFIFP PSDEQLKSGT ASVVCLLNNF YPREAKVQWK |
| | GBT-IgA-0834 | VDNALQSGNS QESVTEQDSK DSTYSLSSTL TLSKADYEKH KVYACEVTHQ |
| | | GLSSPVTKSF NRGECDKTHT CPPCPAPELL GGPSVFLFPP KPKDTLMISR |
| | | TPEVTCVVVD VSHEDPEVKF NWYVDGVEVH NAKTKPREEQ YNSTYRVVSV |
| | | LTVLHQDWLN GKEYKCKVSN KALPAPIEKT ISKAKGQPRE PQVYTLPPSR |
| | | EEMTKNQVSL TCLVKGFYPS DIAVEWESNG QPENNYKTTP PVLDSDGSFF |
| | | LYSKLTVDKS RWQQGNVFSC SVMHEALHNH YTQKSLSLSP G |
| 71 | Anti-MIGIS-α | QVQLVESGGG VVQPGRSLRL SCAASGFTFS SYGMHWVRQA PGKGLEWVAV |
| | Heavy Chain | ISYDGSNKYY ADSVKGRFTI SRDNSKNTLY LQMNSLRAED TAVYYCAKLY |
| | RR | GGNPKDTIYH WGRGTLVTVS SASTKGPSVF PLAPSSKSTS GGTAALGCLV |
| | GBT-IgA-0836 | KDYFPEPVTV SWNSGALTSG VHTFPAVLQS SGLYSLSSVV TVPSSSLGTQ |
| | (T18-NKp46 | TYICNVNHKP SNTKVDKKVE PKSCRKTHTC PPCPAPELLG GPSVFLFPPK |
| | bsAb) | PKDTLMISRT PEVTCVVVDV SHEDPEVKFN WYVDGVEVHN AKTKPREEQY |
| | | NSTYRVVSVL TVLHQDWLNG KEYKCKVSNK ALPAPIEKTI SKAKGQPREP |
| | | QVYTLPPSRE EMTKNQVSLT CLVKGFYPSD IAVEWESNGQ PENNYKTTPP |
| | | VLDSDGSFFL YSRLTVDKSR WQQGNVFSCS VMHEALHNHY TQKSLSLSPG |
| 72 | Anti-NKp46 | QVQLQQSGPE LVKPGASVKM SCKASGYTFT DYVINWGKQR SGQGLEWIGE |
| | Heavy Chain | IYPGSGTNYY NEKFKAKATL TADKSSNIAY MQLSSLTSED SAVYFCARRG |
| | EE | RYGLYAMDYW GQGTSVTVSS ASTKGPSVFP LAPSSKSTSG GTAALGCLVK |
| | GBT-IgA-0836 | DYFPEPVTVS WNSGALTSGV HTFPAVLQSS GLYSLSSVVT VPSSSLGTQT |
| | (T18-NKp46 | YICNVNHKPS NTKVDKKVEP KSCEKTHTCP PCPAPELLGG PSVFLFPPKP |
| | bsAb) | KDTLMISRTP EVTCVVVDVS HEDPEVKFNW YVDGVEVHNA KTKPREEQYN |
| | | STYRVVSVLT VLHQDWLNGK EYKCKVSNKA LPAPIEKTIS KAKGQPREPQ |
| | | VYTLPPSREE MTKNQVSLTC EVKGFYPSDI AVEWESNGQP ENNYKTTPPV |
| | | LDSDGSFFLY SKLTVDKSRW QQGNVFSCSV MHEALHNHYT QKSLSLSPG |
| 73 | Anti-NKp46 | DIQMTQTTSS LSASLGDRVT ISCRASQDIS NYLNWYQQKP DGTVKLLIYY |
| | Light Chain | TSRLHSGVPS RFSGSGSGTD YSLTINNLEQ EDIATYFCQQ GNTRPWTFGG |
| | GBT-IgA-0836 | GTKLEIKRTV AAPSVFIFPP SDEQLKSGTA SVVCLLNNFY PREAKVQWKV |
| | (T18-NKp46 | DNALQSGNSQ ESVTEQDSKD STYSLSSTLT LSKADYEKHK VYACEVTHQG |
| | bsAb) | LSSPVTKSFN RGEC |
| 74 | Anti-MIGIS-α | QVQLVESGGG VVQPGRSLRL SCAASGFTFS SYGMHWVRQA PGKGLEWVAV |
| | Heavy Chain | ISYDGSNKYY ADSVKGRFTI SRDNSKNTLY LQMNSLRAED TAVYYCAKLY |
| | RR | GGNPKDTIYH WGRGTLVTVS SASTKGPSVF PLAPSSKSTS GGTAALGCLV |
| | GBT-IgA-0650 | KDYFPEPVTV SWNSGALTSG VHTFPAVLQS SGLYSLSSVV TVPSSSLGTQ |
| | (T18-CD47 | TYICNVNHKP SNTKVDKKVE PKSCRKTHTC PPCPAPELLG GPSVFLFPPK |
| | bsAb) | PKDTLMISRT PEVTCVVVDV SHEDPEVKFN WYVDGVEVHN AKTKPREEQY |
| | | NSTYRVVSVL TVLHQDWLNG KEYKCKVSNK ALPAPIEKTI SKAKGQPREP |
| | | QVYTLPPSRE EMTKNQVSLT CLVKGFYPSD IAVEWESNGQ PENNYKTTPP |
| | | VLDSDGSFFL YSRLTVDKSR WQQGNVFSCS VMHEALHNHY TQKSLSLSPG |
| 75 | Anti-CD47 | QVQLVQSGAE VKKPGSSVKV SCKASGYTFS SYAISWVRQA PGQGLEWMGG |
| | Heavy Chain | ISPIFGTANY AQKFQGRVTI TADESTSTAY MELSSLRSED TAVYYCARDA |
| | EE | GRSSDVGWYV GAIDVWGQGT LVTVSSASTK GPSVFPLAPS SKSTSGGTAA |
| | GBT-IgA-0650 | LGCLVKDYFP EPVTVSWNSG ALTSGVHTFP AVLQSSGLYS LSSVVTVPSS |
| | (T18-CD47 | SLGTQTYICN VNHKPSNTKV DKKVEPKSCE KTHTCPPCPA PELLGGPSVF |
| | bsAb) | LFPPKPKDTL MISRTPEVTC VVVDVSHEDP EVKFNWYVDG VEVHNAKTKP |
| | | REEQYNSTYR VVSVLTVLHQ DWLNGKEYKC KVSNKALPAP IEKTISKAKG |
| | | QPREPQVYTL PPSREEMTKN QVSLTCEVKG FYPSDIAVEW ESNGQPENNY |
| | | KTTPPVLDSD GSFFLYSKLT VDKSRWQQGN VFSCSVMHEALHNHYTQKSLS |
| | | LSPG |
| 76 | Anti-CD47 | QSVLTQPPSA SGTPGQRVTI SCSGSSSNIG SNYVYWYQQL PGTAPKLLIY |
| | Light Chain | RNNQRPSGVP DRFSGSKSGT SASLAISGLR SEDEADYYCA AWDDSLSGVV |
| | GBT-IgA-0650 | FGGGTKLTVL GQPKAAPSVT LFPPSSEELQ ANKATLVCLI SDFYPGAVTV |
| | (T18-CD47 | AWKADSSPVK AGVETTTPSK QSNNKYAASS YLSLTPEQWK SHRSYSCQVT |
| | bsAb) | HEGSTVEKTV APTECS |
| 77 | MIGIS-α Full | DRLAGSCSVADWQMPPPYVVLDLPQETLEEETPGAN |
| | Length | |
| 78 | MIGIS-α | GSCCVADWQM PPPYVVLDLP QETLEEETPG AN |
| | peptide | |
| | 456C isoform | |
| | Shared with | |
| | human IgHA1 | |
| | IgHA2 | |
| 79 | hu IgA CH2- | HPRLSLHRPALEDLLLGSEANLTCTLTGLRDASGVTFTWTPSSGKSAVQ |
| | CH3-MIGIS-α | GPPERDLCGCYSVSSVLPGCAEPWNHGKTFTCTAAYPESKTPLTATLSK |
| | peptide | SGNTFRPEVHLLPPPSEELALNELVTLTCLARGESPKDVLVRWLQGSQE |
| | (456S | LPREKYLTWASRQEPSQGTTTFAVTSILRVAAEDWKKGDTFSCMVGHEA |
| | isoform) | LPLAFTQKTIDRLAGSCSVADWQMPPPYVVLDLPQETLEEETPGANLWP |
| | | T |
| 80 | MIGIS-α | GSSSVADWQM PPPYVVLDLP QETLEEETPG AN |
| | peptide | |
| | Cys deletion | |
| | for T18 | |
| | crystal | |
| | structure | |
| 81 | Anti MIGIS-α | GSCSVADWQM PPPYVVLDLP QETLEEETPG AN |
| | peptide | |
| | Variant used | |
| | for 15E7 | |
| | crystal | |
| | structure: | |
| 82 | IgA CH3 | QKTIDRLA |
| | terminal | |
| | residues | |
| | contiguous | |
| | with N | |
| | terminus of | |
| | MIGIS-α | |
| | variants in | |
| | T18 and 15E7 | |
| | crystal | |
| | structure | |
| 83 | linker | GGGGSGGGGSGGGGS |
| 84 | Long MIGIS-α | QKTIDRLAGS SSVADWQMPP PYVVLDLPQE TLEEETPGAN GGGGSGGGGS |
| | form | GGGGS |
| 85 | Short MIGIS-α | DWQMPPPYVVLDLPQETLEEETPGANGGGGSGGGGSGGGGS |
| | form | |

### EXAMPLES

In order that this invention may be better understood, the following examples are set forth. These examples are for purposes of illustration only and are not to be construed as limiting the scope of the invention in any manner.

### Example 1. Quantitation of Membrane IgA⁺ Levels on IgA⁺ RAMOS Overexpressing Cells and on IgA⁺ Primary B cells

The inventors hypothesized that in order to drive optimal cell kill, a depletion antibody must firstly engage effector cells possessing the horsepower to deliver potent cell kill on engaging low receptor copy numbers, and secondly the antibody must bind a necessary threshold number of receptors to support the effector mediated cell kill mechanisms. Based, on this, it was hypothesized that membrane IgA receptor number on specific B-cell populations could be a critical factor in therapeutic efficacy.

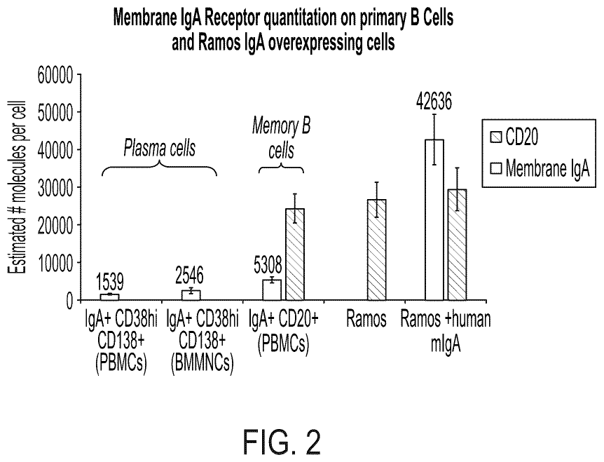

Quantitation of IgA receptor quantitation was determined using BD Quantibrite kit (PE Fluorescence Quantitation Kit). In summary, donor PBMCs and BMMNCs were stained with an in-house PE labeled anti-IgA Fc antibody termed 5F10 which is a high affinity antibody for human IgA Fc with K_{D} ˜92 pM. PE Quantibrite beads were used to determine the number of PE labelled antibodies bound per cell by flow cytometry following manufacturer's instructions (BD Biosciences). Briefly, 1×10⁵ cells/well were stained with PE labeled anti-IgA Fc antibody and cells were acquired using BD Fortessa. In this example membrane IgA molecules present on primary memory B cells (IgA⁺, CD20⁺) and plasma cells (IgA⁺, CD38⁺, CD138⁺) were compared to membrane IgA overexpressing Ramos cell lines. IgA producing plasma cells in PBMCs and BMMNCs were estimated to have in the range of approximately 1,500-2,500 copies per cell respectively while memory B cells had approximately 5,300 copies per cell (FIG. 2). CD20 is expressed on the surface of B-cells however expression is lost on terminally differentiated plasma cells. There was no CD20 expression detected on plasma cells (IgA⁺, CD38^{hi}, CD138⁺) isolated from BMMNC and PBMCs. The importance of membrane IgA copy number is highlighted in this example.

Based on these discoveries, it was hypothesized that a potent cell kill mechanism may be required to facilitate IgA⁺ B-cell depletion, to provide IgA⁺ B cell cytotoxicity on engagement of the low levels of membrane IgA presented on the target plasma cells and memory B cells. Overexpressing cell lines such as IgA Ramos presenting approximately 16-fold higher membrane IgA (FIG. 2) provide a critical mass of receptor numbers for effector engagement strategies to drive potent cell kill. In contrast, primary B cells, in particular plasma cells (IgA⁺, CD38hi, CD138⁺) isolated from BMMNCs and PBMCs, with membrane IgA levels at 1,500-2,500 copies per cell requires high a level of targeting antibody engagement with potent effector function to drive cell kill.

### Example 2. Derivation and Optimization of Anti-MIGIS-α Antibodies Providing Superior Binding to Membrane Presented Human IgA

Anti-MIGIS-a antibody (scFv) binders were identified from a human naïve library (WyHN5) panning campaign by phage display. In summary, the library was subjected to solution phase selection with reducing concentration of biotinylated MIGIS-a peptide SEQ ID NO: 77 over four rounds (round 1:200 nM, round 2:100 nM, round 3: 50 nM and round 4: 10 nM). A second panning branch was included where round 2 outputs from the peptide selected branch were used for input into the first of two additional rounds of selection on biotinylated human IgA CH2-CH3-MIGIS-a protein with MIGIS-a SEQ ID NO: 79, at a concentration of 200 nM in round 3 and 100 nM in round 4. Selections were conducted according to methods previously described (Finlay W J, et al., 2011, Methods Mol Biol., 681:383-401). Antibodies were screened using an enzyme-linked immunosorbent assay (ELISA) for binding to human MIGIS-a peptide SEQ ID NO: 77. The anti-MIGIS-a variable region designated HOS (SEQ ID NOs: 20 (VH) and 21 (VL)) with human germline sequence huIgHV3-30*18/huIGKV3-15*01 showed low non-saturating binding to IgA⁺ Ramos overexpressing cells. A second anti-MIGIS-a variable region called 15E7 with SEQ ID NO: 35 (VH) and 36 (VL) with human germline sequence huIgHV3-23*01/huIGKV3-11*01 also showed low non saturating binding to human IgA⁺ RAMOS cells, with heavy chain constant SEQ NO: 46, and human kappa constant SEQ ID NO: 47.

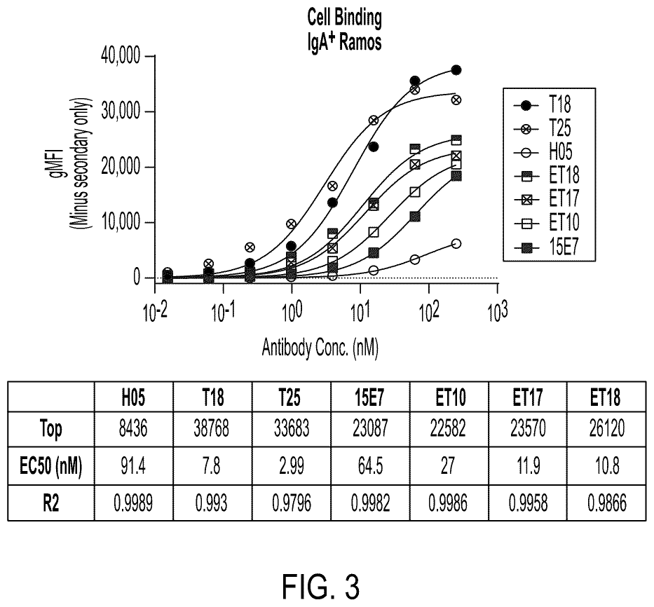

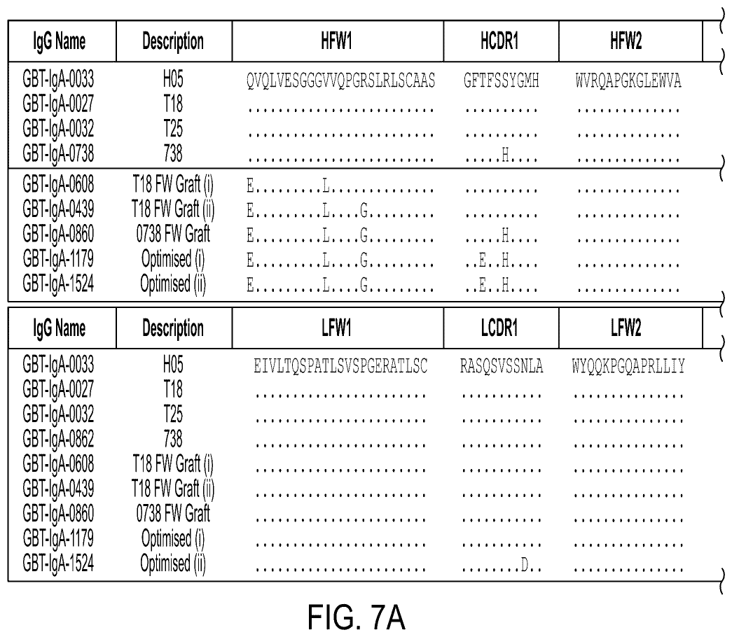

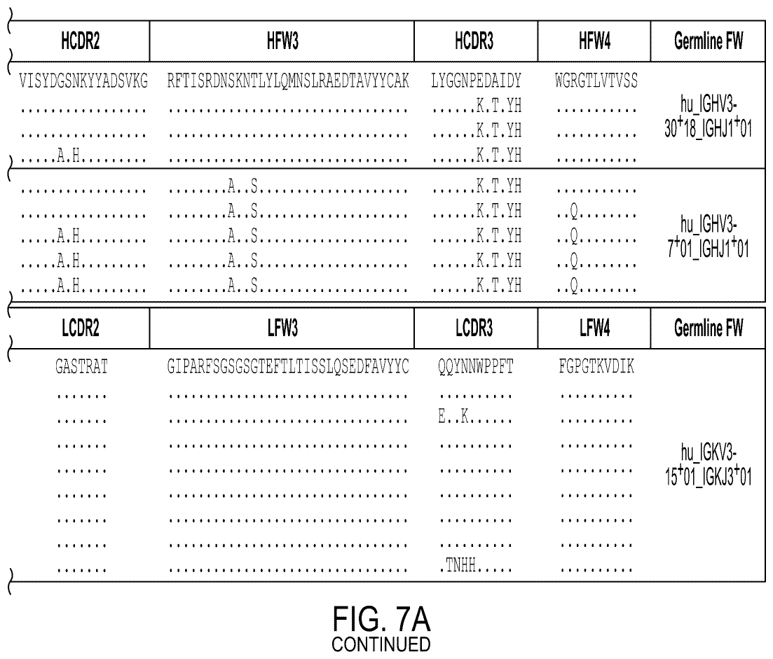

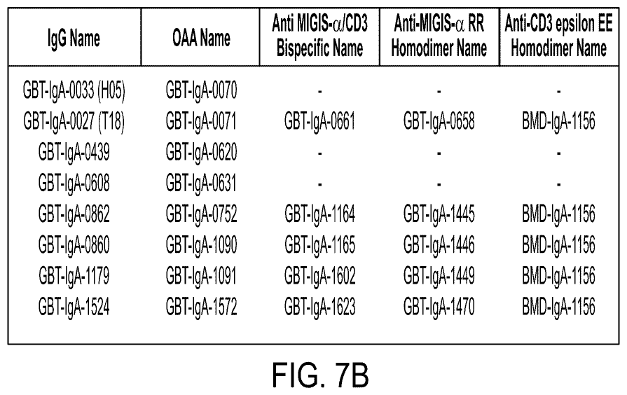

H05 and 15E7 were used as the basis for further optimization and engineering to enhance cell binding affinities and other critical properties. Optimization of HOS yielded two variants called T18 (GBT-IgA-0027, with variable regions comprising SEQ ID NO: 19 & 21) and T25 (GBT-IgA-0032, with variable regions comprising SEQ ID NO: 19 & 33) which showed significant improvement in binding on IgA⁺ Ramos cells over parental mAb (FIG. 3). HOS displays non saturating binding on IgA⁺ Ramos, whereas T18 displayed ˜4.6-fold increased MFI at the top saturating antibody concentration tested (250 nM), with an EC₅₀ of 7.8 nM. Saturating binding was also observed for T25 with an EC₅₀ of 2.9 nM and ˜4-fold MFI compared to H05. T18 has four variant positions in HCDR3 namely E101K, A103T, D105Y & Y106H (linear numbering), as shown in sequence alignments in FIG. 7A. No binding was observed on the parental (IgA⁻) Ramos cell line indicating specificity (data not shown).

Optimization of 15E7 yielded three variants: ET10 (comprising SEQ ID NO: 37 & 38), ET17 (comprising SEQ ID NO: 39 & 40), and ET18 (comprising SEQ ID NO: 41 & 42), all of which were prioritized due to improved binding on IgA⁺ Ramos cells over parental 15E7 (FIG. 3). Although the mean fluorescence intensity at the top saturating concentration tested for 15E7, ET10, ET17 and ET18 were similar (23,087, 22,582, 23,570 and 26,120 respectively), there is a significant left shift in binding curves for affinity matured variants with EC₅₀'s in the low double digit nM range for affinity matured variants (ET10: 27 nM, ET17:11.9 nM, ET18:10.8 nM). In comparison, 15E7 showed EC50 64.5 nM.

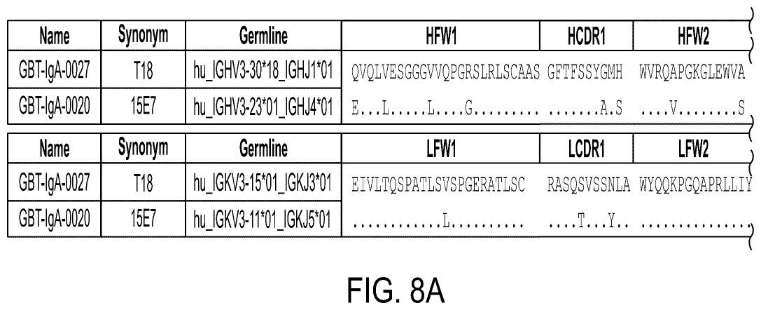

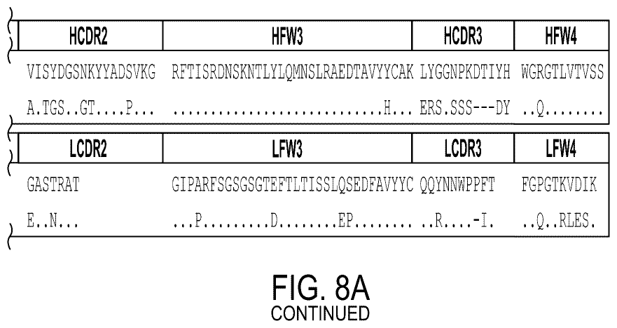

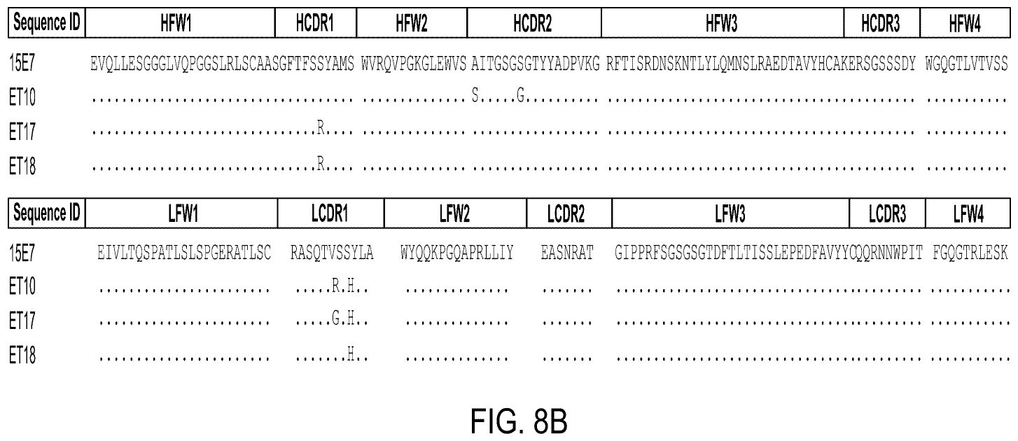

T18 and 15E7 variable heavy and light sequence differences are presented in FIG. 8A, while positional amino acid differences between the parental 15E7 and optimized variants are shown in FIG. 8 B. Similar to HOS and its variants, no binding to the parental (IgA⁻) Ramos cell line was observed indicating specificity (data not shown).

### Example 3. H05, 15E7 and Affinity Matured Variants Deliver Low Level Cytotoxicity (ADCC) Against Primary B Cells

T18, HOS and affinity matured variants were characterized for ADCC mediated cytotoxicity on primary B cells (CD19⁺, IgA⁺) isolated from two separate donors. PBMCs from 2 donors were isolated from fresh TRIMA using Ficoll gradient, and NK cells were further isolated using human NK isolation kit (StemCell Technologies, Cat #17955) following the manufactures instructions. Cells were harvested and washed in PBS before resuspending at 2×10⁶ cells/ml in pre-warmed complete media (RPMI and 10% FBS) containing 10 μM cell tracker violet (ThermoFisher, Cat #C10094) solution in. Cells were then incubated for 30 mins away from light at room temperature, before centrifugation (250×g for 4 min), and washing with PBS. Recovered cells were re-suspend in complete media at a concentration of 1×10⁶ cells/ml. Test antibody dilutions are prepared (2× final concentration in media) and 50 uL is added 50 uL cells aliquoted into 96 well round bottomed plates. The antibody/cell suspension is incubated for 15 minutes at 37° C. During incubation, NK cells are harvested by centrifugation (250×g for 4 minutes) and resuspend in RPMI+10% complete media, at 1.0×10⁶ cells per mL. To provide a final E:T ratio of 2:1 for example, 100 μl of effector cells are added to 100 μL of the cell/antibody suspension. Cells are incubated for 3 hours. For Annexin V and PI staining, cells are washed with Annexin Binding buffer (1×) (ThermoFisher, cat #. V13246) then centrifuged at 300×g for 5 minutes. Cells are resuspended in staining buffer with Annexin V488 (1:20) (ThermoFisher, cat #A13201) and PI (1:200) Propidium Iodide PE (Invitrogen, cat #006993-50). The suspension is incubated for 15 minutes in darkness at room temperature. Finally, cells are washed and resuspended in 100 μL Annexin Binding buffer. Samples were analyzed by flow cytometry. Cytotoxicity was measured as % of cells that are co-positive for Annexin V and propidium iodide, subtracting the % measured in non-treated background control.

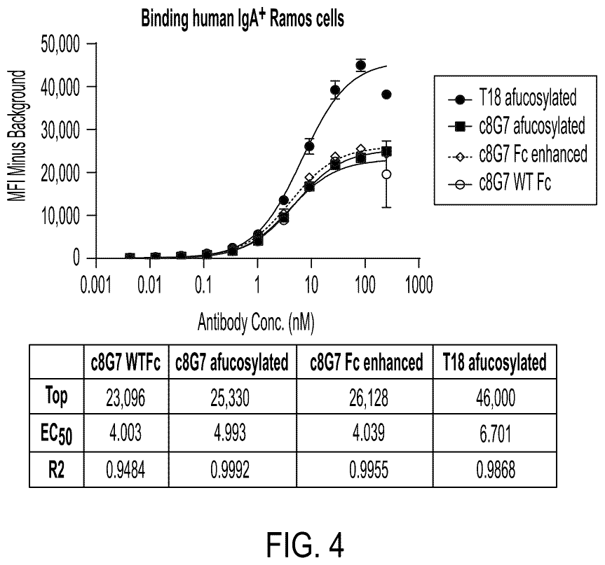

Primary B cell cytotoxicity data for 15E7 and variants (ET10, ET17 & ET18), and HOS and variants (T25 & T18) with effector enhancing mutations (S239D, I332E, A330L) layered on human IgG1 Fc (SEQ ID NO: 46) is shown in FIG. 4. At an antibody concentration 250 nM, mAbs 15E7, ET10, ET17, ET18 and HOS antibodies showed low level, s 10% cytotoxicity against primary B-cells. In contrast T18 and T25 showed modestly increased potency with cytotoxicity at approximately 25%. T18 was selected for further cytotoxicity profiling (human IgG1 Fc effector enhanced and afucosylated formats), revealed both formats have equivalent single digit nM potency against CD19⁺ IgA⁺ B cells with EC50 of 2.13 nM and 2.91 nM respectively. The maximum % cytotoxicity is also similar at 36.59% and 34.23% respectively with no cytotoxicity was observed against CD19⁺ IgA B cells (Table 3).

TABLE 3
Example showing NK ADCC on primary B-cells (IgA⁺,
CD19⁺) with T18 afucosylated and effector enhanced Fc.

| | Fc enhanced | Fc enhanced | Afucosylated | Afucosylated |
|---|---|---|---|---|
| | (CD19⁺ | (CD19⁺ | (CD19⁺ | (CD19⁺ |
| | IgA⁺) | IgA⁻) | IgA⁺) | IgA⁻) |
| Top % | 36.59 | No | 34.23 | No |
| cytotoxicity | | cytotoxicity | | cytotoxicity |
| EC₅₀ (nM) | 2.13 | — | 2.91 | — |
| R² | 0.985 | — | 0.991 | — |

### Example 4. Anti-MIGIS-α mAb T18 is Superior to c8G7 mAb in Binding to a Ramos Cell Line Overexpressing Membrane IgA

Cell binding comparisons between T18 (GBT-IgA-0027) and the benchmark mouse/human chimeric anti-MIGIS-α, antibody c8G7 (detailed in U.S. Pat. No. 9,688,776B2) were performed on Ramos cells overexpressing membrane IgA, (˜40,000 copies per cell as per FIG. 2). Afucosylated and Fc engineered antibodies were generated according to methods outlined in Zhong et al., Journal of Biotechnology, Volume 360, Pages 79-91, 2022. In summary, Ramos cells expressing human IgA (SEQ ID NO: 82 (heavy chain) and SEQ ID NO: 83 (light chain), both having leader SEQ ID NO: 87), were resuspended in cold PBS containing 2% BSA and treated with Fc block TruStain FcX reagent. Cells were incubated on ice with serial dilutions of anti-MIGIS-a mAbs for 2 h. Cells were then washed three times and surface stained with anti-human secondary PE antibody to detect bound antibody by flow cytometry. Results are shown in FIG. 4 with tabulated data presented in Table 4 below.

TABLE 4
Antibody binding to human IgA⁺ overexpressing RAMOS cells

| | c8G7 | c8G7 | c8G7 Fc | T18 |
|---|---|---|---|---|
| | WT Fc | afucosylated | enhanced | afucosylated |
| Top (MFI) | 23,096 | 25,330 | 26,128 | 46,000 |
| EC₅₀ (nM) | 4.0 | 4.90 | 4.04 | 6.70 |
| R² | 0.948 | 0.999 | 0.995 | 0.986 |

This example shows that T18 is a superior binder to IgA Ramos cells, binding a broader population of membrane IgA over c8G7 with increased signal intensity. Antibody c8G7 versions bind with similar MFI, whether WT Fc, afucosylated Fc, or possessing Fc enhancing mutations in the Fc, confirming that the nature of the Fc was not expected to impact binding.

### Example 5. Comparison of Anti-MIGIS-α mAb T18 to c8G7 in Selective Binding to IgA⁺ Cells

Anti-MIGISa mAb clone T18 is superior to anti-MIGISa mAb clone c8G7 in binding to Ramos cell line overexpressing membrane IgA.

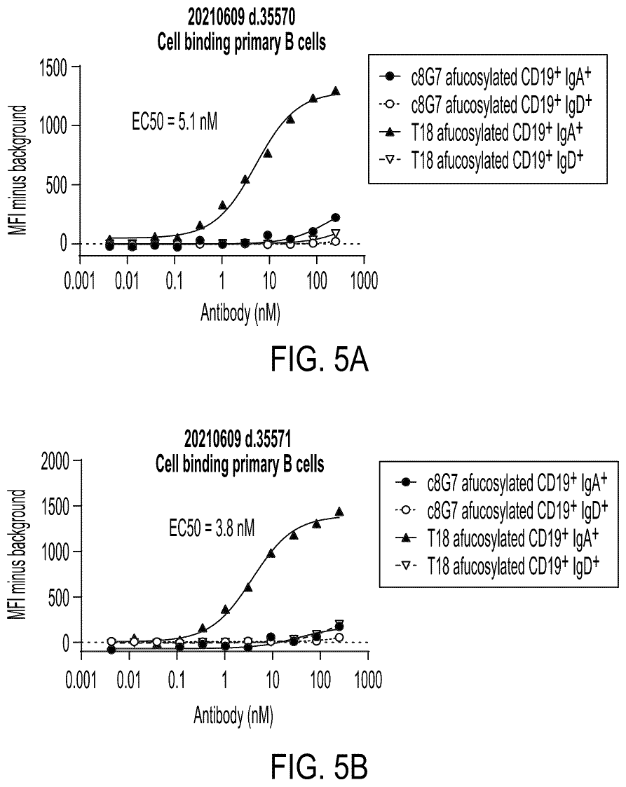

In this example, comparative binding of T18 and c8G7 (detailed in U.S. Pat. No. 9,688,776B2) with afucosylated huIgG1 Fc (SEQ ID NO: 46) on human IgA⁺ memory B cells was assessed. Cell binding assays were performed using primary human B cells isolated by negative selection from healthy adult PBMCs prepared from Trima residual (leukocyte enriched blood from apheresis collections) by centrifugation, using lymphoprep density gradients, RBC lysis and washing. Isolated B cells were resuspended in cold PBS+2% BSA and treated with Fc block TruStain reagent. Cells were incubated on ice with serial dilutions of anti-MIGIS-α antibodies for 2.5 hours. Cells were then washed thoroughly, and surface stained for IgA, IgD, CD19, and bound mAb for detection by flow cytometry. The MFI for detection of bound mAb was determined on CD19⁺ IgA⁺ IgD-populations (˜5,500 copies of membrane IgA estimated). The binding EC₅₀ for IgA⁺ primary human B cells is approximately 4-5 nM for T18 while 8G7 had non-saturable binding. Results were obtained from two biological donors of B cells shown below in Table 5 and in FIGS. 5A and 5B.

TABLE 5

| | Cell binding EC₅₀ (nM) on IgA⁺ | Peak MFI detected on IgA⁺ |
|---|---|---|
| mAb | CD19⁺ primary B cells | CD19⁺ primary B cells |
| c8G7 | not saturating, not saturating | 383, 175 |
| T18 | 5.1, 3.8 | 1287, 1393 |

T18 afucosylated showed binding EC50's of 3.8 and 5.1 nM on human (CD19⁺, IgA⁺ primary B cells from two exemplar donors, while c8G7 with identical afucosylated huIgG1 Fc (SEQ ID NO: 46), displayed non-saturable binding (Table 5). Specificity of binding is evident with no binding observed on CD19⁺, IgD⁺ B cell populations (CD19⁺, IgA⁺ and CD19⁺, IgD⁺). T18 is clearly a superior binder to c8G7 with saturable binding observed on memory B cells (CD19⁺, IgA⁺) presenting low IgA receptor numbers (˜5,300 copies per cell as per FIG. 2). T18 demonstrates increased binding over c8G7, with higher maximal MFIs observed on IgA Ramos (Table 4, T18 afucosylated MFI of 46,000 versus c8G7 afucosylated 25,330) and on primary B-cells (Table 5, T18 afucosylated MFI 1,287 versus c8G7 afucosylated 383.3). One explanation for the poorer binding profile for 8G7 over T18 on endogenously expressed membrane IgA from primary cells, is that c8G7 binding is occurring only on a small subset of membrane IgA possibly for steric or accessibility reasons.

### Example 6. Modality Mining Identified the Optimal Cell Depletion Mechanism to Deliver Increased Cell Kill Potency Against IgA Secreting Plasma Cells with Low Membrane IgA Copy Numbers

As discussed in Example 1, the inventors hypothesized that in order to drive optimal cell kill, a depletion antibody must engage effector cells to deliver potent cell kill and bind a necessary threshold number of receptors to support the effector mediated cell kill mechanisms.

In this example several cell depletion modalities were compared to establish what mechanism could deliver maximal cell kill. Example 3 shows the maximum % cytotoxicity (against IgA⁺ primary memory B-cells) achievable through ADCC mediated cell kill mechanisms is in the range 25-36% (leveraging T18 effector enhanced, and afucosylated Fc approaches). Consequently, alternative cell kill mechanisms were explored to identify a superior mechanism of action with increased cytotoxicity against target cells, despite expressing low levels of membrane IgA. A variety of depletion modalities were tested to augment antibody-dependent cellular cytotoxicity (ADCC), and antibody dependent cellular phagocytosis (ADCP). These included (i) NK cell engager approach via T18-NK-p46 bispecific (Patent WO 2015/197593 A1, (ii) combining afucosylated and dual Fc approach (Zhou F., Biochemistry. 2024, Apr. 16; 63(8):958-968) for increased avidity towards FcgR receptors with enhanced Fc effector function and (iii) an anti CD47 bispecific approach (blocking CD47-SIRPα interaction) to deliver enhanced phagocytic activity via antibody dependent cellular phagocytosis (ADCP). Additionally, a T cell retargeting mechanism was also evaluated leveraging a CD3 bispecific.

The T18 dual Fc antibody GBT-IgA-0834, comprises SEQ ID NO: 69 and 70 and was transiently expressed and purified from ExpiCHO-S in afucosylated format as outlined by Zhong et al., Journal of Biotechnology, Volume 360, Pages 79-91, 2022.

To enable T-cell retargeting cytotoxicity evaluation, the T18-CD3e bispecific antibody GBT-IgA-0661 comprised of the anti-CD3e binding arm with SEQ ID NOs: 64 and 65 (heavy & light chains respectively) and the anti-MIGIS-a binding arm with SEQ ID NO 56 and 57. The anti-CD3e variable heavy and light chain sequence is equivalent to CD3-0006 previously reported in patent US 2020/0010566 A1.

For the NK cell engaging approach the T18-NKp46 bispecific antibody GBT-IgA-0836 was generated, on huIgG1 with EE/RR heterodimerizing mutations, and leveraged the variable domains of NKp46-1 detailed in patent WO 2015/197593 A1, with anti-NKp46 heavy and light chain SEQ ID NO: 72 and 73 respectively, paired with anti-MIGIS-a SEQ ID NO: 71 and 57.

The T18-CD47 bispecific GBT-IgA-0650 comprised anti-CD47 heavy chain and light chains SEQ ID NO: 75 and 76 with variable regions previously described in parent WO 2021/124073 A1, paired with anti MIGIS-a heavy and light chains with SEQ ID NO: 74 and 57.

Additionally, a CD19-CD3 scFv (Cat number-Bimab-cd19cd3-01) obtained from Invivo Gen, 10515 Vista Sorrento Pkwy San Diego, CA 92121 USA was used as control. CD19 is expressed in all stages of B-cell development (apart from stem cells) but expression declines as they differentiate into mature plasma cells.

Bispecific antibodies with EE/RR heterodimerizing Fc mutations described herein, were purified using standard methods using a Protein A column (HiTrap MabSelect SuRe column). After elution the individual EE and RR homodimer pools were dialyzed into D-PBS pH7.2. Bispecific antibodies (EE/RR heterodimers) were generated by combining at a molar ratio of 1:1 EE Fc arms and RR arm Fc arms (1-20 mg/mL), in PBS, pH 7.4, and 15-25 mM cysteine was mixed together and incubated overnight (16 hours) at 25° C., followed by removal of the cysteine via dialysis, diafiltration, tangential flow filtration and/or spin cell filtration using standard methods. The formation of the bispecific antibody is analyzed by either cation exchange (CEX) HPLC, or hydrophobic interaction chromatography (HIC) HPLC. If desired, the bispecific antibody was polished by preparative CEX or HIC to remove residual parental(s).

IgA producing cells are typically present in very low frequency within PBMCs and cannot be reliably monitored by flow cytometry. For this reason, the levels of IgA in supernatants collected from cultured PBMCs is used as a surrogate readout for the number of surviving IgA producing cells (IgA⁺, CD38^{hi}, CD138⁺) in PBMCs treated with the test antibodies. The reduction in secreted IgA was detected using Thermo Fisher Scientific/Invitrogen's IgA ELISA kit (Cat Number: BMS2096) according to the manufacturer's instructions. Cryopreserved PBMCs were strained through a 37-micron filter after centrifugation in RPMI complete media after recovery. Cells with >97% viability were adjusted to a density of 10/mL. Aliquots (100 μL) were dispensed into wells of a 96 well U-bottom plate, and an equal volume with 2-fold the desired test antibody concentration(s) was added with mixing. The suspension was incubated at 37° C. 5% CO₂ without centrifugation. After 72-hour incubation the plate was centrifuged at 300× g for 5 minutes and supernatant was transferred to a new 96 well plate for IgA ELISA analysis.

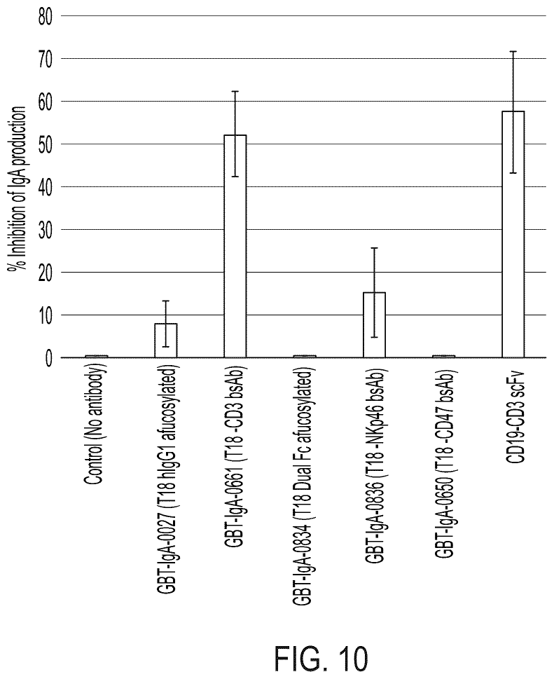

The mean percentage reduction of secreted IgA in supernatants treated with test depletion antibodies at 300 nM over untreated control is presented in FIG. 10. In this example GBT-IgA-0661 (T18-CD3 bispecific EFN EE/RR huIgG1) showed significantly greater potency activity against primary plasma cells, reducing levels of secreted IgA by 52% in PBMC supernatants over the untreated control, while CD19-CD3 treatment reduced IgA levels by 57%. In contrast, the T18-NKp46 bsAb (GBT-IgA-0836), reduced IgA levels by 15% while the T18 afucosylated mAb (GBT-IgA-0027) approximately 8% reduction. There was no reduction in IgA levels observed over untreated control for the afucosylated dual Fc and the T18-CD47 bispecific antibody formats. In an extension of this study, antibody titrations (with 5-fold serial dilution from 300 nM & CD19-CD3 with 10-fold serial dilution starting at 0.5 nM) were incubated with PBMCs (according to the methods described above). IgA levels in supernatants was then determined and data is presented in Table 6.

TABLE 6
Reduction in secreted IgA in human PBMC supernatants detected by
IgA ELISA following treatment with T18 leveraging alternative formats
to drive cytotoxicity against IgA secreting plasma cells.

| | GBT-IgA- | GBT-IgA- | GBT-IgA- | GBT-IgA- | GBT-IgA- | |
|---|---|---|---|---|---|---|
| | 0027 | 0661 | 0834 | 0836 | 0650 | |
| | T18 | T18-CD3 | T18 Dual Fc | T18 NKp46 | T18-CD47 | CD19-CD3 |
| | afucosylated | bispecific | afucosylated | bsAb | bsAb | scFv |
| Top (ng/ml) | 13.4 | 14.88 | 14.61 | 15.48 | 13.95 | 14.94 |
| EC₅₀ (nM) | No fit | 21.22 | No fit | 980.5 | No fit | 0.03677 |
| R² | No fit | 0.8167 | No fit | 0.1693 | No fit | 0.9063 |

GBT-IgA-0661 (T18-CD3 bispecific) showed EC50 of 21.22 nM while GBT-IgA-0836 (T18-NKp46 bispecific) showed EC50 980 nM on non-saturating fits. No fits could be established for the T18 afucosylated, T18 dual Fc afucosylated and T18-CD47 bispecific. Surprisingly, this example demonstrates that only the T-cell retargeting approach has the potency required to effectively deplete IgA⁺ plasma cells in PBMC supernatants due to low target receptor copy numbers.

Cytotoxicity against primary (IgA⁺, CD20⁺) memory B-cells was determined by flow cytometry in parallel, to confirm potency. PBMCs were strained through a 37-micron filter after centrifugation in RPMI complete media after recovery. Cells with >97% viability were adjusted to a density of 10⁶/mL. Aliquots (100 μL) were dispensed into wells of a 96 well U-bottom plate, and an equal volume with 2-fold the desired test antibody concentration(s) was added with mixing. The suspension was incubated at 37° C. 5% CO₂ without centrifugation. After 72-hour incubation the plate was centrifuged at 300×g for 5 minutes, supernatant removed, and cells were resuspended in PBS with Live/Dead near IR stain (1:1000) then incubated on ice for 30 mins. The plate was centrifuged at 300×g for 5 mins and PBS/stain removed. Cells were resuspended in 50 μL of PBS+0.5% BSA, 1 mM EDTA (MACs buffer) containing Fc block (1:10) then blocked for 5-10 mins at room temp prior to moving back on ice. Stain 2× (CD19-BV421 (1:10) and IgA-PE (1:25), in 50 μL), were added to the cells, followed by incubation on ice for 30-60 minutes. Cells were then centrifuged at 300×g for 5 minutes and supernatant removed followed by washing with 200 μL of MACs buffer with a final centrifugation step (300× g for 5 minutes). Finally, cells were resuspended in PBS with 0.5% PFA and analyzed by Fortessa. Cytotoxicity data is presented in Table 7.

TABLE 7
Cytotoxicity against IgA⁺ memory B cells by flow cytometry

| | IgA⁺ CD20⁺ B Cells | | | IgA⁻ CD20⁺ B Cells | |
|---|---|---|---|---|---|
| | GBT-IgA-0661 | GBT-IgA-0836 | | GBT-IgA-0661 | |
| | T18-CD3 | T18 NKp46 | CD19-CD3 | T18-CD3 | GBT-IgA-0836 |
| | bispecific | bsAb | scFv | bispecific | T18 NKp46 bsAb |
| Top (MFI) | 88.69 | 73.23 | 92.79 | No fit | No fit |
| EC₅₀ (nM) | 2.68 | 0.4568 | 0.003246 | — | — |
| R² | 0.9857 | 0.9891 | 0.9807 | — | — |

In this example cytotoxicity driven by GBT-IgA-0836 (T18-NKp46) with EC50 value of 0.45 nM (maximal MFI signal 92.79) compares favorably to GBT-IgA-0661 (T18-CD3 bispecific) which EC50 of 2.68 nM and maximal MFI signal 88.69. Cytotoxicity was also specifically directed to IgA⁺, CD20⁺ memory B-cells with no observed cell kill against IgA⁻, CD20⁺ memory B-cells.

A potent depletion mechanism is required considering the low receptor copy numbers presented on IgA secreting plasma B-cells displaying ˜1,500-2,500 copies membrane IgA. From this example, it is evident that the IgA copy number on IgA⁺ memory B cells (˜5,300 copies/cell, (FIG. 2)) can support NK mediated ADCC (via NK cell engagement) as well as T cell mediated cytotoxicity. In contrast however only the T cell retargeting approach provided an effective kill mechanism against IgA secreting (membrane IgA⁺) primary plasma cells (˜1,500-2,500 membrane IgA copies/cell).

### Example 7. Crystal Structure Analysis of Anti-MIGIS-α Antibodies

The crystallization of MIGIS-α peptide tethered to the N-terminus of VH-Fab-0027 (GBT-IgA-0027) and N-terminus of Fab-15E7 (GBT-IgA-0020) is described in this example. The resolved structure defines the key paratope/epitope interactions between T18 (GBT-IgA-0027) and the long MIGIS-α peptide. For the crystallization efforts, two constructs were generated: a long MIGIS-α form (SEQ ID NO: 84), comprising 8-residue fragment of IgA (SEQ ID NO: 82) at the N-terminus, followed by the MIGIS-α, sequence (SEQ ID NO: 80), and a linker (SEQ ID NO: 83). The long MIGIS-α form (SEQ ID NO: 84) was fused in-frame to the N-terminus of Fab of GBT-IgA-0027 (SEQ ID NO: 19). A short MIGIS-α form (SEQ ID NO: 85), comprising short-form MIGIS-α (residues 7-32 of SEQ ID NO: 81), and a linker (SEQ ID NO: 83) was fused in-frame to the N-terminus of Fab of GBT-IgA-0027 (SEQ ID NO: 19). The MIGIS-α-VH (long and short) was paired with the variable light chain with SEQ ID NO: 21.

Numbering of the MIGISα residues in this Example is according to SEQ ID NO: 84.

### A. Co-Crystallization of Fab-15E7 and a Chemically Synthesized Peptide Covering the MIGIS-α Peptide (SEQ ID NO: 31).

Co-crystals were obtained by hanging-drop vapor-diffusion method from a condition containing 10 mM zinc acetate and 20% PEG 3350. The crystals had symmetry consistent with the primitive space group P1. A data set to 3.1 Å resolution was collected from a single frozen crystal at IMCA 17-ID at the Argonne National Laboratory (APS), and the structure was determined by molecular replacement method.

### B. Crystallization of the MIGIS-α Peptide (SEQ ID NO: 31) Tethered to the N-Terminus of the VH Domain of Fab-0027.

A fusion protein comprising 8-residue fragment of IgA (SEQ ID NO: 82), MIGIS-α (SEQ ID NO: 77), a linker (SEQ ID NO: 83) and Fab of GBT-IgA-0027 (SEQ ID NO: 19) was crystallized by sitting-drop method from a condition containing 100 mM sodium Citrate pH 3.5 and 25% PEG 3350. The crystals had symmetry consistent with the orthorhombic space group P2₁2₁2₁. A data set to 1.88 Å resolution was collected from a single frozen crystal at IMCA 17-ID at the Argonne National Laboratory (APS), and the structure was determined by molecular replacement method.

### C. Structure Based Paratope/Epitope Residue Descriptors

The residues of the antigen and antibody are said to be hydrogen bonded if they include a hydrogen bond donor atom (bound to an electropositive hydrogen) in one molecule located within 3.2 Angstrom of a hydrogen bond acceptor atom having a lone pair of electrons in the other molecule. Residues of the antibody and antigen are said to form a salt bridge if they contain a positively charged atom in one molecule within 4 Angstrom of a negatively charged atom in the other molecule.

The per-residue solvent exclusion was determined by calculating the solvent accessible surface area of each residue of the antibody and antigen in complex and subtracting this from the sum of the solvent accessible surface areas of the two components considered individually. The solvent accessible surface area was calculated according to the method of Strake and Rupley (J Mol Biol 79 (2): 351-71, 1973). The pairwise buried surface area was used to estimate the individual contributions of pairs of residues from the antibody and antigen to the overall effect of buried surface area on binding energy. Since buried surface area is not pairwise decomposable, the buried surface area of each residue in the paratope was calculated in the presence of each individual antibody residue in the absence of the rest of the antibody. These individual contributions were then normalized so that the sum of all individual contributions of all antibody residues to the buried surface area of a given paratope residue would equal the total buried surface area of that paratope residue due to the binding of the entire antibody. This process was repeated in reverse for the individual contributions of paratope residues to the buried surface area of antibody residues. Residues are considered part of the paratope if the interaction results in a percentage buried surface area of >=20%.

Finally, any given residue is considered part of the paratope if it participates in a hydrogen bond with the antibody or with a water that is also hydrogen bonded to the antibody or if it participates in a salt bridge to a residue in the antibody or if it has a non-zero change in buried surface area due to interaction with the antibody. A residue in the antigen could be considered part of the epitope by similar criteria.

The paratope/epitope of the T18/MIGIS-α, (GBT-IgA-0027) and 15E7/MIGIS-α (GBT-IgA-0020) were determined using a structure-based descriptor (SBD) method. Briefly, the SBD method (see methods above) utilizes the crystal structure complex and determines which residues are significantly buried by the complex formation (>20% buried surface area (BSA)), residues that are participating in hydrogen bonds, residues participating in water mediate hydrogen bonds and residues participating in salt bridges, and residues that are in close contact (≤3.8 Angstroms). Residues that fit any of these criteria are determined to be the paratope and epitope. The paratope and epitope for T18/MIGIS-α interaction are shown in Tables 7 and 8.

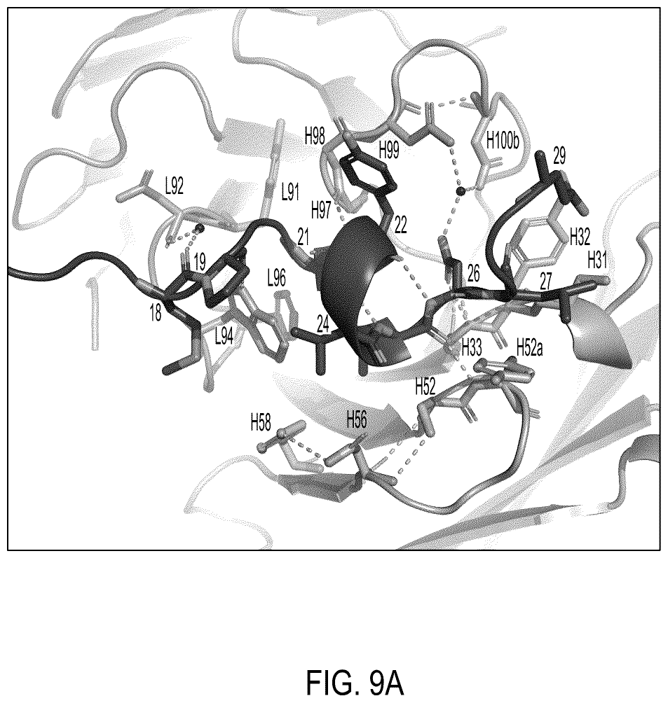

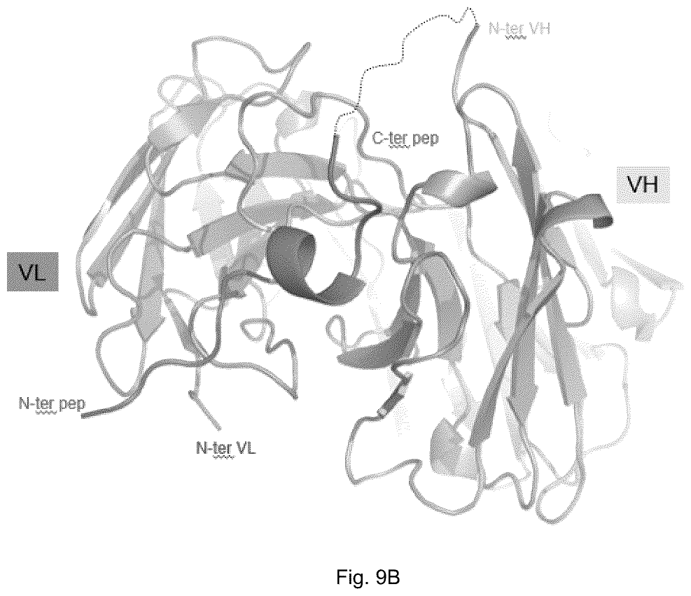

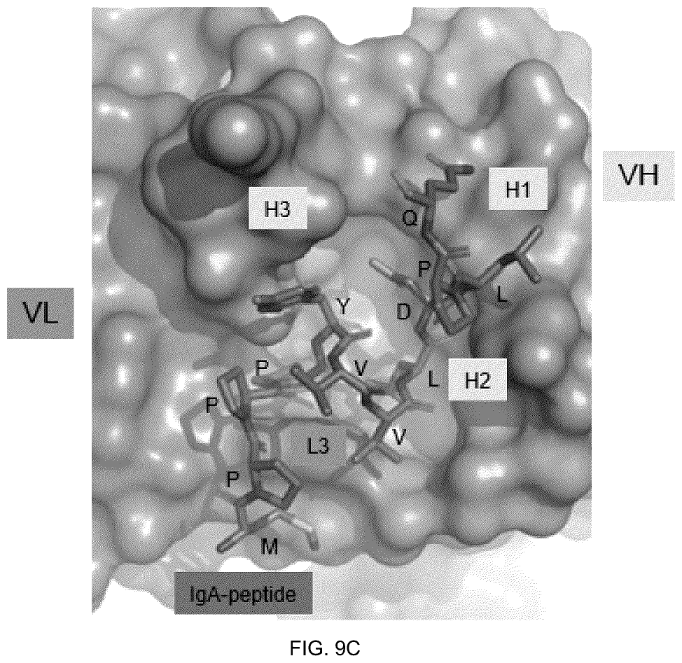

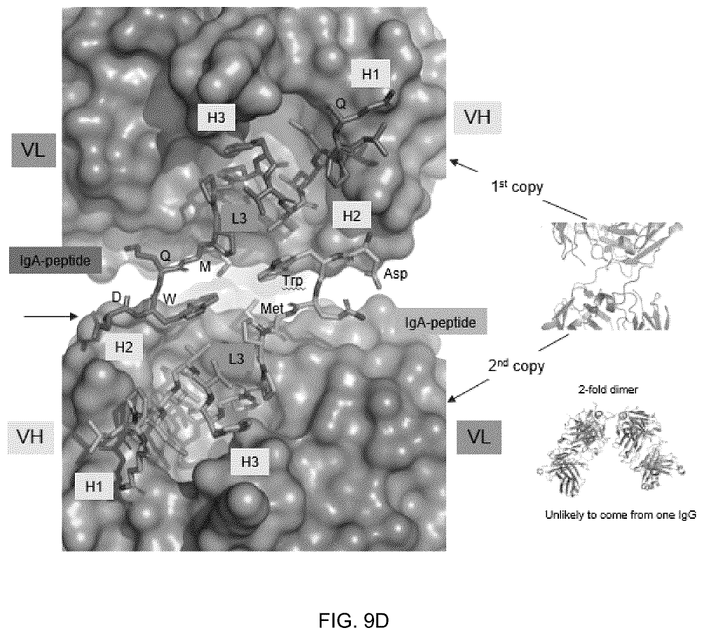

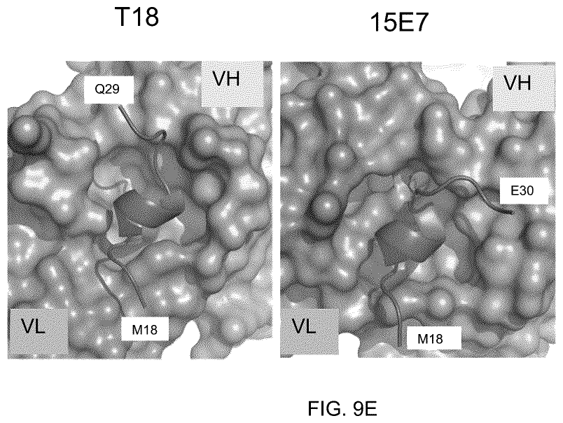

The main binding epitope and paratope for T18/GBT-IgA-0027 is the same for both Long Form and Short Form (compare FIG. 9B and FIG. 9C). 15E7 (GBT-IgA-0020) and T18 (GBT-IgA-0027) share a similar epitope: although the precise binding modes differ, the core epitope region is within the same linear stretch GBT-IgA-0020 (P19-Q29) and GBT-IgA-0027 (M18-Q29) and relies on similar residues. This MIGISα epitope region adopts the same conformation when bound by GBT-IgA-0020 or GBT-IgA-0027. The GBT-IgA-0020 and GBT-IgA-0027 binding epitope is preserved in other MIGIS-α isoforms, including MIGISα-456C (SEQ ID NO: 78), MIGISα-456S (SEQ ID NO: 81), and MIGISα-short form (residue numbers 7-32 of SEQ ID NO: 78). The binding epitope is closely conserved in cynomolgus monkeys, but diverges in rat and mouse, likely leading to slightly reduced binding affinity. The binding epitope is not found in the corresponding extracellular portion of MIGIS-γ, MIGIS-δ MIGIS-Σ, or MIGIS-μ.

The paratope and epitope for 15E7/MIGIS-α interaction are shown in Tables 10 and 11. For T18/MIGIS-α complex, the paratope residues are (Kabat notation), H31, H32, H33, H52, H52a, H56, H58, H97, H98, H99 and H100b, and the full suite of epitope residues are residues 18, 19, 21, 22, 24, 25, 26, 27, 28 and 29. For 15E7/MIGIS-α complex, the paratope residues are (Kabat notation), H31, H33, H50, H52, H52a, H58, H95, H96, H97, H98, L32, L91, L92, and L94, and the full suite of epitope residues are 19, 20, 21, 22, 24, 25, 26, 27, 28 and 29.

TABLE 8
Paratope sites for T18/MIGIS-α complex predicted by SBD

| | | | | |
|---|---|---|---|---|
| | | | Close contact | |
| | | | to Antigen | |
| | | Electrostatic | (>=3.8 | |
| Type | % BSA > 20 | Interaction | Angstroms) | Antigen Contacts |

Heavy Chain

| | | | | |
|---|---|---|---|---|
| S31 | 85.4 | HB | X | L25, D26, |
| | | | | L27, Q29 |
| Y32 | 59.5 | | X | L25, D26, |
| | | | | L27, Q29 |
| G33 | 95.5 | HB, H₂0-HB | X | L25, D26 |
| S52 | 99.2 | | X | V24, L25 |
| Y52a | 57.0 | HB | X | V24, L25, |
| | | | | D26, L27, |
| | | | | P28 |
| N56 | 29.6 | | | V24, L25 |
| Y58 | 32.2 | | | M18, V24, L25 |
| G97 | 83.4 | HB | X | P20, P21, Y22 |
| G98 | 49.3 | | X | P20, P21, Y22 |
| N99 | 56.1 | H₂0-HB | X | Y22, D26 |
| D100b | 44.1 | H₂0-HB | | Y22, D26 |

Light Chain

| | | | | |
|---|---|---|---|---|
| Y91 | | | X | M18, P21 |
| N92 | 35.2 | H₂0-HB | | M18, P19, |
| | | | | P20, P21 |
| W94 | 91.6 | HB | X | M18, P19, |
| | | | | P20, P21 |
| | | | | V24, V26 |
| F96 | | | X | P21, L25 |

HB: hydrogen bond; H₂0-HB: water mediated hydrogen bond; S-B: salt-bond.

TABLE 9
Epitope sites for T18/MIGIS-α complex predicted by SBD

| | | | Close contact |
|---|---|---|---|
| | | | to Antigen |
| | | Electrostatic | (>=3.8 |
| Site | % BSA > 20 | Interactions | Angstroms) |
| M18 | 39.1 | H₂0-HB | X |
| P19 | 11.6 | HB | X |
| P21 | 94.2 | | X |
| Y22 | 45.9 | HB | X |
| V24 | 64.2 | | |
| L25 | 95.8 | HB | X |
| D26 | 88.0 | HB, H₂0-HB | X |
| L27 | 46.3 | HB | X |
| Q29 | 21.6 | | X |

HB: hydrogen bond; H₂0-HB: water mediated hydrogen bond; S-B: salt-bond.

TABLE 10
Paratope sites for 15E7/MIGIS-α complex predicted by SBD

| | | | | |
|---|---|---|---|---|
| | | | Close contact | |
| | | | to Antigen | |
| Kabat | | Electrostatic | (>=3.8 | |
| Position | % BSA > 20 | Interactions | Angstroms) | Antigen Contacts |

Heavy Chain

| | | | | |
|---|---|---|---|---|
| S31 | 36.8 | | X | P28, Q29 |
| A33 | 100.0 | | X | V24, L25, Q29 |
| A50 | | | X | L25 |
| T52 | 66.7 | HB | X | V24, Q29, A31 |
| G52a | 100.0 | HB | X | Q29 |
| Y58 | 49.6 | | X | P19, V24, L25 |
| E95 | | | X | L25 |
| R96 | 52.1 | HB, S-B | X | Leu25, D26 |
| S97 | 89.4 | H-Bond | X | L25, D26, L27, Pro28, |
| | | | | Q29 |
| G98 | 36.9 | H-Bond | X | D26, L27, P28 |

Light Chain

| | | | | |
|---|---|---|---|---|
| Y32 | 42.7 | | X | Y22 |
| R91 | 80.0 | HB, S-B | X | P20, P21, Y22, L25, |
| | | | | D26 |
| N92 | 39.4 | | X | P20, P21, Y22 |
| W94 | 55.0 | | X | M18, P19, P20, P21, |
| | | | | V24, L25 |

HB: hydrogen bond; H₂0-HB: water mediated hydrogen bond; S-B: salt-bond.

TABLE 11
Epitopes sites for 15E7/MIGIS-α complex predicted by SBD

| | | | Close contact |
|---|---|---|---|
| | | | to Antigen |
| | | Electrostatic | (>=3.8 |
| Site | % BSA > 20 | Interactions | Angstroms) |
| P19 | 27.7 | | X |
| P20 | | | X |
| P21 | 99.2 | | X |
| Y22 | 59.8 | HB | X |
| V24 | 66.3 | | X |
| L25 | 99.7 | HB | X |
| D26 | 87.4 | HB, S-B | X |
| L27 | 4.3 | HB | X |
| P28 | 24.3 | | X |
| Q29 | 85.5 | HB | X |

HB: hydrogen bond; H₂0-HB: water mediated hydrogen bond; S-B: salt-bond.

In addition to identifying the residues involved in the epitope for the T18/MIGIS-α, and 15E7/MIGIS-α, these crystal structures were also used to identify important structural features of the MIGIS-α peptide involved in the binding interface. Using DSSP classification a key secondary structure features of the binding peptide was shown to be a helical region comprising residues P19, P21, Y22, V24, L25, D26, and L27, with coil or random coil regions surrounding. This helical region is at the center of the binding epitope for both T18 and 15E7 with significant numbers of close contacts and electrostatic contacts occurring at this region, and its presence in crystal structures for both antibodies suggests it is likely to be a feature of native MIGIS-α and not a crystallization artifact.

This helical epitope may explain the improved binding and other properties of T18 and 15E7 (and their derivatives) over some previously described anti-MIGIS-α antibodies which retain the ability to bind truncated MIGIS-α peptides lacking this helical region, indicating they bind to a different epitope (e.g. 8G7 & 29C11 in U.S. Pat. No. 9,688,776; 7C3, 9F5, 29C11 in Hung et al 2011; and KM4641 & KM4644 in Yamasaki et al, 2022).

Both antibodies GBT-IgA-0027 (T18) and GBT-IgA-0020 (15E7) bind a helical epitope comprising P19, P21, Y22, V24, L25, D26, and L27 of MIGIS-α. GBT-IgA-0027 (T18) (and its derivatives such as GBT-IgA-0032, GBT-IgA-0860, GBT-IgA-0862, GBT-IgA-1179, and GBT-IgA-1524, and their respective one-armed and bispecific versions) binds a helical epitope on MIGIS-a comprising P19, P21, Y22, V24, L25, D26, and L27. GBT-IgA-0032 (T18) also binds residues M18 and Q29.

GBT-IgA-0020 (15E7) (and its derivatives GBT-IgA-0034, GBT-IgA-0035, and GBT-IgA-0036 and their respective one-armed and bispecific versions) also bind this helical epitope on MIGIS-α comprising P19, P21, Y22, V24, L25, D26, and L27. GBT-IgA-0020 (15E7) also binds P20, P28, and Q29.

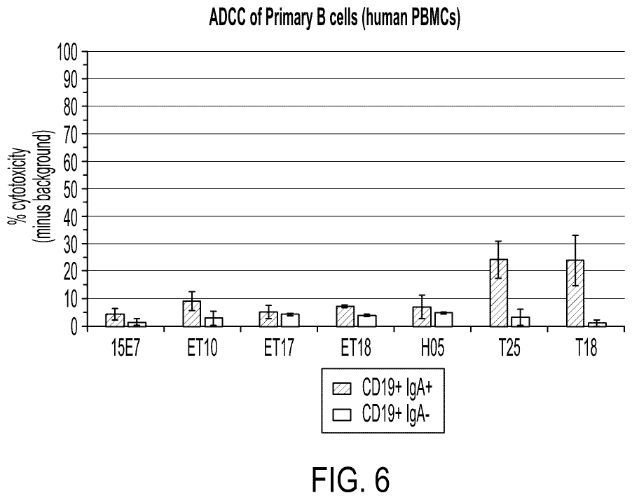

The CDR paratope residues of GBT-IgA-0027 (T18) and GBT-IgA-0020 (15E7) are highly conserved with each other, and across their immediate derivatives, (GBT-IgA-0032 (T25), GBT-IgA-0034 (ET10), GBT-IgA-0035 (ET17), and GBT-IgA-0036 (ET18)). FIG. 3 demonstrates that GBT-IgA-0027 (T18), GBT-IgA-0032 (T25), GBT-IgA-0035 (ET17), and GBT-IgA-0036 (ET18) all show improved binding over their respective parent antibodies (H05 for T18, T25, 15E7 for E10, E17, and E18), of ˜10 nM or less (ET10 improved, but by less). However, as demonstrated in FIG. 6, only GBT-IgA-0027 (T18) and GBT-IgA-0032 (T25) demonstrated increased potency, despite E17 and E18 having similar binding properties (the relatively poor binding of HOS shown in FIG. 3 is likely the reason it did not demonstrate potency in FIG. 6).

A comparison of CDR paratope residues for these antibodies shows many common residues that are likely involved in binding the shared helical core of P19, P21, Y22, V24, 125, D26, and L27:
CDR-H1-31 (S, T, N, Q, R, K, H, D, E,) a charged or polar residue;
CDR-H1-33 (A/G);
CDR-H2-52: (S or T);
CDR-H2-Y58;
CDR-H3-G98;
CDR-L3-N92;
CDR-L3-W94;

Further key paratope residues relevant to 15E7/MIGISα helix binding are: CDR-H2-A50, CDR-H2-G52a, CDR-H3-E95, CDR-H3-R96, CDR-H3-S97, CDR-L1-Y32, and CDR-L3-R91

The paratopes diverge at CDR-H3, suggesting that these residues may be relevant for the precise orientation of the anti-MIGISα antibody. Unlike a soluble target, MIGISα is relatively inaccessible, being fixed on the cell surface, limiting the steric opportunities for an antibody to bind MIGISα and have its CH3 domain correctly oriented for effector functioning (which may be critical, depending on the intended use). FIG. 9E shows a side-by-side comparison of the orientation of the T18/MIGISα complex (left panel, similar orientation as FIG. 9C) and the 15E7/MIGISα complex (right panel), where the different orientation of the MIGISα peptide to each antibody is apparent. This difference in orientation is likely caused by the differences in the CDR-H3 paratopes: CDRH3 of 15E7 binds to MIGISα through CDR-H3-95, CDR-H3-96, CDR-H3-97, and CDR-H3-98, which predominantly interact with MIGISα helix residues 125, D26, 127, as well as P28, and Q29. In contrast, the CDRH3 of T18 exclusively binds to binds to the MIGISα helix: at residues P20, P21, Y22, and D26. All 4 of the T18 CDR-H3 paratope residues interact with Y22, (see in FIG. 9A, 9C, and 9D).

Therefore, in addition to the above 7 paratope residues shared with 15E7, CDR-H3-G97, CDR-H3-G98, CDR-H3-N99, and CDR-H3-D100b appear important paratope residues for the T18 binding. Additional paratope residues for T18 are: CDR-H1-Y32, CDR-H2-H56; CDR-L3-Y91; and CDR-L3-F96.

### Example 8. Engineering of T18 for Affinity and Developability Characteristics

To drive maximum potency T18 was further engineered, yielding clone GBT-IgA-0862 (IgG format), which has three amino acid differences from the parental T18 variable heavy chain, namely in HCDR1 (S31H) and two in HCDR2 (G54A & N56H). For reference, sequence alignment of HOS, T18 and the key affinity optimized variants are presented in FIG. 7 A. GBT-IgA-0862 was further engineered to reduce elevated non-specificity scores, by introducing a single point mutation (T28E in HCDR1), in combination with heavy chain FW grafting into the huIgHV3-7*01 germline yielding clone GBT-IgA-1179. FIG. 7 B provides a reference table listing the various formats discussed herein and their identifiers. For example, GBT-IgA-1179 (IgG) format shares identical anti-MIGIS variable heavy and light chain sequence as the one-armed antibody GBT-IgA-1091, and CD3 bispecific molecule GBT-IgA-1602. Isomerization sequence liabilities in the parental HOS heavy chain variable region were successfully removed from Kabat positions 53 (HCDR2), and 101 (HCDR3) as well as a deamidation site at position 56 (HCDR2) in the context of the prioritized bispecific molecules GBT-IgA-1602 and GBT-IgA-1623. In addition, GBT-IgA-1623 also has a deamidation site sampled at position 92 (kabat) in LCDR3. Molecular assessment characterization of the top prioritized leads in CD3 bispecific format are presented Tables 12A and 12B.

### Tables 12A and 12B. Key In-Silico and In-Vitro Physicochemical Assessment of Prioritized Lead Anti-MIGIS-α/CD3 Bispecific Antibodies.

TABLE 12A

| | | | | NG-TCE | | |
|---|---|---|---|---|---|---|
| | Non Specificity Scores | | | Anti- | Anti- | Platform |
| | AC | | | MIGIS-α | CD3 | aSEC % |
| ANTIBODY | SINS | DNA | Insulin | arm | Arm | HMMS |
| GBT-IgA-1164 | 15 | 12 | 17 | 3 | 7 | 0.16 |
| GBT-IgA-1165 | 15 | 13 | 18 | 3 | 7 | 0.06 |
| GBT-IgA-1602 | 9 | 3 | 13 | 3 | 7 | 0.13 |
| GBT-IgA-1623 | 8 | 3 | 13 | 3 | 7 | 0.06 |

TABLE 12B

| | Thermal Stability (DSC) | | | | | | |
|---|---|---|---|---|---|---|---|
| | | Apparent | Charge heterogeneity | | | | huFcRn |
| | Tm1 | Tm Fab | | % | % | % | chromatography |
| ANTIBODY | (° C.) | (° C.) | iCE | Acidic | Main | Basic | RT (mins) |
| GBT-IgA-1164 | 72.95 | 76.18 | 9.1 | 16.9 | 72.5 | 10.6 | nd |
| GBT-IgA-1165 | 72.65 | 75.57 | 9.0 | 23.4 | 67.5 | 9.1 | nd |
| GBT-IgA-1602 | 69.91 | 75.47 | 9.1 | 0.8 | 99.2 | 0.0 | 1.4 |
| GBT-IgA-1623 | 69.91 | 75.41 | 0.1 | 0.6 | 99.3 | 0.1 | 1.8 |

This data shows that both GBT-IgA-1623 and 1602 offer improved non-specificity profiles (Avery et al., mAbs, 10(2), 244-255, 2018) over GBT-IgA-1164 and GBT-IgA-1602. For example, AC-SINS, DNA and insulin scores are reduced from 15, 12 and 17 (GBT-IgA-1164) to 9, 3 and 13 respectively in GBT-IgA-1602. In addition, charge heterogeneity assessment of the prioritized bispecific formats revealed that the % main peak is preferentially enhanced from 72.5% with GBT-IgA-1164 to 99% for GBT-IgA-1602 offering an improved developability profile. GBT-IgA-1623 & GBT-IgA-1602 were further assessed by huFcRn chromatography to determine column retention times. On assessment, GBT-IgA-1623 was shown to have elevated huFcRn retention time (1.8 minutes) over GBT-IgA-1602 (1.4 minutes). Antibodies with favorable retention times (less than 1.5 minutes) correlate with favorable PK profile in the human FcRn transgenic mouse models. Both bispecific antibodies were subsequently assessed in hFcRn transgenic mice (Tg32), and PK parameters were determined according to methods of Avery et al., mAbs, 8(6), 1064-1078, 2016). GBT-IgA-1602 has half-life ˜9 days (within expected ˜9-13-day half-life range for a human IgG1), while in line with huFcRn chromatography assessment, GBT-IgA-1623 showed atypical PK with 4.5 day half-life.

### Example 9. Determination of Binding Affinities of Anti MIGIS-α Antibodies (in One Arm Antibody Format) Against Human and Cyno MIGIS-α Peptides by Surface Plasmon Resonance (SPR)

This example determines the kinetics and affinity of various anti-MIGIS-α antibodies at 37° C. All experiments were performed on a BIAcore T200 surface plasmon resonance biosensor (Cytiva Global Life Sciences Solutions USA LLC 1209 Orange Street, Wilmington DE 19801, United States). The sensor chip surface (sample and reference flow cells) was initially conditioned with three consecutive 30 second pulses of 50 mM NaOH at a flow rate of 10 mL/min, followed by HBS-EP+ (10 mM HEPES, 150 mM NaCl, 0.05% v/v Surfactant P20, pH 7.4) buffer injection. Biotinylated human MIGIS-α peptide at a concentration of 1 mg/mL, was captured on the chip at a flow rate of 10 mL/min for 30 seconds (obtaining 290 RUs), while biotinylated cyno MIGIS-α peptide, was captured at a flow rate of 10 mL/min for 45 seconds at a concentration of 1 mg/mL (obtaining 284 RUs).

The analysis temperature for capture was 37° C. and the running buffer used was HBS-EP+. For multi-cycle kinetic analysis, the anti-MIGIS-α OAAs (analyte) were each diluted to a concentration of 5 nM in HBS-EP+, with 2-fold serial dilutions over 5 concentrations. Analyte was injected at a flow rate of 70 mL/min with association and dissociation times of 120 seconds and 180 seconds respectively. Chip regeneration was achieved by flowing 10 mM NaOH for 30 seconds at 10 mL/min. The sensorgrams were double-referenced and fit to a 1:1 Langmuir kinetics model using Biacore Insight Evaluation 3.0.12.15655 (Cytiva Global Life Sciences Solutions USA LLC 1209 Orange Street, Wilmington DE 19801, United States). Kinetics and affinity parameters are shown in Table 13. GBT-IgA-0070 (H05 in one arm antibody format), with apparent KD ˜210 nM for human MIGIS-α peptide (and ˜56 nM for cyno MIGIS-α peptide) was affinity optimized to deliver the prioritized clone T18. T18 in one armed format (GBT-IgA-0071), delivered approximately 20-fold improved affinity for human MIGIS-α (app KD 10.7 nM), and 5-fold improved binding against cyno MIGIS-α (app KD 11.9 nM). T18 was further affinity optimized, leading to generation of GBT-IgA-0862. In one armed format (GBT-IgA-0752), this delivered ˜16-fold affinity gains over T18 with KD of 0.665 nM for human MIGIS-α. Similar affinity improvements were achieved on cyno MIGIS-α peptide with ˜14-fold affinity gain, with app KD of 0.795 nM. GBT-IgA-0752 was further optimized for developability characteristics (outlined in Example 8) with a slight affinity cost generating the lead molecule GBT-IgA-1091 (OAA name), with KD measured at 2.1 nM and 2.5 nM against human & cyno MIGIS-α peptide respectively (Table 13). Binding characterization by SPR of the anti-CD3 binding arm (h2B5v6) in BMD-IgA-1156 had apparent KD 17 nM on recombinant human CD3 epsilon.

TABLE 13
Determination of binding affinities and kinetic constants of affinity
matured anti MIGIS-α antibodies (OAA format) against human
and cyno MIGIS-α peptides by Surface Plasmon Resonance (SPR).

| One Armed | Human MIGIS-α | | | Cyno MIGIS-α | | |
|---|---|---|---|---|---|---|
| Antibody Name | ka (1/Ms) | kd (1/s) | KD (M) | ka (1/Ms) | kd (1/s) | KD (M) |
| GBT-IgA-0070 | 1.05E+05 | 0.0222 | 2.10E−07 | 2.75E+05 | 0.0154 | 5.60E−08 |
| GBT-IgA-0071 | 2.96E+06 | 0.0316 | 1.07E−08 | 1.10E+06 | 0.0132 | 1.19E−08 |
| GBT-IgA-0752 | 2.29E+06 | 0.0015 | 6.65E−10 | 2.22E+06 | 0.0017 | 7.95E−10 |
| GBT-IgA-1090 | 2.06E+06 | 0.0015 | 7.244E−10 | 1.27E+06 | 0.0013 | 1.05E−09 |
| GBT-IgA-1572 | 8.18E+05 | 0.0028 | 3.47E−09 | 5.48E+05 | 0.0018 | 3.40E−09 |
| GBT-IgA-1091 | 1.32E+06 | 0.0028 | 2.13E−09 | 9.53E+05 | 0.0023 | 2.46E−09 |

### Example 10. In Vitro Binding of GBT-IgA-1602 on IgA Ramos and Primary IgA⁺ B-Cells Isolated from PBMCs

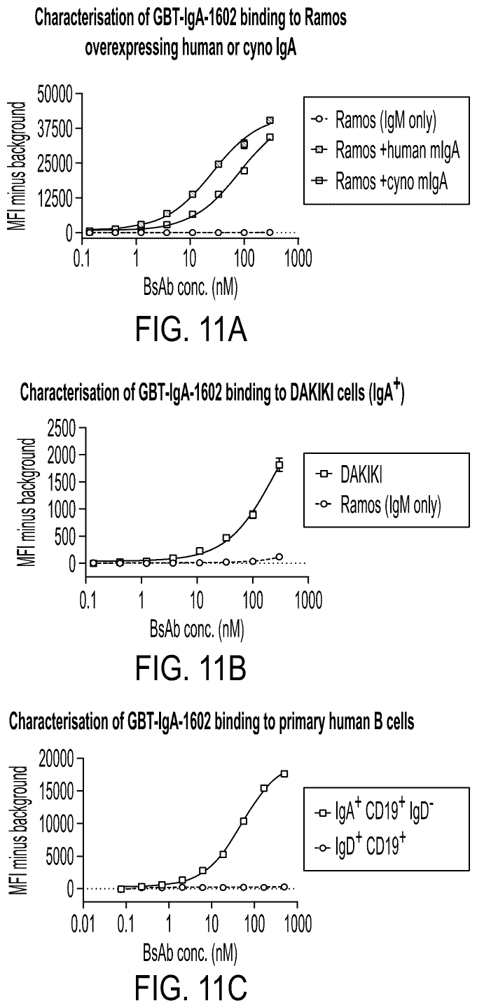

Cell binding assays were performed to measure the binding EC₅₀ and selectivity of GBT-IgA-1602, the prioritized anti-MIGIS-α/CD3 bispecific antibody. Ramos cells overexpressing either human or cynomolgous monkey membrane IgA (>40,000 copies per cell estimated by BD Quantibrite kit) were tested alongside the parental Ramos cell line, which expresses only cell surface IgM. The DAKIKI B cell line with low endogenous expression of mIgA (˜2,700 copies per cell estimated) was also evaluated. Serial dilutions of GBT-IgA-1602 were incubated on ice with each cell line for 2 hours before washing thoroughly and staining cells for the detection of the bound bispecific antibody by flow cytometry. The binding EC50 of GBT-IgA-1602 for Ramos +human mIgA cells is approximately 40 nM (FIG. 11A). While clear above-background binding is observed for Ramos +cyno mIgA (FIG. 11A) and DAKIKI cells (FIG. 11B) the dose-response is not saturating and reliable EC50s cannot be accurately determined. GBT-IgA-1602 bound to mIgA negative Ramos cells only marginally above background at the highest antibody concentration tested demonstrating selectivity for mIgA expressing cells (FIG. 11B).

Cell binding assays were performed using primary human B cells isolated by negative selection from healthy adult PBMCs prepared from Trima residual (leukocyte enriched blood from apheresis collections) by centrifugation using lymphoprep density gradients, RBC lysis and washing. After 3 h incubation on ice with serial dilutions of GBT-IgA-1602 cells were washed thoroughly and surface stained for IgA, IgD, and for detection of bound BsAb by flow cytometry. (Note: the antibody detecting surface IgA, clone IS11-8E10 from Miltenyi (Miltenyi US Headquarters, 1201 Clopper Road, Gaithersburg, MD 20878, USA), does not compete with our MIGIS-α binding antibodies, including GBT-IgA-1602). The binding EC₅₀ for IgA⁺ primary human B cells (˜5,500 copies per cell estimated) is approximately 50 nM and minimal above background binding was detected on IgD cells (FIG. 11C).

As presented in Table 14, the binding EC50 of GBT-IgA-1602 for human membrane IgA+ Ramos cells is approximately 40 nM. This value represents the mean EC50 value form three separate experiments. It is worth noting that the bispecific antibody is monovalent for anti-MIGIS-a (a single binding arm), therefore was expected to show a binding (EC50 shift) than the bivalent afucosylated IgG format presented in Table 4. Only background level binding was observed to parental Ramos confirming selectivity for membrane IgA expressing cells.

In addition, cell binding assays on primary human B cells isolated from three individual donors by negative selection from healthy adult PBMCs prepared from Trima residual (leukocyte enriched blood from apheresis collections) by centrifugation using lymphoprep density gradients, RBC lysis and washing. After 3 h incubation on ice with serial dilutions of GBT-IgA-1602 cells were washed thoroughly and surface stained for IgA, IgD, and for detection of bound bispecific antibody by flow cytometry. The Miltenyi antibody IS11-8E10 (Miltenyi US Headquarters, 1201 Clopper Road, Gaithersburg, MD 20878, USA), was used to confirm the presence of membrane IgA on cell. This antibody does not compete for membrane IgA with MIGIS-α binding antibodies. The binding EC₅₀ for IgA⁺ primary human B cells (˜5,500 copies per cell estimated) is approximately 50 nM and minimal above background binding was detected on IgD cells (not captured in Table 14).

TABLE 14
Key in vitro attributes of GBT-IgA-1602 from cell-based assays
EC50 values in nM for each assay are means ± standard deviations with the
number of biological replicates or healthy PBMC donors in parentheses.

| | | Primary cell killing, potency EC₅₀ (nM) | | | |
|---|---|---|---|---|---|
| Cell binding EC₅₀ (nM) | | Human | Human | | Cyno soluble |
| | Primary human | memory B cells | Plasma Cells | Cyno memory | Plasma Cells |
| Human IgA⁺ | IgA⁺ memory | (membrane | (IgA | membrane | (IgA |
| Ramos | B cells | IgA+) | secreting) | IgA+ | secreting) |
| 40.55 ± 22.19 | 49.8 ± 1.35 | 0.73 ± 0.47 | 1.67 ± 1.29 | 0.19 ± 0.06 | 1.34 ± 0.06 |
| (3) | (3) | (15) | (11) | (2) | (2) |

### Example 11. Ex Vivo Cytotoxic Activity of GBT-IgA-1602 and GBT-IgA-1164 in Primary Human PBMCs. Evidence of Selective Cell Killing Potency Against Human IgA Plasma Cells/Plasmablasts

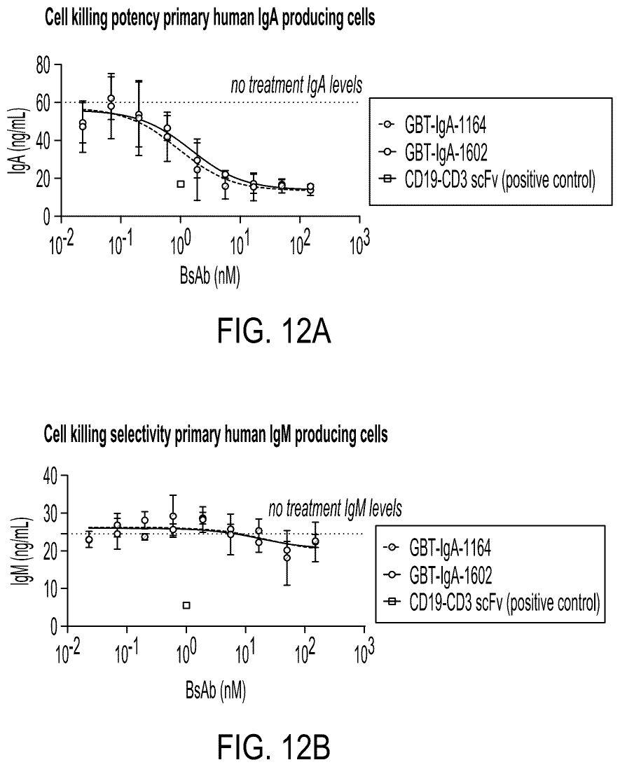

Potency assays were performed with healthy adult PBMCs isolated as described above. The PBMCs were not subjected to cell specific enrichment or depletion methods. Instead, the PBMCs used in these potency assays contain their natural proportions of immune cells and contain both the effector T cells and target cell populations: memory B cells (mIgA⁺ CD19/CD20⁺) and soluble IgA producing cells (mIgA⁺ CD20⁻ CD38^{hi} CD138^{-/+}). Serial dilutions of BsAbs were prepared and incubated with PBMCs cultured in wells of 96 well U-bottom plates (10⁶ cells per well). IgA producing cells are typically present in very low frequency within PBMCs and cannot be reliably monitored by flow cytometry. For this reason, we measured the concentration of IgA in supernatants collected from cultured PBMCs as a surrogate readout for the number of surviving IgA producing cells after 67-72 hours of treatment at 37° C. IgA levels detected by ELISA or MSD assays were reduced in a dose-dependent manner with GBT-IgA-1602 treatment and the maximal extent of this decrease matched the effect induced by the positive control blinatumomab (CD19-CD3 scFv), which broadly targets CD19⁺ B cells and plasma cells. Both GBT-IgA-1602 and GBT-IgA1164, display single-digit nM EC50s (FIG. 12A). IgM levels were reduced only with treatment with the positive control, highlighting the ex vivo selectivity of GBT-IgA-1602 and GBT-IgA-1164 for IgA producing cells (FIG. 12B). Note, the anti-MIGIS-α arm of GBT-IgA-1164 (huIGHV3-30*18 germline), differs from GBT-IgA-1602 (human IGHV3-7*01 germline) by 7 amino acids in total, located in HFW1 (Q1E, V11L, R16G), CDRH1 (T28E), HFW3 (S74A & T77S) and HFW4 (R109Q) (IMGT germline nomenclature with linear sequence numbering). The anti-MIGIS-a light chain and anti-CD3 heavy and light chains are identical in GBT-IgA-1164 and GBT-IgA-1602.

### Example 12. Evidence of Selective Cell Killing Potency Against Human mIgA⁺ Memory B Cells

Cell killing of mIgA⁺ memory B cells was measured from treated PBMCs in parallel by cell staining and flow cytometry. Counts of live mIgA⁺ CD19⁺ IgD⁻ events were normalized to total singlets of the gated lymphocytes and treatments compared relative to no treatment control:

$$\% cytotoxicity = {( 1 - ( \# of live {mlgA}^{+} CD {19}^{+} {lgD}^{-} cells @ test dose / \# of live {mlgA}^{+} CD {19}^{+} {lgD}^{-} cells @ no treatment ) )}^{*} 100$$

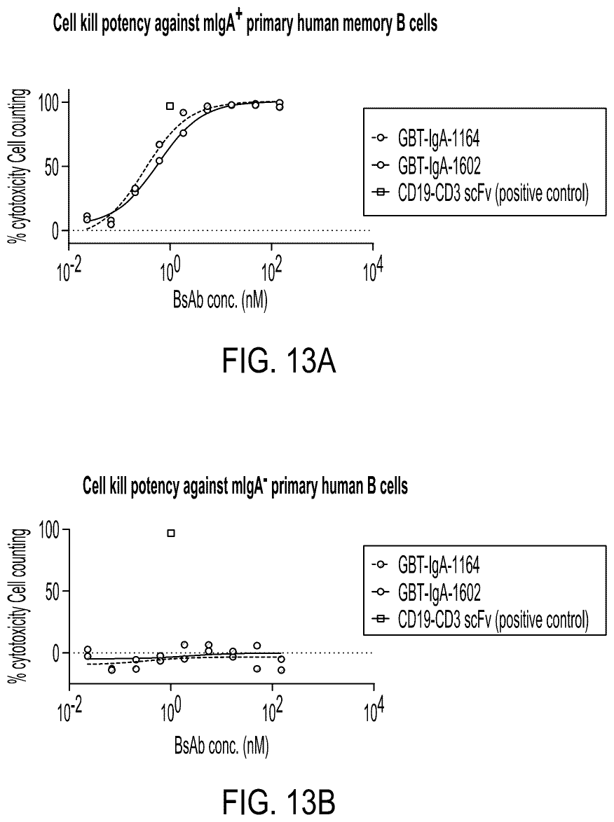

Both GBT-IgA-1602 and GBT-IgA-1164 induced killing of mIgA⁺ memory B cells with comparable sub nM EC₅₀ s (FIG. 13A). When mIgA⁻ CD19⁺ cells were monitored cytotoxicity was observed only with CD19-CD3 scFv positive control treatment; GBT-IgA-1602 exhibits high selectivity for the intended target cells. (FIG. 13B). In some experiments, mIgA⁻ CD19⁺ cells increased in numbers (˜20-50%) with the higher concentrations of GBT-IgA-1602 and GBT-IgA-1164. Due to the inconsistency of this observation with repeated analysis of the same biological donors its significance is unclear.

GBT-IgA-1602 exhibited selective potency against mIgA⁺ memory B cells and IgA producing cells within cynomolgus monkey PBMCs (isolated and locally provided fresh by iQ Biosciences, 1640 South Loop Rd., Suite 100, Alameda, CA 94502, US) with EC₅₀ s comparable to results with human PBMCs (Table 14). Note, EC50 values in nM for each assay are means±standard deviations with the number of biological replicates or healthy adult PBMC donors in parentheses. GBT-IgA-1602 showed sub nanomolar potency against human (EC50 0.73 nM), representing average value from 15 donors, and cyno memory B cells (EC50 0.19 nM) from 2 donors. When IgA levels were also assessed by ELISA, a dose-dependent reduction in IgA was observed with increasing concentration of GBT-IgA-1602. In data, not captured in the table, treatment by the positive control blinatumomab (CD19-CD3 scFv) at a concentration of 1 nM, matched the maximal extent of the decrease effect induced by the anti-MIGIS-α/CD3 bispecific antibodies tested. Blinatumomab broadly targets all CD19⁺ B cells and plasma cells.

In a separate analysis, GBT-IgA-1602 and the pre-optimized bispecific antibody GBT-IgA-1164 were assessed for cytotoxicity against memory B-cells and IgA producing cells from a single donor. The bispecific, GBT-IgA-1164 is represented by GBT-IgA-0752 in one armed antibody format which was determined to have has higher affinity for human and cyno MIGIS-a (app K_{D} ˜665 pM and 795 pM respectively) than GBT-IgA-1091, the one armed antibody version of GBT-IgA-1602 (app K_{D} 2.1 nM and 2.5 nM for human and cyno MIGIS-α, respectively). Despite the approx. 3-fold affinity difference, both GBT-IgA-1602 and GBT-IgA-1164, display similar pM EC50s, in IgA memory B cell kill assays with exemplar data from a single donor shown in Tables 15A and 15B.

Note, the anti-MIGIS-α arm of the pre-optimized antibody GBT-IgA-1164 (huIGHV3-30*18 germline), differs from GBT-IgA-1602 (human IGHV3-7*01 germline) by 7 amino acids in total shown in FIG. 7A. These are located in HFW1 (Q1E, V11L, R16G), CDRH1 (T28E), HFW3 (S74A & T77S) and HFW4 (R105Q) (Pfabaq| numbering, detailed in FIG. 1). The anti-MIGIS-α light chain and anti-CD3 heavy and light chains are identical in GBT-IgA-1164 and GBT-IgA-1602.

Tables 15A and 158. Exemplar Data Showing Anti MIGIS-α/CD3 Bispecific Induced Cell Kill Potency (EC50) Against IgA⁺ Memory B-Cells and IgA Producing Cells from Donor PBMCs.

TABLE 15A
IgA⁺ Memory B cell kill

| | GBT-IgA-1164 | GBT-IgA-1602 |
|---|---|---|
| Top | 100.8 | 100.9 |
| (% cytotoxicity) | | |
| EC50 (nM) | 0.318 | 0.587 |
| R² | 0.978 | 0.995 |

TABLE 15B
IgA producing B cell kill

| | GBT-IgA-1164 | GBT-IgA-1602 |
|---|---|---|
| Top (ng/ml) | 13.37 | 13.82 |
| EC50 (nM) | 0.916 | 1.504 |
| R² | 0.759 | 0.825 |

### Example 13 Select Germline Testing

The following heavy and light germline frameworks (with acceptable levels of binding and/or other activities) were tested to determine if they showed reduced polyscores over the parental T18 molecule.

Heavy chain germlines:
human_IGHV3-30*18/3-30-5*01_DP-49+IGHJ2*01 (as in T18)
human_IGHV3-23*01/3-23D*01_DP-47+IGHJ2*01
human_IGHV3-23*01/3-23D*01_DP-47+IGJH4*01/4*02/4*03
human_IGHV3-7*01/3-7*02/3-7*03_DP-54+IGJH4*01/4*02/4*03 (as In 1602)
human_IGHV3-7*01/3-7*02/3-7*03_DP-54+IGHJ2*01
human IGHV3-9*01_IGHJ2*01

### Light Chain Germlines:

human_IGKV3-15*01/3D-15*01_DPK21+IGKJ3*01 (as In T18 & 1602)
human_IGKV3-11*01+IGKJ3*01
human_IGKV3-20*01_DPK22+IGKJ3*01

TABLE 16

| | | | | | Fold Diff |
|---|---|---|---|---|---|
| | | | | | EC50 (OAA |
| | | | | | format in IgA |
| | | AC- | | | Ramos by |
| IgG | GERMLINE | SINS | DNA | Insulin | FLOW |
| GBT-IgA-0027 | IGHV3-30*18 + IGHJ2*01 | 16 | 9 | 8 | 1 |
| | IGKV3-15*01 + IGKJ3*01 | | | | |
| GBT-IgA-0439 | IGHV3-7*01 + IGJH4*01, 02, 03 | 11 | 4 | 5 | 1.73 |
| | IGKV3-15*01 + IGKJ3*01 | | | | |
| GBT-IgA-0608 | IGHV3-9*01_IGHJ2*01 | 6 | 4 | 4 | 1.27 |
| | IGKV3-15*01 + IGKJ3*01 | | | | |

Germlines IGHV3-7*01_IGHJ4*01,02,03 with IGKV3-15*01_IGKJ3*01 and IGHV3-9*01_IGHJ2*01/IGKV3-15*01_IGKJ3*01 showed near equivalent binding on IgA Ramos cells and had reduced polyscores compared top parental T18. In addition, human_IGHV3-7*01+IGJH4*01/4*02/4*03 (as in 1602 molecule) showed improved polyscores over parental (T18 FW)

1. An isolated antibody or antigen-binding fragment thereof that specifically binds to human MIGIS-α, comprising CDRs selected from the one of the groups in the following list:
(i) a CDR-H1 sequence comprising the amino acid sequence of SEQ ID NO: 8; a CDR-H2 sequence comprising the amino acid sequence of SEQ ID NO: 10; a CDR-H3 sequence comprising the amino acid sequence of SEQ ID NO: 11; a CDR-L1 sequence comprising the amino acid sequence of SEQ ID NO: 1; a CDR-L2 sequence comprising the amino acid sequence of SEQ ID NO: 4; and a CDR-L3 sequence comprising the amino acid sequence of SEQ ID NO: 7;
(ii) a CDR-H1 sequence comprising the amino acid sequence of SEQ ID NO: 9; a CDR-H2 sequence comprising the amino acid sequence of SEQ ID NO: 10; a CDR-H3 sequence comprising the amino acid sequence of SEQ ID NO: 12; a CDR-L1 sequence comprising the amino acid sequence of SEQ ID NO: 1; a CDR-L2 sequence comprising the amino acid sequence of SEQ ID NO: 4; and a CDR-L3 sequence comprising the amino acid sequence of SEQ ID NO: 7;
(iii) a CDR-H1 sequence comprising the amino acid sequence of SEQ ID NO: 8; a CDR-H2 sequence comprising the amino acid sequence of SEQ ID NO: 10; a CDR-H3 sequence comprising the amino acid sequence of SEQ ID NO: 11; a CDR-L1 sequence comprising the amino acid sequence of SEQ ID NO: 2; a CDR-L2 sequence comprising the amino acid sequence of SEQ ID NO: 4; and a CDR-L3 sequence comprising the amino acid sequence of SEQ ID NO: 7;
(iv) a CDR-H1 sequence comprising the amino acid sequence of SEQ ID NO: 8; a CDR-H2 sequence comprising the amino acid sequence of SEQ ID NO: 10; a CDR-H3 sequence comprising the amino acid sequence of SEQ ID NO: 13; a CDR-L1 sequence comprising the amino acid sequence of SEQ ID NO: 3; a CDR-L2 sequence comprising the amino acid sequence of SEQ ID NO: 5; and a CDR-L3 sequence comprising the amino acid sequence of SEQ ID NO: 7;
(v) a CDR-H1 sequence comprising the amino acid sequence of SEQ ID NO: 8; a CDR-H2 sequence comprising the amino acid sequence of SEQ ID NO: 10; a CDR-H3 sequence comprising the amino acid sequence of SEQ ID NO: 11; a CDR-L1 sequence comprising the amino acid sequence of SEQ ID NO: 3; a CDR-L2 sequence comprising the amino acid sequence of SEQ ID NO: 5; and a CDR-L3 sequence comprising the amino acid sequence of SEQ ID NO: 7;

2. The antibody of claim 1, comprising a MIGIS-α VH framework sequence derived from a human germline selected from the group consisting of IGHV3-7*01, IGHV3-30*18, IGHV3-23*01, IGHV3-23*01, and IGHV3-9*01.

3. The antibody of claim 1, comprising a MIGIS-α VL framework sequence derived from a human germline selected from the group consisting of IGKV3-15*01, IGKV3-11*01, and IGKV3-20*01.

4. The antibody of claim 1, comprising a heavy chain variable region (VH) and a light chain variable region (VL) selected from the one of the groups in the following list:
(i) a VH comprising the amino acid sequence of SEQ ID NO: 14 and a VL comprising the amino acid sequence of 21;
(ii) a VH comprising the amino acid sequence of SEQ ID NO: 14 and a VL comprising the amino acid sequence of SEQ ID NO: 34;
(iii) a VH comprising the amino acid sequence of SEQ ID NO: 18 and a VL comprising the amino acid sequence of SEQ ID NO: 21; and
(iv) a VH comprising the amino acid sequence of SEQ ID NO: 15 and a VL comprising the amino acid sequence of SEQ ID NO: 21;
(v) a VH comprising the amino acid sequence of SEQ ID NO: 19 and a VL comprising the amino acid sequence of SEQ ID NO: 33; and
(vi) a VH comprising the amino acid sequence of SEQ ID NO: 19 and a VL comprising the amino acid sequence of SEQ ID NO: 21.

5. The antibody of claim 1, comprising a heavy chain comprising an amino acid according to SEQ ID NO: 48, and a light chain comprising an amino acid according to SEQ ID NO: 57.

6. The antibody of claim 1, comprising a heavy chain comprising an amino acid according to SEQ ID NO: 50, and a light chain comprising an amino acid according to SEQ ID NO: 51.

7. An isolated antibody that binds to human MIGIS-α, wherein the antibody binds to an epitope on human MIGIS-α located between residue M10 and residue L19, wherein the numbering is according to SEQ ID NO: 78.

8. The antibody of claim 1, wherein the antibody specifically binds to CD3 through a CD3 binding domain.

9. The antibody of claim 8, wherein the antibody specifically binds to CD3 through a CD3 binding domain, comprising a CD3-binding heavy chain variable region (CD3-VH) and a CD3-binding light chain variable region (CD3-VL), wherein the CD3-VH comprises a CDRH1 sequence of SEQ ID NO: 58; a CDRH2 sequence of SEQ ID NO: 59; and a CDRH3 sequence of SEQ ID NO: 60; and the CD3-VL comprises a CDRL1 sequence of SEQ ID NO: 61; a CDRL2 sequence of SEQ ID NO: 62, and a CDRL3 sequence of SEQ ID NO: 63.

10. The antibody of claim 9, wherein the CD3-VH comprises a CDRH1 sequence of SEQ ID NO: 58; a CDRH2 sequence of SEQ ID NO: 59; and a CDRH3 sequence of SEQ ID NO: 60; and the CD3-VL comprises a CDRL1 sequence of SEQ ID NO: 61; a CDRL2 sequence of SEQ ID NO: 62, and a CDRL3 sequence of SEQ ID NO: 63; and wherein the MIGIS-α-VH comprises a CDRH1 sequence of SEQ ID NO: 1; a CDRH2 sequence of SEQ ID NO: 4, and a CDRH3 sequence of SEQ ID NO: 7, and the MIGIS-α-VL comprises a CDRL1 sequence of SEQ ID NO: 8; a CDRL2 sequence of SEQ ID NO: 10, and a CDRL3 sequence of SEQ ID NO: 11.

11. The antibody of claim 10, wherein the MIGIS-α VH comprises an amino acid sequence of SEQ ID NO: 14, the MIGIS-α VL comprises an amino acid sequence of SEQ ID NO: 21, the CD3 VH comprises an amino acid sequence of SEQ ID NO: 66, and the CD3 VL comprises an amino acid sequence of SEQ ID NO: 67.

12. The antibody of claim 11, comprising a MIGIS-α HC comprising an amino acid sequence of SEQ ID NO: 48, a MIGIS-α LC comprising an amino acid sequence of SEQ ID NO: 57, a CD3 HC comprising an amino acid sequence of SEQ ID NO: 64, and a CD3 LC comprising an amino acid sequence of SEQ ID NO: 65.

13. The antibody of any one of claim 8, wherein the MIGIS-α binding domain comprises the HC sequence encoded by the plasmid deposited at the ATCC and having ATCC Accession No. PTA-127796, and the LC sequence encoded by the plasmid deposited at the ATCC and having ATCC Accession No. PTA-127794, and wherein the antibody further binds to CD3 through a CD3 binding domain, and the CD3 binding domain comprises the HC sequence encoded by the plasmid deposited at the ATCC and having ATCC Accession No. PTA-127797 and the LC sequence encoded by the plasmid deposited at the ATCC and having ATCC Accession No. PTA-127795.

14. The antibody of any one of claim 8, wherein the MIGIS-α binding domain comprises the HC sequence encoded by a nucleic acid sequence of SEQ ID NO: 17 and a MIGIS-α-LC sequence encoded by a nucleic acid sequence of SEQ ID NO: 16, and comprising a CD3 binding domain comprising a CD3-HC sequence encoded by a nucleic acid sequence of SEQ ID NO: 55, and a CD3-LC sequence encoded by a nucleic acid sequence of SEQ ID NO: 49.

15. The antibody of claim 8, wherein the CD3 binding domain comprises a VH and VL domain comprising CDRs identical to the CDRs of an anti CD3 antibody selected from the group consisting of teplizumab, glofitamab, mosunetuzmab, epcoritamab, otelixizumab, visilizumab, foralumab, and muromonab.

16. An isolated nucleic acid molecule, comprising one or both of
a. one or more nucleotide sequences encoding the VH or HC of the MIGIS-α binding domain of the antibody of claim 1, for use with one or more nucleic acid molecules encoding the VL or LC of the MIGIS-α binding domain of the antibody of claim 1; or
b. one or more nucleotide sequences encoding the VL or LC of the MIGIS-α binding domain of the antibody of claim 1, for use with a nucleic acid molecule encoding the VH or HC of the MIGIS-α binding domain of the antibody of claim 1.

17. A vector comprising one or more nucleic acid molecules as set forth in claim 16.

18. A host cell comprising the nucleic acid molecule of claim 16.

19. The host cell of claim 18, wherein said cell is a mammalian cell.

20. A method of making an antibody or antigen-binding fragment thereof, comprising culturing the host cell of claim 19, under a condition wherein said antibody or antigen-binding fragment is expressed by said host cell.

21. A pharmaceutical composition comprising an antibody of claim 1, and a pharmaceutically acceptable carrier or excipient.

22. A method of reducing the activity of IgA bound to B cells, comprising administering to a subject in need thereof a therapeutically effective amount of the antibody, or antigen-binding fragment thereof, of claim 1.

23. A method of treating an one or more conditions selected from the group consisting of IgA nephropathy (IgAN), Berger's disease, primary immunoglobulin A (IgA) nephropathy at risk of rapid disease progression, glomerular proteinuria, hematuria, inflammation of the glomeruli, glomerulonephritis, chronic glomerulonephritis, acute glomerulonephritis, Celiac Disease, and Henoch-Schonlein purpura, multiple myeloma (IgA subtype), IgA plasma cell neoplasms, Barrett's Esophagus, rheumatoid arthritis, COPD, Kawasaki Disease, IgA vasculitis, and idiopathic pulmonary fibrosis, comprising administering to a subject in need thereof a therapeutically effective amount of the antibody of claim 1.


SEQUENCE LISTING (90 sequences)

SEQ ID NO: 1 - protein, 10 aa, synthetic construct
GFEFSHYGMH

SEQ ID NO: 2 - protein, 10 aa, synthetic construct
GFTFSHYGMH

SEQ ID NO: 3 - protein, 10 aa, synthetic construct
GFTFSSYGMH

SEQ ID NO: 4 - protein, 17 aa, synthetic construct
VISYDASHKYYADSVKG

SEQ ID NO: 5 - protein, 17 aa, synthetic construct
VISYDGSNKYYADSVKG

SEQ ID NO: 6 - protein, 12 aa, synthetic construct
LYGGNPEDAIDY

SEQ ID NO: 7 - protein, 12 aa, synthetic construct
LYGGNPKDTIYH

SEQ ID NO: 8 - protein, 11 aa, synthetic construct
RASQSVSSNLA

SEQ ID NO: 9 - protein, 11 aa, synthetic construct
RASQSVSSDLA

SEQ ID NO: 10 - protein, 7 aa, synthetic construct
GASTRAT

SEQ ID NO: 11 - protein, 10 aa, synthetic construct
QQYNNWPPFT

SEQ ID NO: 12 - protein, 10 aa, synthetic construct
QTNHHWPPFT

SEQ ID NO: 13 - protein, 10 aa, synthetic construct
EQYKNWPPFT

SEQ ID NO: 14 - protein, 121 aa, synthetic construct
EVQLVESGGGLVQPGGSLRLSCAASGFEFSHYGMHWVRQAPGKGLEWVAVISYDASHKYYADSVKGRFTISRDNAKNSLYLQMNSLRAEDTAVYYCAKLYGGNPKDTIYHWGQGTLVTVSS

SEQ ID NO: 15 - protein, 121 aa, synthetic construct
EVQLVESGGGLVQPGGSLRLSCAASGFTFSHYGMHWVRQAPGKGLEWVAVISYDASHKYYADSVKGRFTISRDNAKNSLYLQMNSLRAEDTAVYYCAKLYGGNPKDTIYHWGQGTLVTVSS

SEQ ID NO: 16 - DNA, 645 bp, synthetic construct
  source 1..645: mol_type = other DNA
gaaattgtgttgacgcagtctccagccaccctgtctgtgtctccaggggaaagagccaccctctcctgcagggccagtcagagtgttagcagcaacttagcctggtaccagcaaaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctcctttcactttcggccctgggaccaaagtggatatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 17 - DNA, 1353 bp, synthetic construct
  source 1..1353: mol_type = other DNA
gaggtgcagctggtggagtctgggggaggcttggtccagcctggggggtccctgagactctcctgtgcagcctctggattcgagtttagtcattatggcatgcactgggtccgccaggctccagggaaggggctggagtgggtggccgttatatcatatgatgcaagtcacaaatactatgcagactctgtgaagggccgattcaccatctccagagacaacgccaagaactcactgtatctgcaaatgaacagcctgagagccgaggacacggctgtgtattactgtgcgaaactctacggtggtaatcctaaggatactatttaccactggggccagggcaccctggtcaccgtctcctcagcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtcggaaaactcacacatgcccaccgtgcccagcacctgaagccgctggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaggctcaccgtggacaagagcaggtggcagcaggggaacgtcttctcatgctccgtgatgcatgaggctctgcacaaccactacacgcagaagagcctctccctgtccccgggaaaa

SEQ ID NO: 18 - protein, 121 aa, synthetic construct
QVQLVESGGGVVQPGRSLRLSCAASGFTFSHYGMHWVRQAPGKGLEWVAVISYDASHKYYADSVKGRFTISRDNSKNTLYLQMNSLRAEDTAVYYCAKLYGGNPKDTIYHWGRGTLVTVSS

SEQ ID NO: 19 - protein, 121 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKLYGGNPKDTIYHWGRGTLVTVSS

SEQ ID NO: 20 - protein, 121 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKLYGGNPEDAIDYWGRGTLVTVSS

SEQ ID NO: 21 - protein, 108 aa, synthetic construct
EIVLTQSPATLSVSPGERATLSCRASQSVSSNLAWYQQKPGQAPRLLIYGASTRATGIPARFSGSGSGTEFTLTISSLQSEDFAVYYCQQYNNWPPFTFGPGTKVDIK

SEQ ID NO: 22 - protein, 10 aa, synthetic construct
GFTFSSYAMS

SEQ ID NO: 23 - protein, 10 aa, synthetic construct
GFTFSRYAMS

SEQ ID NO: 24 - protein, 17 aa, synthetic construct
SITGSGGGTYYADPVKG

SEQ ID NO: 25 - protein, 17 aa, synthetic construct
AITGSGSGTYYADPVKG

SEQ ID NO: 26 - protein, 9 aa, synthetic construct
ERSGSSSDY

SEQ ID NO: 27 - protein, 11 aa, synthetic construct
RASQTVSSYLA

SEQ ID NO: 28 - protein, 11 aa, synthetic construct
RASQTVRSHLA

SEQ ID NO: 29 - protein, 11 aa, synthetic construct
RASQTVGSHLA

SEQ ID NO: 30 - protein, 11 aa, synthetic construct
RASQTVSSHLA

SEQ ID NO: 31 - protein, 7 aa, synthetic construct
EASNRAT

SEQ ID NO: 32 - protein, 9 aa, synthetic construct
QQRNNWPIT

SEQ ID NO: 33 - protein, 108 aa, synthetic construct
EIVLTQSPATLSVSPGERATLSCRASQSVSSNLAWYQQKPGQAPRLLIYGASTRATGIPARFSGSGSGTEFTLTISSLQSEDFAVYYCEQYKNWPPFTFGPGTKVDIK

SEQ ID NO: 34 - protein, 108 aa, synthetic construct
EIVLTQSPATLSVSPGERATLSCRASQSVSSDLAWYQQKPGQAPRLLIYGASTRATGIPARFSGSGSGTEFTLTISSLQSEDFAVYYCQTNHHWPPFTFGPGTKVDIK

SEQ ID NO: 35 - protein, 118 aa, synthetic construct
EVQLLESGGGLVQPGGSLRLSCAASGFTFSSYAMSWVRQVPGKGLEWVSAITGSGSGTYYADPVKGRFTISRDNSKNTLYLQMNSLRAEDTAVYHCAKERSGSSSDYWGQGTLVTVSS

SEQ ID NO: 36 - protein, 107 aa, synthetic construct
EIVLTQSPATLSLSPGERATLSCRASQTVSSYLAWYQQKPGQAPRLLIYEASNRATGIPPRFSGSGSGTDFTLTISSLEPEDFAVYYCQQRNNWPITFGQGTRLESK

SEQ ID NO: 37 - protein, 118 aa, synthetic construct
EVQLLESGGGLVQPGGSLRLSCAASGFTFSSYAMSWVRQVPGKGLEWVSSITGSGGGTYYADPVKGRFTISRDNSKNTLYLQMNSLRAEDTAVYHCAKERSGSSSDYWGQGTLVTVSS

SEQ ID NO: 38 - protein, 107 aa, synthetic construct
EIVLTQSPATLSLSPGERATLSCRASQTVRSHLAWYQQKPGQAPRLLIYEASNRATGIPPRFSGSGSGTDFTLTISSLEPEDFAVYYCQQRNNWPITFGQGTRLESK

SEQ ID NO: 39 - protein, 118 aa, synthetic construct
EVQLLESGGGLVQPGGSLRLSCAASGFTFSRYAMSWVRQVPGKGLEWVSAITGSGSGTYYADPVKGRFTISRDNSKNTLYLQMNSLRAEDTAVYHCAKERSGSSSDYWGQGTLVTVSS

SEQ ID NO: 40 - protein, 107 aa, synthetic construct
EIVLTQSPATLSLSPGERATLSCRASQTVGSHLAWYQQKPGQAPRLLIYEASNRATGIPPRFSGSGSGTDFTLTISSLEPEDFAVYYCQQRNNWPITFGQGTRLESK

SEQ ID NO: 41 - protein, 118 aa, synthetic construct; identical to SEQ ID NO: 39

SEQ ID NO: 42 - protein, 107 aa, synthetic construct
EIVLTQSPATLSLSPGERATLSCRASQTVSSHLAWYQQKPGQAPRLLIYEASNRATGIPPRFSGSGSGTDFTLTISSLEPEDFAVYYCQQRNNWPITFGQGTRLESK

SEQ ID NO: 43 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 44 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 232 aa, synthetic construct
EPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 46 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - protein, 107 aa, Homo sapiens [sequence omitted]

SEQ ID NO: 48 - protein, 451 aa, synthetic construct
EVQLVESGGGLVQPGGSLRLSCAASGFEFSHYGMHWVRQAPGKGLEWVAVISYDASHKYYADSVKGRFTISRDNAKNSLYLQMNSLRAEDTAVYYCAKLYGGNPKDTIYHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 49 - DNA, 657 bp, synthetic construct
  source 1..657: mol_type = other DNA
gacatccagatgacacagagccctagcagcctgtctgccagcgtgggagacagagtgaccatcacctgtaccagcagccagagcctgttcaacgtgcggagccagaagaactacctggcctggtatcagcagaagcccggcaaggctcctaagctgctgatctactgggccagcaccagagaaagcggcgtgccaagcagattttctggcagcggctctggcaccgacttcaccctgaccatatctagcctgcagcctgaggacttcgccacctactactgcaagcagagctacgacctgttcaccttcggcggaggcaccaaggtggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 50 - protein, 451 aa, synthetic construct; identical to SEQ ID NO: 48

SEQ ID NO: 51 - protein, 215 aa, synthetic construct
EIVLTQSPATLSVSPGERATLSCRASQSVSSDLAWYQQKPGQAPRLLIYGASTRATGIPARFSGSGSGTEFTLTISSLQSEDFAVYYCQTNHHWPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 451 aa, synthetic construct
QVQLVESGGGVVQPGRSLRLSCAASGFTFSHYGMHWVRQAPGKGLEWVAVISYDASHKYYADSVKGRFTISRDNSKNTLYLQMNSLRAEDTAVYYCAKLYGGNPKDTIYHWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 53 - protein, 541 aa, synthetic construct
MGWSCIILFLVATATGAHSQVQLVQSGAEVKKPGASVKVSCKASGYSFTYYGISWVRQAPGQGLEWMGWISTYNGNTNYARMLQGRVTMTTDTSTRTAYMELRSLRSDDTAVYYCARENYYGSGSYRGGMDVWGQGTTVTVSSASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLADWQMPPPYVVLDLPQETLEEETPGANLWPTTITFLTLFLLSLFYSTALTVTSVRGPSGNREGPQY

SEQ ID NO: 54 - protein, 451 aa, synthetic construct
EVQLVESGGGLVQPGGSLRLSCAASGFTFSHYGMHWVRQAPGKGLEWVAVISYDASHKYYADSVKGRFTISRDNAKNSLYLQMNSLRAEDTAVYYCAKLYGGNPKDTIYHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 55 - DNA, 1353 bp, synthetic construct
  source 1..1353: mol_type = other DNA
gaggtgcagctggttgaatctggcggaggactggttcagcctggcggatctctgagactgtcttgtgccgccagcggcttcaccttcagcgactactacatgacctgggtccgacaggcccctggcaaaggacttgagtgggtcgccttcatccggaaccaggccagaggctacaccagcgaccacaatcctagcgtgaagggcagattcaccatcagccgggacaacgccaagaacagcctgtacctgcagatgaactccctgagagccgaggacaccgccgtgtactactgcgccagagacagacccagctactacgtgctggattactggggccagggcaccaccgtgacagtgtctagcgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgagaaaactcacacatgcccaccgtgcccagcacctgaagccgctggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaagagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcgaggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggaaaa

SEQ ID NO: 56 - protein, 450 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKLYGGNPKDTIYHWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 57 - protein, 215 aa, synthetic construct
EIVLTQSPATLSVSPGERATLSCRASQSVSSNLAWYQQKPGQAPRLLIYGASTRATGIPARFSGSGSGTEFTLTISSLQSEDFAVYYCQQYNNWPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 10 aa, synthetic construct
GFTFSDYYMT

SEQ ID NO: 59 - protein, 19 aa, synthetic construct
FIRNQARGYTSDHNPSVKG

SEQ ID NO: 60 - protein, 10 aa, synthetic construct
DRPSYYVLDY

SEQ ID NO: 61 - protein, 17 aa, synthetic construct
TSSQSLFNVRSQKNYLA

SEQ ID NO: 62 - protein, 7 aa, synthetic construct
WASTRES

SEQ ID NO: 63 - protein, 8 aa, synthetic construct
KQSYDLFT

SEQ ID NO: 64 - protein, 451 aa, synthetic construct
EVQLVESGGGLVQPGGSLRLSCAASGFTFSDYYMTWVRQAPGKGLEWVAFIRNQARGYTSDHNPSVKGRFTISRDNAKNSLYLQMNSLRAEDTAVYYCARDRPSYYVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 219 aa, synthetic construct
DIQMTQSPSSLSASVGDRVTITCTSSQSLFNVRSQKNYLAWYQQKPGKAPKLLIYWASTRESGVPSRFSGSGSGTDFTLTISSLQPEDFATYYCKQSYDLFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 121 aa, synthetic construct
EVQLVESGGGLVQPGGSLRLSCAASGFTFSDYYMTWVRQAPGKGLEWVAFIRNQARGYTSDHNPSVKGRFTISRDNAKNSLYLQMNSLRAEDTAVYYCARDRPSYYVLDYWGQGTTVTVSS

SEQ ID NO: 67 - protein, 112 aa, synthetic construct
DIQMTQSPSSLSASVGDRVTITCTSSQSLFNVRSQKNYLAWYQQKPGKAPKLLIYWASTRESGVPSRFSGSGSGTDFTLTISSLQPEDFATYYCKQSYDLFTFGGGTKVEIK

SEQ ID NO: 68 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 450 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKLYGGNPKDTIYHWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 441 aa, synthetic construct
EIVLTQSPATLSVSPGERATLSCRASQSVSSNLAWYQQKPGQAPRLLIYGASTRATGIPARFSGSGSGTEFTLTISSLQSEDFAVYYCQQYNNWPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 450 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKLYGGNPKDTIYHWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 72 - protein, 449 aa, synthetic construct
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 214 aa, synthetic construct
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 450 aa, synthetic construct; identical to SEQ ID NO: 71

SEQ ID NO: 75 - protein, 455 aa, synthetic construct
QVQLVQSGAEVKKPGSSVKVSCKASGYTFSSYAISWVRQAPGQGLEWMGGISPIFGTANYAQKFQGRVTITADESTSTAYMELSSLRSEDTAVYYCARDAGRSSDVGWYVGAIDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 216 aa, synthetic construct
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 77 - protein, 36 aa, Homo sapiens
DRLAGSCSVADWQMPPPYVVLDLPQETLEEETPGAN

SEQ ID NO: 78 - protein, 32 aa, Homo sapiens
GSCCVADWQMPPPYVVLDLPQETLEEETPGAN

SEQ ID NO: 79 - protein, 246 aa, Homo sapiens
HPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGSCSVADWQMPPPYVVLDLPQETLEEETPGANLWPT

SEQ ID NO: 80 - protein, 32 aa, Homo sapiens
GSSSVADWQMPPPYVVLDLPQETLEEETPGAN

SEQ ID NO: 81 - protein, 32 aa, synthetic construct
GSCSVADWQMPPPYVVLDLPQETLEEETPGAN

SEQ ID NO: 82 - protein, 8 aa, synthetic construct
QKTIDRLA

SEQ ID NO: 83 - protein, 15 aa, synthetic construct
GGGGSGGGGSGGGGS

SEQ ID NO: 84 - protein, 55 aa, Homo sapiens
QKTIDRLAGSSSVADWQMPPPYVVLDLPQETLEEETPGANGGGGSGGGGSGGGGS

SEQ ID NO: 85 - protein, 41 aa, Homo sapiens
DWQMPPPYVVLDLPQETLEEETPGANGGGGSGGGGSGGGGS

SEQ ID NO: 86 - protein, 117 aa, synthetic construct
EVQLVESGGGLVQPGGSLRLSCAASGFTFSSYGMHWVRQAPGKGLEWVAVISYQADGSKYYADSVKGRFTISRDNKNLYLQMNSLRAEDTAVYYCAKLGGNPKDTIWGQGTLVTVSS

SEQ ID NO: 87 - protein, 118 aa, synthetic construct
EVQLLESGGGLVQPGGSLRLSCAASGFTFSSYAMSWVRQVPGKGLEWVSAITCSGSCTYYADPVKGRFTISRDNSKNTLYLQMNSLRAEDTAVYHCAKERSGSSSDYWGQGTLVTVSS

SEQ ID NO: 88 - protein, 110 aa, synthetic construct
EIVLTQSPATLSVSPGERATLSCRASQSLFNVRSVSSLAWYQQKPGQAPRLLIYGASTRATGIPARFSGSGSGTFTLTISSLQEDFAVYYCQQYNNWPPFTFGPGTKVIK

SEQ ID NO: 89 - protein, 121 aa, synthetic construct
EVQLVESGGGLVQPGRSLRLSCAASGFTFSSYGMHWVRQAPGKGLEWVAVISYDGSNKYYADSVKGRFTISRDNAKNSLYLQMNSLRAEDTAVYYCAKLYGGNPKDTIYHWGRGTLVTVSS

SEQ ID NO: 90 - protein, 121 aa, synthetic construct
EVQLVESGGGLVQPGGSLRLSCAASGFTFSSYGMHWVRQAPGKGLEWVAVISYDGSNKYYADSVKGRFTISRDNAKNSLYLQMNSLRAEDTAVYYCAKLYGGNPKDTIYHWGQGTLVTVSS